(19) United States
(12) Patent Application Publication
(10) Pub. No.: US 2025/0009779 A1
(43) Pub. Date: Jan. 9, 2025

(54) METHODS FOR TREATING VIRAL DISEASES

(71) Applicant: NEW YORK SOCIETY FOR THE RELIEF OF THE RUPTURED AND CRIPPLED, MAINTAINING THE HOSPITAL FOR SPECIAL S, New York, NY (US)

(72) Inventors: Franck Barrat, Eastchester, NY (US); Lionel Ivashkiv, New York, NY (US)

(21) Appl. No.: 18/699,060

(22) PCT Filed: Oct. 12, 2022
(86) PCT No.: PCT/US2022/046404

§ 371 Date: Apr. 5, 2024

Related U.S. Application Data
(60) Provisional application No. 63/347,715, filed on Jun. 1, 2022.
(60) Provisional application No. 63/255,336, filed on Oct. 13, 2021.

(51) Int. Cl. A61K 31/7072; A61K 31/20; A61K 31/343; A61K 31/365; A61K 31/403; A61K 31/405; A61K 31/4706; A61K 31/501; A61K 31/52; A61K 31/573; A61K 31/675; A61K 31/7068; A61K 38/13; A61K 38/21; A61K 39/42; A61P 31/14; A61P 37/02

(52) U.S. Cl. CPC A61K 31/7072; A61K 31/20; A61K 31/343; A61K 31/365; A61K 31/403; A61K 31/405; A61K 31/4706; A61K 31/501; A61K 31/52; A61K 31/573; A61K 31/675; A61K 31/7068; A61K 38/13; A61K 38/215; A61K 39/42; A61P 31/14; A61P 37/02

(57) ABSTRACT

This disclosure features methods to treat viral diseases and/or hypercytokinemia using compounds that disrupt the tricarboxylic acid (TCA) cycle and/or compounds that activate the unfolded protein response (UPR) in immune cells such as plasmacytoid dendritic cells. In some embodiments, the immune cells are dendritic cells, macrophages, T cells, B cells, natural killer cells, and/or neutrophils.

### CROSS-REFERENCE TO RELATED APPLICATIONS

This application claims the benefit of the filing date of U.S. Provisional Application No. 63/255,336, filed Oct. 13, 2021, and U.S. Provisional Application No. 63/347,715, filed Jun. 1, 2022, the contents of which are incorporated by reference in their entirety herein.

### TECHNICAL FIELD

This disclosure relates to compositions and methods for the treatment of viral diseases, including COVID-19.

### BACKGROUND

The coronavirus disease 2019 (COVID-19) pandemic is caused by the severe acute respiratory syndrome coronavirus 2 (SARS-CoV-2) that has already infected hundreds of millions of people worldwide and is responsible for millions of deaths (Dong, E., et al. The Lancet. Infectious diseases 20, 533-534, doi:10.1016/S1473-3099(20)30120-1 (2020); Siddiqi, H. K. & Mehra, M. R. J Heart Lung Transplant 39, 405-407, (2020); Dolken, L., et al. Viruses 13, (2021)). A subset of patients develop a severe form of the disease which is associated with the presence of a large set of pro-inflammatory cytokines and an excessively exaggerated immune response, the so-called cytokine storm or hypercytokinemia, that are produced by macrophages in the lungs (Tay, M. Z., et al. Nat Rev Immunol 20, 363-374, (2020); Abassi, Z., et al. Front Immunol 11, 1312, (2020); Huang, C. et al. Lancet 395, 497-506, (2020); Melms, J. C. et al. Nature 595, 114-119, (2021); Rendeiro, A. F. et al. Nature 593, 564-569, (2021); Liao, M. et al. Nat Med, 26, 842-844 (2020); Hadjadj, J. et al. Impaired type I interferon activity and inflammatory responses in severe COVID-19 patients. Science 369, 718-724, (2020). How the anti-viral response to SARS-CoV-2 can evolve into a hyperactivation syndrome with the recruitment and subsequent activation of macrophages in the lungs is currently unclear. CS is also associated with selected viral infections, particularly influenza, SARS, and hantavirus, and often results in mortality and morbidity (Oldstone MB, Rosen H., Curr Top Microbiol Immunol. 2014; 378:129-47.)

Safe and effective drugs to control the cytokine storm are lacking, and clinically, the treatment of the cytokine storm has proved difficult (Yang, L., et al. Sig Transduct Target Ther 6, 255 (2021). Thus, there is a tremendous need for effective therapeutics to counter the cytokine storm associated with viral diseases.

### SUMMARY

This disclosure relates to methods for treating viral diseases associated with the cytokine storm in a human subject using a compound that that disrupts the tri-carboxylic acid (TCA) cycle and/or activates the Unfolded Protein response (UPR) in cells (e.g., dendritic cells and macrophages).

In the first aspect, the disclosure features a method of treating a condition selected from the group consisting of a viral disease or hypercytokinemia in a human subject in need thereof, the method comprising administering to the subject a therapeutically effective amount of a compound that disrupts the tri-carboxylic acid (TCA) cycle in immune cells or a compound that activates the Unfolded Protein response (UPR) in immune cells in the subject.

In some embodiments, the hypercytokinemia comprises an overproduction of immune cells and pro-inflammatory cytokines into the lungs of the subject.

In some embodiments, the compound that activates the UPR activates the IRE1α-XBP1 signaling branch of the UPR in immune cells.

In some embodiments, the compound that activates the UPR is tunicamycin, or thapsigargin. In some embodiments, the compound that activates the UPR is IXA4.

In some embodiments, the immune cells are dendritic cells, macrophages, T cells, B cells, natural killer cells, and/or neutrophils.

In some embodiments, the compound that disrupts the tri-carboxylic acid (TCA) cycle is
(a) a compound of Formula I

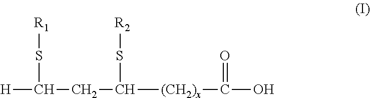

or a pharmaceutically acceptable salt thereof, wherein R₁ and R₂ are independently selected from the group consisting of acyl defined as R₃C(O)—, alkyl defined as CₙH₂ₙ₊₁, alkenyl defined as CₘH₂ₘ₋₁, alkynyl defined as CₘH₂ₘ₋₃, aryl, heteroaryl, alkyl sulfide defined as CH₃(CH₂)ₙ—S—, imidoyl defined as R₃C(═NH)—, hemiacetal defined as R₄CH(OH)—S—, and hydrogen provided that at least one of R₁ and R₂ is not hydrogen; wherein R₁ and R₂ as defined above can be unsubstituted or substituted; wherein R₃ is hydrogen, alkyl, alkenyl, alkynyl, cycloalkyl, aryl, alkylaryl, heteroaryl, or heterocyclyl, any of which can be substituted or unsubstituted; wherein R₄ is CCl₃ or COOH; and wherein x is 0-16, n is 0-10 and m is 2-10,
(b) UK5099 (PF-1005023)

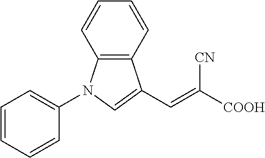 [SMILES: N#C/C(=C\c1cn(-c2ccccc2)c2ccccc12)C(=O)O]

or (c) CB839 (Telagenastat)

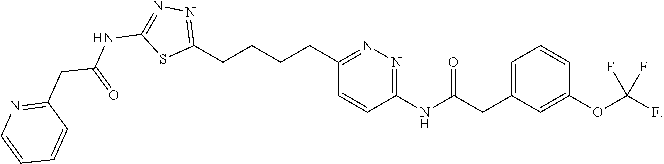 [SMILES: O=C(Cc1cccc(OC(F)(F)F)c1)Nc1ccc(CCCCc2nnc(NC(=O)Cc3ccccn3)s2)nn1]

In some embodiments, the R₁ and R₂ are benzyl or benzoyl.

In some embodiments, the compound of Formula I is

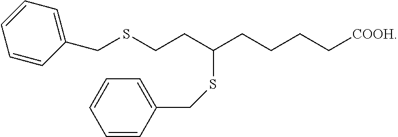 [SMILES: O=C(O)CCCCC(CCSCc1ccccc1)SCc1ccccc1]

In some embodiments, the compound of formula I is 6,8-bis-benzylthio-octanoic acid.

In some embodiments, the viral disease is coronavirus disease-19 (COVID-19), influenza, Severe Acute Respiratory Syndrome (SARS), or Hantavirus Pulmonary Syndrome (HPS).

In some embodiments, the subject is concurrently treated with one or more agents selected from the group consisting of a corticosteroid, remdesivir, Nirmatrelvir, Bebtelovimab, Molnupiravir, an IL-6 inhibitor, an IL-1 inhibitor, a kinase inhibitor, a complement inhibitor, ivermectin, hydroxychloroquine, favipiravir, interferon-beta and a nonsteroidal anti-inflammatory drug (NSAID). In some embodiments, the immunosuppressant is methotrexate, mycophenolate mofetil (MMF), cyclophosphamide, cyclosporin, or azathioprine. In some embodiments, the corticosteroid is hydrocortisone, methylprednisolone, dexamethasone or prednisone.

In some embodiments, the treatment reduces production of inflammatory cytokines or chemokines by dendritic cells in the human subject. In some embodiments, the inflammatory cytokines or chemokines are selected from the group consisting of: type I interferon (IFN-I), IL-6, or TNF-α, type III interferon, MIP-1a/CCL3, MIP-1/CCL4, CCL5/RANTES, and IP-10/CXCL10.

In some embodiments, the dendritic cells are plasmocytoid dendritic cells. In some embodiments, the dendritic cells express one or more of CD123, CD303 (BDCA2), CD304 (BDCA4), and immunoglobulin-like transcript 7 (ILT7). In some embodiments, the dendritic cells do not express the lineage-associated markers (Lin) CD3, CD19, CD14, CD16 and CD11c.

In some embodiments, the method inhibits and/or reduces IFN-I production in the human subject in need thereof by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, as compared to the corresponding reference levels in the human subject or in a control.

In some embodiments, the treatment reduces the expression of one or more of the interferon stimulated genes selected from the group consisting of Guanylate Binding Protein 1 (GBP1), Interferon Regulatory Factor 7 (IRF7), interferon stimulated gene 54 (ISG54), myxovirus resistance protein B (MxB), and 2′-5′-Oligoadenylate Synthetase 2 (OAS2).

In some embodiments, the treatment enhances expression of phosphoglycerate dehydrogenase (PHGDH), phosphoserine Phosphatase (PSPH), and phosphoserine Aminotransferase 1 (PSAT1).

In another aspect, the disclosure features the use of a therapeutically effective amount of a compound that disrupts the tri-carboxylic acid (TCA) cycle or a compound activates the Unfolded Protein response (UPR) in immune cells in the subject to treat a condition selected from the group consisting of a viral disease and hypercytokinemia in the subject.

In some embodiments, the compound that disrupts the tri-carboxylic acid (TCA) cycle is (a) a compound of Formula I

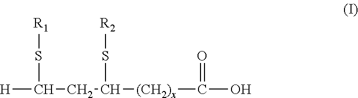

or a pharmaceutically acceptable salt thereof, wherein R₁ and R₂ are independently selected from the group consisting of acyl defined as R₃C(O)—, alkyl defined as CₙH₂ₙ₊₁, alkenyl defined as CₘH₂ₘ₋₁, alkynyl defined as CₘH₂ₘ₋₃, aryl, heteroaryl, alkyl sulfide defined as CH₃(CH₂)ₙ—S—, imidoyl defined as R₃C(═NH)—, hemiacetal defined as R₄CH(OH)—S—, and hydrogen provided that at least one of R₁ and R₂ is not hydrogen; wherein R₁ and R₂ as defined above can be unsubstituted or substituted; wherein R₃ is hydrogen, alkyl, alkenyl, alkynyl, cycloalkyl, aryl, alkylaryl, heteroaryl, or heterocyclyl, any of which can be substituted or unsubstituted; wherein R₄ is CCl₃ or COOH; and wherein x is 0-16, n is 0-10 and m is 2-10,
(b) UK5099 (PF-1005023)

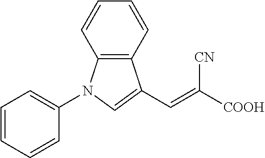 [SMILES: N#C/C(=C\c1cn(-c2ccccc2)c2ccccc12)C(=O)O]

or (c) CB839 (Telagenastat)

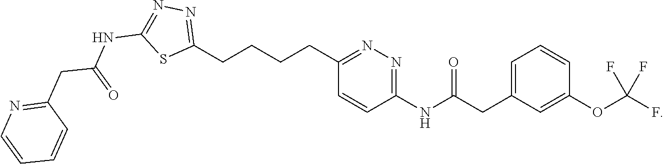 [SMILES: O=C(Cc1cccc(OC(F)(F)F)c1)Nc1ccc(CCCCc2nnc(NC(=O)Cc3ccccn3)s2)nn1]

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIGS. 1A-1N show the dynamics of IFN-α and inflammatory response in macrophages from bronchoalveolar lavage fluid and lung. FIGS. 1A-1B: UMAP representation of a scRNA-seq dataset from bronchoalveolar lavage of COVID-19 patients (n=3 mild and n=6 severe) and non-COVID-19 controls (n=4) (Liao, M. et al. Nature Medicine 26, pages842-844 (2020) showing the inferred cell type identities (FIG. 1A) and the disease state of the donor (FIG. 1B). (FIG. 1C) Abundance of macrophages depending on disease state. (FIG. 1D) UMAP plot as in FIG. 1A) but with each cell colored by the intensity of signatures related with IFN-I response, COVID-19 inflammation, and fibrosis. (FIG. 1E) Abundance of signatures from (c) for macrophages aggregated by disease state. (FIG. 1F) Correlation between the signatures of IFN-I response and COVID-19 inflammation across macrophage cells dependent on disease state. The dashed lines mark the mean value of the signatures across all cells. (FIG. 1G) Inference of a pseudotime axis using Diffusion Maps for macrophage cells only. The first axis (x-axis) reveals a dynamic transition between macrophages of healthy donors, mild COVID-19 patients and severe patients. (FIG. 1H) Distribution of macrophage cells (top), IFN-I response signature (middle), and COVID-19 inflammatory signature (bottom) across the pseudotime axis (panel g). (FIG. 1I) Abundance of pDCs depending on disease state. (FIG. 1J) Abundance of IFN-I response for pDCs aggregated by disease state. (FIG. 1K) Proportion of dendritic cells from each cluster that belong to COVID-19 patients or controls in lung tissue. Statistical significance was evaluated with a Fisher's exact test and p-values adjusted for multiple testing with Benjamini-Hochberg false discovery rate method (BH FDR). (FIG. 1L) Heatmap of mean inferred signature activity for pDCs and macrophages, dependent on disease status. Values were min-max scaled per signature to enable comparison. For the signatures, only windows with at least 100 cells are displayed. (FIGS. 1M-1N) IFN-I response, COVID-19 inflammatory genes and fibrotic genes in both alveolar (FIG. 1M) and monocyte-derived macrophages (FIG. 1N) from control and COVID-19 patient lungs. Statistical significance was evaluated with a Mann-Whitney U-test and p-values adjusted with BH-FDR method.

FIGS. 2A-2G shows SARS-CoV-2 sensing by TLR7 induces type I interferon by pDCs. (FIG. 2A) Purified pDCs from HDs (n=6 and 29) were cultured for 24 hours with or without either live SARS-CoV-2 or inactivated SARS-CoV-2 at MOI 0.25. Production of IFN-α was quantified by ELISA. (FIGS. 2B-2C) Purified pDCs from HDs (n=6) were cultured for 24 hours with live at MOI 0.25, 0.1 or 0.01. Gene expression of SARS-CoV-2 protein E (FIG. 2B) and N (FIG. 2C) were quantified by qPCR. (FIG. 2D) Production of IFN-α were quantified in total PBMC or pDC-depleted PBMC from HD (n=6) infected or not by live SARS-CoV-2 at MOI 0.25. Production of IFN-α was quantified by ELISA. (FIGS. 2E-2F) Gene expression of SARS-CoV-2 protein E (FIG. 2E) and N (FIG. 2F) were quantified by qPCR in total PBMC or pDC depleted PBMC from HD (n=6) infected or not by live SARS-CoV-2 at MOI 0.25. (FIG. 2G) Production of IFN-α were quantified in total PBMC or pDC-depleted PBMC from HD (n=6) infected or not by inactivated Flu at MOI 2. Production of IFN-α was quantified by ELISA. All results are represented as means±SEM. Statistical significance was evaluated using a Friedman test with Dunn's multiple comparisons posttest or a Mann-Whitney test *P<0.05, **P<0.01, ***P<0.001. ns=not significant.

FIGS. 3A-3H show that SARS-activated pDCs exacerbate TLR4 signaling in macrophages via IFN pathway. (FIG. 3A) Supernatant of unstimulated or inactivated SARS-CoV-2 stimulated pDCs were pre-incubated with macrophages for 24 hours following by the addition of TLR agonists for 6 hours. (FIG. 3B) Macrophages from HDs (n=20) were pre-incubated for 24 hours with supernatants (SN) of either non-activated (Unst-pDC SN) or SARS-pDCs (SARS-pDC SN) following by the addition, or not, of LPS (10 ng/ml) for 6 hours. Expression levels of TNF and IL-6 were quantified by qPCR. (FIGS. 3C-3E) Macrophages from HDs (n=5-8) were pre-incubated for 24 hours with supernatants (SN) of either non-activated (Unst-pDC SN) or SARS-pDCs (SARS-pDC SN) following by the addition, or not, of (FIG. 3B) Pam3Cys (20 ng/ml), (FIG. 3C) PolyIC (10 μg/ml) or (FIG. 3D) ORN8L (60 μM) for 6 hours. Expression level of IL-6 was quantified by qPCR. (FIGS. 3F-3G) Macrophages from HDs (n=5-10) were pre-incubated for 24 hours with supernatant of SARS-pDCs in the presence of baricitinib (2 μM) and/or anti-IFNAR antibody (2 μg/ml) followed by the addition of LPS for 6 hours. Expression levels of TNF and IL-6 quantified by qPCR. (FIG. 3H) Macrophages from HDs (n=4) were pre-incubated either alone or with different concentrations of IFN-α as indicated (pg/ml) or with SARS-pDC SN and stimulated with LPS, or not. Expression levels of TNF and IL-6 quantified by qPCR. All results are represented as means±SEM. Statistical significance was evaluated using a Friedman test with Dunn's multiple comparisons posttest or a Mann-Whitney test *P<0.05, **P<0.01, ***P<0.001. ns=not significant.

FIGS. 4A-4F shows that IFN-α increase inflammatory transcription and chromatin accessibility in macrophages. (FIG. 4A) Principal component analysis (PCA) of the differentiated genes in either unstimulated (unst), IFN-α-incubated or SARS-pDC SN-incubated macrophages following, when indicated, by the addition of LPS for 3 hours (2 ng/ml). PCI and 2 capture percent variation associated with either individual or combination treatments. (FIG. 4B) K-means clustering (K=7) of differentially expressed genes induced by more than a two-fold change with FDR<0.05 in the conditions shown in a. (FIG. 4C) Macrophages (n=4) were incubated for 24 hours with either Unst-pDC SN, IFN-α or SARS-pDC SN following, or not, by the addition of LPS. Number of genes differentiated by IFN-α, SARS-pDC SN or LPS were normalized to unst condition. (FIG. 4D) The top activated pathways of the differentiated genes i 700 nduced by more than two-fold with FDR<0.05 in macrophages pre-incubated with SARS-pDC SN and then cultured for 3 h with LPS versus LPS alone. (FIG. 4E) Heat-map showing the inflammatory genes related to COVID-19 in macrophages incubated under the same conditions as a. (FIG. 4F) Macrophages were cultured for 24 h with SARS-pDC SN either alone or with the TLR7 inhibitor IRS661 followed by the addition of LPS for 6 h. Gene expression levels and production of TNF and TL-6 were quantified by qPCR and ELISA respectively. (FIG. 4G) Macrophages were incubated alone (unst) or with IFN-α for 24 hours. LPS was then added (when indicated) for 3 hours and the FAIRE assay performed on the promoter regions of IL6, TNF and IL12p40. Results are represented as means±SEM (f, g). Statistical significance was evaluated using a Mann-Whitney test (FIG. 4F) and One-way ANOVA. *P<0.05, **P<0.01, ***P<0.001.

FIGS. 5A-5C show that the scRNA-seq of bronchoalveolar lavage of COVID-19 patients highlights dynamic cell composition and response (FIG. 5A) Expression of cell type defining markers in the UMAP representation of scRNA-seq data from bronchoalveolar lavage of COVID-19 patients and non-COVID-19 controls {Liao, 2020 #30028}. (FIG. 5B) Abundance of cell types depending on disease state. (c) Abundance of gene expression signatures aggregated by cell type and disease state.

FIGS. 6A-6C show that IFN-I is the main driver of disease progression between with an orthogonal approach. (FIG. 6A) Heatmap of genes differentially expressed in macrophages between the two groups of COVID-19 patients and between each group and Controls. The top 15 genes were selected per comparison for display. (FIG. 6B) Venn diagram of all differentially expressed genes between disease states. (FIG. 6C) Enrichment of the top 100 differentially expressed genes per disease group comparison in gene ontology terms. The top 10 terms for each comparison and FDR-adjusted pvalues were displayed.

FIGS. 7A-7B show that enrichment for the IFN-α and IFN-γ signaling pathways across cell types have distinct profiles. (FIG. 7A-7B) Mean enrichment (FIG. 7A) and correlation (FIG. 7B) of all MSigDB pathways in each cell types. Data were analyzed in an unsupervised manner. Enrichment for the IFN-α and IFN-γ signaling pathways across cell types have distinct profiles.

FIGS. 8A-811 show gene expression and cell cluster in lungs of COVID-19 patients. (FIG. 8A) UMAP representation of DCs from a scRNA sequencing dataset of lung tissue from COVID-19 patients4. Cells are colored by cluster identity, where cluster 5 are pDCs. (FIG. 8B) Pathways ranked by change in inferred activity between COVID-19 and controls in pDCs. (FIG. 8C) UMAP of scRNAseq dataset with cells colored by their cell type identity. The area inside the dashed line square is examined in panel B. (FIG. 8D) Enlargement of the area highlighted in A (where DCs in yellow). (FIG. 8E) Expression of markers specific to clusters of DCs in a new UMAP projection made from DCs only. (FIG. 8F) Mean expression of known markers of cell identity or function of DCs across the five clusters of DCs. (FIG. 8G) Mean expression of marker genes specific to each DC cluster discovered in an unsupervised manner. The top 10 genes of each cluster are displayed, and their values are Zscore transformed across rows. (FIG. 811) UMAP representation of DCs where cells are colored by the disease state of the donor (Control or COVID-19) (FIG. 8I) Heatmap of Hallmark pathway signatures across the major cell types found in lung tissue of Control or COVID-19 patients.

FIGS. 9A-9D show that IFN-α-induction in lungs of COVID-19 patients is associated with a cytokine storm by macrophages. (FIG. 9A) UMAP representation of all cells in snRNA-seq dataset colored by the inferred activity of signatures related to IFN-I response, COVID-19 inflammation, or Fibrosis. (FIG. 9B) Heatmap of mean inferred signature activity for all cell types, dependent on disease status. Values were min-max scaled per signature to enable comparison. (FIG. 9C) Correlation between signature values across single-cells of the same cell type in COVID-19. Significance of Pearson correlation is indicated by asterisks (**) when p<0.01, with p-values adjusted with Benjamini-Hochberg FDR method. (FIG. 9D) Scatter plot of the inferred activity of IFN-α and inflammatory signatures in macrophages from COVID-19 patients. Statistics indicate the effect size and significance of Pearson correlation. The line indicates the trend and 95th confidence interval of the data. Note the logarithmic scale of both axes.

FIGS. 10A-10N show that TLR7 sensing of SARS-CoV-2 by pDCs is necessary for the optimal induction of IFN-α by PBMC, while Flu can use other pathways. FIG. 10A Purified pDCs from HDs (n=6 and 8) were cultured for 24 hours with or without either live SARS-CoV-2 or inactivated SARS-CoV-2 at MOI 0.25. Gene expression of IFN-α was quantified by qPCR. (FIGS. 10B-10E) Gene expression and production levels of IFN-α were quantified in total PBMC or pDC-depleted PBMC from healthy donors (HD) infected or not by either (FIG. 10B) live SARS-CoV-2 at MOI 0.25 (n=6), (FIGS. 10C-10D) inactivated SARS-CoV-2 at MOI 0.25 (n=13) or (FIG. 10E) inactivated Flu (n=6) at MOI 2. Gene expression and production of IFN-α were quantified by qPCR and ELISA respectively. (FIG. 10F-10H) Purified pDCs from HDs (n=6 and 18) were cultured for 24 hours with or without either live SARS-CoV-2 or inactivated SARS-CoV-2 at MOI 0.25 in the presence or not of a TLR7 inhibitor IRS661 (2 μM), an ACE2 inhibitor (2 μM) or a PI3Kδ inhibitor CAL-101 (10 μM). Production and gene expression of IFN-α were quantified respectively by ELISA and qPCR. (FIGS. 10I-10J) PBMC were incubated either with live SARS-CoV2 (MOI=0.25), inactivated SARS-COV2 (MOI=0.25) or Flu (MOI=2) in the presence or not of a TLR7 inhibitor IRS661 (2 μM). Production and gene expression of IFN-α were quantified by ELISA and qPCR respectively. (FIG. 10K) pDC-depleted PBMC were incubated with Flu (MOI=2) in the presence or not of a TLR7 inhibitor IRS661 (2 μM). IFN-α production was quantified by ELISA. (FIGS. 10L-10N) Purified pDCs from HDs (n=8 to 18) were cultured for 24 hours with inactivated SARS-CoV-2 either alone or in the presence of chlathrin inhibitor chlorpromazine (CPZ, 30 μM) or dynamin inhibitor dynasore hydrate (DH, 100 μM). Production of IFN-α were quantified by ELISA, gene expression of IFN-α2 and SARS-CoV-2 ribonucleoprotein were quantified by qPCR. All results are represented as means SEM. Statistical significance was evaluated using a Friedman test with Dunn's multiple comparisons posttest or a Mann-Whitney test *P<0.05, **P<0.01, ***P<0.001. ns=not significant.

FIGS. 11A-11F show that SARS-CoV-2 induces type I interferons by pDCs. (FIG. 11A) Purified pDCs from HDs (n=3) were cultured for 24 hours with or without inactivated SARSCoV-2 at MOI 1, 0.5, 0.25 and 0.1. IFN-α production was quantified by ELISA. (FIG. 11B) IFN-α2 expression levels were quantified in unstimulated pDCs (Unst) and in pDCs activated with either CpG C274 (0.5 μM) or inactivated SARS-CoV-2 (MOI 0.25) for 3, 6, 10 and 18 hours (n=4). (FIG. 11C) Gene expression of SARS-CoV-2 ribonucleoprotein in pDCs (n=4) at 3 h, 6 h, 10 h and 18 h postinfection with SARS-CoV-2. (FIG. 11D-11F) Purified pDCs from HDs (n=4) were incubated for 6 h and 18 h either alone, with SARS-CoV-2 (MOI 0.25) or CpG C274 (0.5 μM). Expression levels of type I and type III IFNs were quantified by qPCR (rel Ct). Heatmap was generated with mean of each gene. (FIG. 11H-11I) Purified pDCs from HDs (n=4) were incubated for 6 h (FIG. 11H) and 18 h (i) either alone or with SARS-CoV-2 (MOI 0.25). Expression levels of CCL2, CCL5, CCL8 and CXCL8 were quantified by qPCR (rel Ct). All results are represented as means±SEM. Statistical significance was evaluated using a Friedman test with Dunn's multiple comparisons posttest (FIG. 11D-11F) or Mann-Whitney test (FIG. 11G). *P<0.05, **P<0.01, ***P<0.001. ns=not significant

FIGS. 12A-12F show that SARS-CoV-2 minimally activate macrophage response. (FIG. 12A) Pluripotent stem cells (PSCs) derived macrophages (PSC-Macs), monocyte derived macrophages (MoD-Macs), VERO cells and A549-ACE2 cells were infected for 48 h with live SARS-CoV-2 at 0.1 MOI. (FIG. 12B) Alveolar macrophages isolated from primary human lung tissue (Alveo-Macs) were polarized or not in either M1 or M2 conditions (using LPS/IFN-γ or IL-4/IL-10, respectively) and infected for 48 h with live SARS-CoV-2 at 0.1 MOI. (FIG. 12C-12D) Macrophages from HDs (n=8 and 5) were infected by inactivated SARS-CoV-2 for 24 hours. Production and gene expression of TNF and IL-6 were quantified by ELISA and qPCR respectively. (FIG. 12E-12F) Macrophages from HDs (n=5) were pre-incubated for 24 hours with supernatants (SN) of either non-activated (Unst-pDC SN) or SARS-pDCs (SARS-pDC SN) following by the addition, or not, of inactivated SARS-COV-2 for 6 hours. Expression levels and production of TNF and IL-6 were quantified by qPCR and ELISA respectively. All results are represented as means±SEM.

FIGS. 13A-13H show that SARS-pDCs exacerbate macrophage activation. (FIG. 13A) Macrophages from HDs (n=22) were pre-incubated for 24 hours with supernatants (SN) of either non-activated (Unst-pDC SN) or SARS-pDCs (SARS-pDC SN) following by the addition, or not, of LPS for 6 hours. Gene expression of CXCL10 was quantified by q-PCR. (FIGS. 13B-13D) Macrophages from HDs (n=5) were pre-incubated for 24 hours with supernatants (SN) of either non-activated (Unst-pDC SN) or SARS-pDCs (SARS-pDC SN) following by the addition, or not, of either Pam3Cys, PolyIC or ORN8L for 6 hours. TNF gene expression was quantified by q-PCR. (FIGS. 13E-13H) Macrophages from HDs (n=5 or 20) were pre-incubated for 24 hours with supernatants (SN) of either non-activated (Unst-pDC SN) or SARS-pDCs (SARS-pDC SN) following by the addition, or not, of either LPS, Pam3Cys, PolyIC or ORN8L for 6 hours. Production of TNF and IL-6 were quantified by ELISA. All results are represented as means±SEM. Statistical significance was evaluated using a Mann-Whitney test. *P<0.05, **P<0.01, ***P<0.001. ns=not significant.

FIG. 14 shows that IFN-α induces inflammatory genes in dose-dependent manner. Gene expression levels of IL-1β, IL-12p40, IFN-β and CXCL10 were quantified in unstimulated macrophages (Unst.) and in macrophages incubated with either IFN-α (100000, 30000, 10000, 3000, 1000 and 300 μg/ml) or supernatants of SARS-pDCs (SARS-pDC SN) for 24 hours. When indicated, LPS was added for 3 hours (2 ng/ml). All results are represented as means±SEM from HDs (n=4).

FIG. 15 shows that TNF produced by SARS-pDCs doesn't contribute to exacerbation of the macrophage response to LPS. Macrophages from HDs (n=4) were pre-incubated for 24 hours with supernatants (SN) of either non-activated (Unst-pDC SN) or SARS-pDCs (SARS-pDC SN) in the presence of anti-TNFR antibody (10 μg/ml) and anti-TNF antibody (10 μg/ml) followed by the addition of LPS for 6 hours. Expression levels of TNF and IL-6 quantified by qPCR. All results are represented as means±SEM.

FIGS. 16A-16B shows that the activation of macrophage by supernatants of SARS-pDCs is blocked by baricitinib (FIGS. 16A-16B) Macrophages from HDs (n=7) were incubated for 24 hours with supernatants of SARSpDCs alone or in the presence of baricitinib (2 μM). (FIG. 16A) Gene expression of CXCL10 was quantified by qPCR and (FIG. 16B) production of TNF and IL-6 were quantified by ELISA. All results are represented as means±SEM. Statistical significance was evaluated using a Mann-Whitney test. *P<0.05, **P<0.01, ***P<0.001.

FIGS. 17A-17H shows that the superinduction of macrophage activation by supernatants of SARS-pDCs is blocked by baricitinib and requires IFN-I signaling. (FIGS. 17A-17G) Macrophages from HDs (n=5-9) were pre-incubated for 24 hours with supernatants of SARS-pDCs in the presence of baricitinib (2 μM) followed by the addition of LPS, Pam3Cys, PolyIC and ORN8L for 6 hours. Production and gene expression of TNF and IL6 were quantified by ELISA and qPCR respectively. (FIG. 17H) Macrophages from HDs (n=3) were pre-incubated for 24 hours with supernatant of SARS-pDCs in the presence of baricitinib (2 μM) and/or anti-IFNAR antibody (2 μg/ml) followed by the addition of LPS for 6 hours. Production of TNF and IL6 were quantified by ELISA. All results are represented as means±SEM. Statistical significance was evaluated using a Mann-Whitney test (a-g) or Friedman test with Dunn's multiple comparisons posttest (FIG. 1711). *P<0.05, **P<0.01, ***P<0.001.

FIGS. 18A-18E show that IFN-α increases TLR4-induced gene expression but not TLR4-mediated signaling pathway activation. (FIG. 18A) The top pathways analysis by IPA of the differentiated genes in each cluster from FIG. 4B. (FIG. 18B-18C) The top GO_BP (FIG. 18B) and TF (FIG. 18C) enrichment of the differentiated expressed genes in the indicated conditions by InnateDB (Cardani, A., et al. PLoS Pathog 13, e1006140 (2017). (FIGS. 18A-18D) Heat-map showing the fibrosis pathway associated gene expression in the indicated conditions. (FIG. 18E) Heat-map showing ISGs in the indicated conditions. (FIG. 18F) Heat-map showing chemokine receptors in the indicated conditions. Gene and protein expressions of IL6 and TNF in macrophage incubated with SARS-pDC SN cultured with the PI3Kδ inhibitor or not, and then with LPS.

FIG. 19 shows the effect of LPS, IFN-α or supernatants of SARS-pDCs on IFNAR1, IFNAR2 and TLR4 expression levels. Macrophages (n=4) were incubated for 24 hours with either Unst-pDC SN, IFN-α or SARS-pDC SN following, or not, by the addition of LPS. Expression of IFNAR1, IFNAR2 and TLR4 were quantified by Log 2 CMPs (count per million).

FIG. 20 shows that the pDCs require TLR7 signaling and PI3Kδ in response to SARSCoV-2 in order to superinduce macrophage response to stimuli, while IFN-α does not increase TLR4-mediated signaling pathway activation. (FIG. 5A a) Macrophages were cultured for 24 h with SARS-pDC SN either alone or with the PI3Kδ inhibitor CAL-101 (10 μM) followed by the addition of LPS for 6 h. Gene expression levels and production of TNF and IL-6 were quantified by qPCR and ELISA respectively. (FIG. 5A b) Immunoblot of NF-κB and MAPKs signaling with whole cell lysates of the indicated conditions. All results are represented as means±SEM. Statistical significance was evaluated using a Mann-Whitney test. *P<0.05, **P<0.01, ***P<0.001

FIG. 21 is a schematic showing that the sensing of SARS-CoV-2 and IFN-I production by pDCs promotes the cytokine storm by macrophages.

FIG. 22A shows the gene enrichment score of amino acid biosynthesis. FIG. 22B shows heatmaps of genes involved in in amino acid biosynthesis. Arrows indicate genes that have low expression (0 to −6 represents expression of less than 1 count per million with −6 representing log 2 (−6) count per million); no arrows indicate genes with expression of more than 1 count per million with 8 representing log 2 (8) count per million. FIG. 22C shows Volcano plot comparing gene expression of serine biosynthesis analyzed by RNA-seq of pDCs from HDs cultured for 8 h with TLR9 agonist and tunicamycin vs TLR9 agonist alone. For FIGS. 22A-22C, purified pDCs from HDs were cultured in medium or with tunicamycin (TM) for 3 h, followed by TLR9 agonist for 5 h.

FIG. 22D shows gene expression of PHGDH when pDCs were cultured in media alone or with tunicamycin or thapsigargin for 3 h, followed by TLR9 agonist for 5 h. FIG. 22E shows gene expression level of PHGDH quantified and normalized to tunicamycin treatment when pDCs were cultured with tunicamycin alone or in combination with tunicamycin and IRE1a inhibitor (MKC8866 at 1 μM) for 8 h. FIG. 22F shows gene expression level of PHGDH quantified and normalized to medium when pDCs were cultured in medium or with IRE1α-XBP1 agonist (IXA4 at 30 μM) for 6 h. FIG. 22G shows the graphical representation of the role of PHGDH in glucose metabolism. FIGS. 22H and 22I show gene expression level of IFNA quantified at 5 h and normalized to TLR9 agonist treatment. pDCs were cultured in medium or with tunicamycin (TM) or thapsigargin (TG) in combination with PHGDH inhibitor (NCT-503 at 2M) for 3 h, when TLR9 agonist was added to culture. FIG. 22J shows secreted IFN-α was quantified by ELISA after 13 h of culture. pDCs were cultured in medium or with L-serine (1 mg/ml) for 1 h when TLR9 agonist was added to culture. FIGS. 22K and 22L show intracellular pyruvate when pDCs were cultured with tunicamycin (TM) and thapsigargin (TG) in combination with PHGDH inhibitor (NCT-503 at 2 μM) for 3 h, and TLR9 agonist was added to the culture for 2.5 h. Individual donors are indicated, and all results are represented as a mean±SEM and statistical significance was evaluated using a Mann-Whitney U-test. *p<0.05, **p<0.01, ***p<0.001.

FIGS. 23A and 23B show gene expression level of IFNA quantified at 5 h and normalized to TLR9 agonist treatment. FIGS. 23C and 23D show gene expression level of IFNA quantified at 5 h and normalized to TLR9 agonist treatment. FIGS. 23E and 23F show intracellular ATP quantified using ATP assay kit after 2.5 h of culture and normalized to medium. FIGS. 23G and 23H show intracellular ATP quantified after 2.5 h of culture and normalized to medium. FIG. 23I shows intracellular ATP quantified after 2.5 h of culture and normalized to medium. pDCs were cultured in medium or with tunicamycin in combination with PHGDH inhibitor (NCT-503 at 2M) for 3 h, when TLR9 agonist was added to culture. FIG. 23J shows gene expression level of IFNA quantified at 5 h and normalized to TLR9 agonist treatment. FIG. 23K shows secreted IFN-α quantified by ELISA after 13 h of culture. FIG. 23L shows intracellular ATP quantified after 4 h of culture and normalized to medium. For FIGS. 23A, 23B, 23E, and 23F, pDCs were cultured with tunicamycin or thapsigargin alone or with sodium pyruvate (pyruvate at 10 mM) for 3 h, when TLR9 agonist was added to the culture. For FIGS. 23C, 23D, 23G, and 23H, pDCs were cultured with tunicamycin or thapsigargin alone or with α-ketoglutaric acid disodium salt (α-KG at 10 mM) for 3 h, when TLR9 agonist were added to the culture. For FIGS. 23J-23L, pDCs were cultured in medium or with inhibitor for PDH and α-KGDH (CPI-613 at 100 μM & 200 μM) for 1 h, when TLR9 agonist was added to the culture. Individual donors are indicated, and all results are represented as a mean±SEM and statistical significance was evaluated using a Mann-Whitney U test. *p<0.05, **p<0.01, ***p<0.001.

FIGS. 24A-24E shows gene expression level of the ISGs GBP1, IRF7, CXCL10, MxB, ISG54. FIG. 24F shows cell viability quantified by flow cytometer. FIGS. 24G and 24H show gene expression level of spliced XiBP1 and of PHGDH quantified by Q-PCR. For FIGS. 24A-24H, human pDCs were cultured in medium or with an inhibitor for both PDH and α-KGDH (CPI-613 at 100 & 200 μM) for 1 h, when TLR9 agonist was added to the culture for 5 h. Individual donors are indicated, and all results are represented as a mean±SEM and statistical significance was evaluated using a Mann-Whitney U test. ns p>0.05, *p<0.05, **p<0.01

FIG. 25 is a schematic of the tricarboxylic acid cycle (TCA) and exemplary inhibitors of various molecules within the TCA cycle.

### DETAILED DESCRIPTION

This disclosure is based, in part, on the findings that interferon-I is produced during the cytokine storm response by plasmacytoid dendritic cells (pDCs)s in SARS-CoV-2-infected patients. Further, the infiltration of pDCs in the lungs of SARS-CoV-2-infected patients correlated with strong IFN-I signaling in lung macrophages. In patients that developed severe COVID-19, lung macrophages primed by IFN-I express a robust inflammatory signature which correlated at the single cell level with persisting IFN-I signaling. Further, SARS-CoV-2-activated pDCs (SARS pDCs) induced IFN-α-mediated transcriptional and epigenetic mechanisms that lead to the hyperactivation of macrophages by environmental stimuli. Without being bound by theory, it is believed that the uncoupling of the kinetics of the IFN-I signature with the cytokine storm in lung macrophages indicates a link between pDC's response to SARS-CoV-2 and the subsequent macrophage activation in patients with severe COVID-19. Further, the data described points to pDCs, by direct sensing of SARS-CoV-2, and to monocyte-derived macrophages indirectly by phagocytosis of infected cells, as the likely source of IFN-I in the lungs of infected individuals. By promoting the macrophage-induced cytokine storm, this IFN-I response can lead to devastating consequences for a subset of patients.

The Inositol-Requiring Enzyme-X-Box Binding Protein 1 (IRE1α-XBP1 branch of the unfolded protein response (UPR)) inhibits the production of IFN-I by toll-like receptor (TLR)-activated plasmacytoid dendritic cells (pDCs). Mechanistically, IRE1α-XBP1 activation reprograms glycolysis to serine metabolism by inducing phosphoglycerate dehydrogenase (PHGDH) expression. This reduces pyruvate access into the tricarboxylic (TCA) cycle and blunts mitochondrial ATP generation that is necessary for IFN-I production. Furthermore, decreased expression of PHGDH and UPR-controlled genes in pDCs purified from patients with systemic sclerosis (SSc) was observed. Accordingly, pharmacological blockade of tri-carboxylic acid (TCA) cycle reactions can inhibit IFN-I responses in pDCs of patients. These findings link the UPR to metabolic control of pDC hyperactivation and suggest that modulating this process may represent an unconventional strategy for the treatment of viral diseases associated with the cytokine storm.

Thus, this disclosure features agents that activate the UPR response in immune cells such as dendritic cells, and agents that disrupt the TCA cycle in such cells. The disclosure features methods of using such agents to treat a human subject with a viral disease (e.g., COVID-19) and/or to reduce production of inflammatory cytokines or chemokines by immune cells such as DCs (e.g., type I interferon (IFN-I), IL-6, or TNF-α, type III interferon, MIP-1a/CCL3, MIP-1/CCL4, CCL5/RANTES, and IP-10/CXCL10).

A detailed description of the UPR activating agents and the TCA cycle disrupting agents, as well as methods of using these agents are set forth below.

### TCA Cycle Disrupting Agents

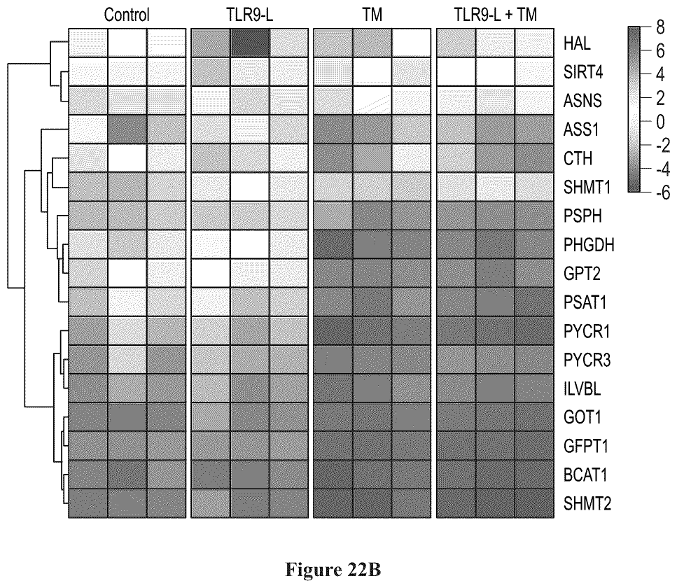

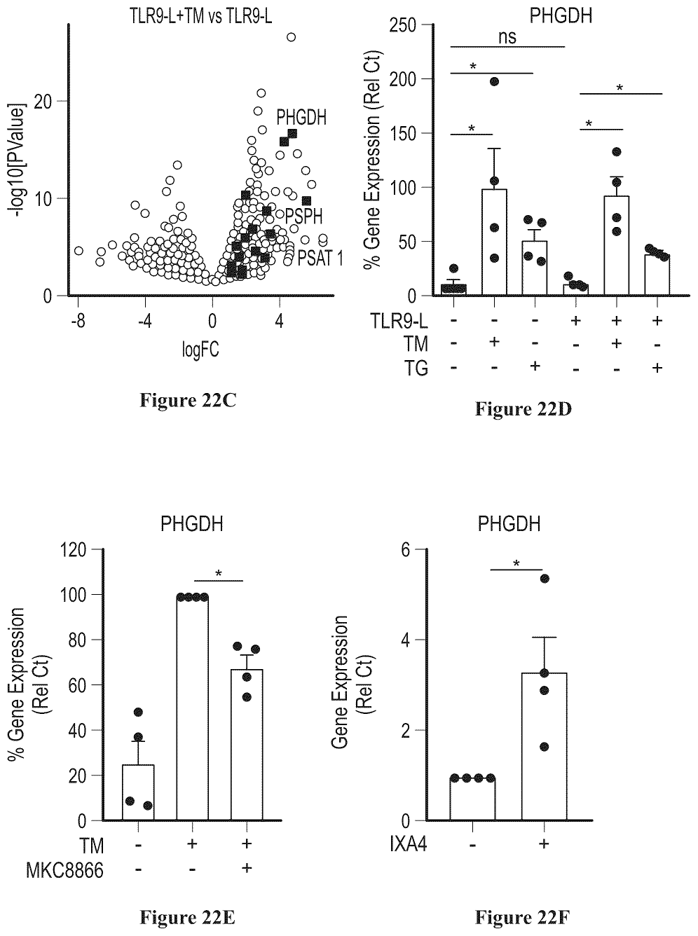

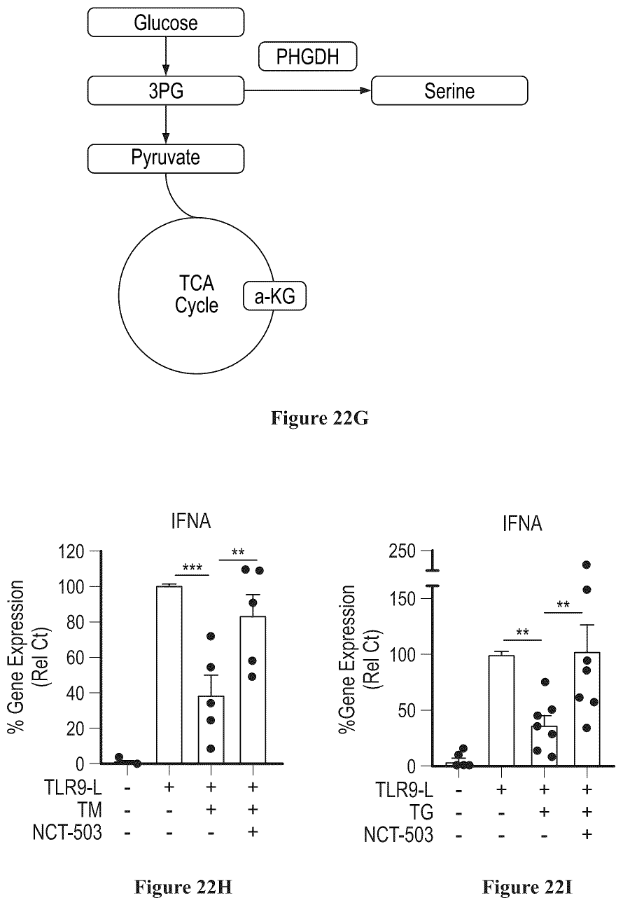

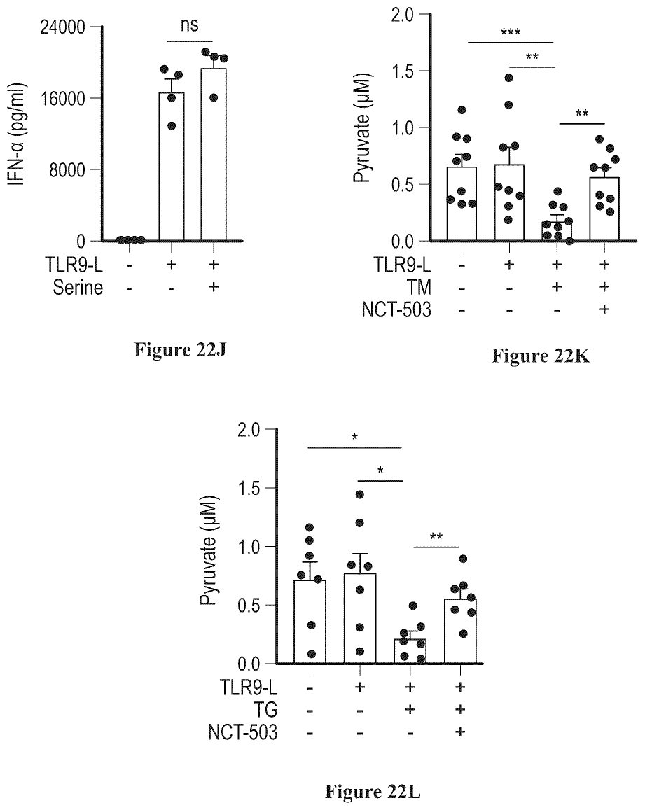

The tricarboxylic acid (TCA) cycle (also called the Krebs cycle) is the second stage of cellular respiration. It is a series of chemical reactions to release stored energy through the oxidation of acetyl-CoA derived from carbohydrates, fats, and proteins. In eukaryotic cells, the citric acid cycle occurs in the matrix of the mitochondrion. In the context of this disclosure, the term “disrupt”, with respect to the TCA cycle disrupting agents refers to agents that inhibit mitochondrial metabolism in cells such as immune cells (macrophages and dendritic cells). The TCA cycle and exemplary inhibitors thereof are shown in FIG. 22.

In some embodiments, the TCA cycle disrupting agent is any of the compounds of Formula I or a pharmaceutically acceptable salt thereof as described in U.S. Pat. No. 9,839,691, incorporated by reference in its entirety. A compound of Formula I has the following structure:

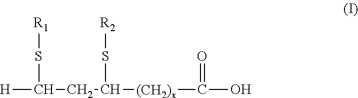

wherein R₁ and R₂ are independently selected from the group consisting of acyl defined as R₃C(O)—, alkyl defined as CₙH₂ₙ₊₁, alkenyl defined as CₘH₂ₘ₋₁, alkynyl defined as CₘH₂ₘ₋₃, aryl, heteroaryl, alkyl sulfide defined as CH₃(CH₂)ₙ—S—, imidoyl defined as R₃C(═NH)—, hemiacetal defined as R₄CH(OH)—S—, and hydrogen provided that at least one of R₁ and R₂ is not hydrogen; wherein R₁ and R₂ as defined above can be unsubstituted or substituted; wherein R₃ is hydrogen, alkyl, alkenyl, alkynyl, cycloalkyl, aryl, alkylaryl, heteroaryl, or heterocyclyl, any of which can be substituted or unsubstituted; wherein R₄ is CCl₃ or COOH; and wherein x is 0-16, n is 0-10 and m is 2-10. In some embodiments, R₁ and R₂ are benzyl or benzoyl. In some embodiments, the the compound of Formula I is

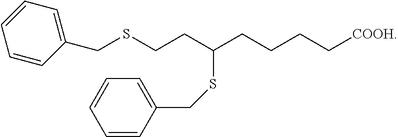 [SMILES: O=C(O)CCCCC(CCSCc1ccccc1)SCc1ccccc1]

In some embodiments, the compound of formula I is 6,8-bis-benzylthio-octanoic acid (CPI-613 or Devimistat).

In some embodiments, the TCA cycle disrupting agent is UK5099 which inhibits mitochondrial pyruvate carrier, a carrier which transport pyruvate from cytoplasm to mitochondria. UK5099 has the following structure:

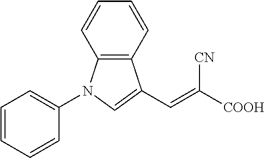 [SMILES: N#C/C(=C\c1cn(-c2ccccc2)c2ccccc12)C(=O)O]

In some embodiments, the TCA cycle disrupting agent is CB839 (Telagenastat), which inhibits glutaminase, an enzyme that converts glutamine to glutamate. CB839 has the following structure:

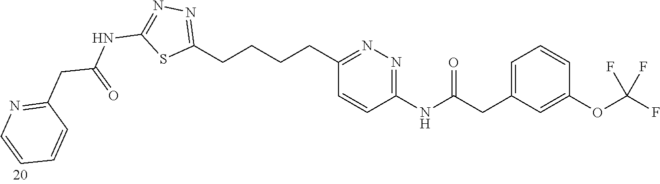 [SMILES: O=C(Cc1cccc(OC(F)(F)F)c1)Nc1ccc(CCCCc2nnc(NC(=O)Cc3ccccn3)s2)nn1]

### Unfolded Protein Response Activating Agents

The unfolded protein response (UPR) is an adaptive response that maintains the fidelity of the cellular proteome in conditions that subvert the folding capacity of the cell, such as those noticed in infection and inflammatory contexts. In immunity, the UPR sensor IRE1 (Inositol-requiring enzyme 1-alpha) is as a critical regulator of the homeostasis of antigen presenting cells (APCs). Flores-Santibinez F, et al. Cells. 2019; 8(12):1563. The IRE1α/XBP1s signaling pathway is an arm of the unfolded protein response (UPR) that safeguards the fidelity of the cellular proteome during endoplasmic reticulum (ER) stress, and that has also emerged as a key regulator of dendritic cell (DC) homeostasis. Medel B. et al., Frontiers in Immunology, 2019(9); Article 3050.

In the context of this disclosure, compounds that activate the UPR in plasmocytoid dendritic cells, particularly the IRE1α/XBP1s signaling pathway, can be used in the methods to treat viral diseases and/or to reduce proinflammatory cytokine production. Such UPR activating agents include, but are not limited to tunicamycin and thapsigargin. As described herein, the term “activates the UPR” refers to the ability of the agent to activate and/or enhance the unfolded protein response, in particular, the IRE1α-XBP1 signaling branch of the UPR in cells (e.g., immune cells such as macrophages and dendritic cells).

Exemplary UPR activating agents that can be utilized in the methods described herein have the structures provided below:

TABLE 1
UPR activating agents and their structures

| Agent | Structure |
|---|---|
| Tunicamycin (NSC 177382) | 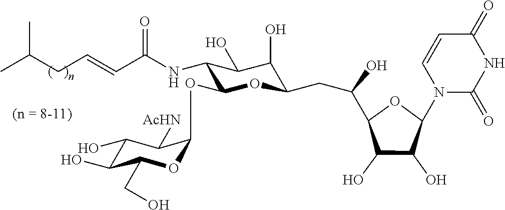 |
| Thapsigargin | 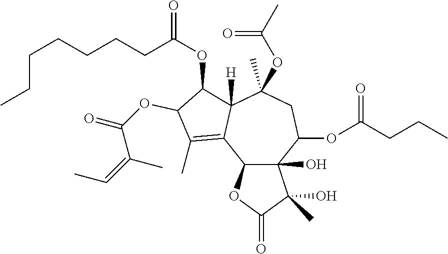 [SMILES: C/C=C(/C)C(=O)OC1C(C)=C2[C@H]([C@@H]1OC(=O)CCCCCCC)[C@@](C)(OC(C)=O)CC(OC(=O)CCC)[C@@]1(O)[C@H]2OC(=O)[C@@]1(C)O] |
| IXA4 | 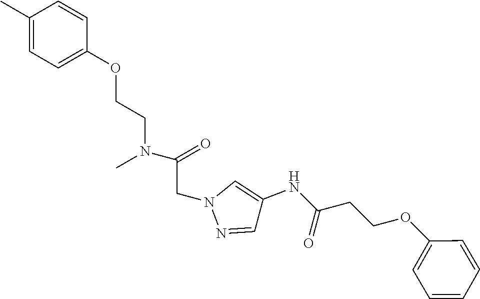 [SMILES: Cc1ccc(OCCN(C)C(=O)Cn2cc(NC(=O)CCOc3ccccc3)cn2)cc1] |

Any of the UPR activating agents shown in Table 1 or analogs thereof can be utilized in the methods of this disclosure.

### Viral Diseases, Immune Cells, and Cytokines

The disclosure features methods of treating viral diseases, in particular viral diseases associated with the cytokine storm or hypercytokinemia. Cytokine storm defines a dysregulation of and an excessively exaggerated immune response often accompanying selected viral infections, such as influenza, SARS (including SARS-CoV-2), MERS, and hantavirus. Cytokine storm is an umbrella term encompassing several disorders of immune dysregulation characterized by constitutional symptoms, systemic inflammation, and multiorgan dysfunction that can lead to multiorgan failure if inadequately treated. See e.g., Fajgenbaum and June N Engl J Med 2020; 383.2255-2273.

Nonspecific markers of inflammation such as C-reactive protein (CRP) are universally elevated and correlate with severity. Other symptoms of a cytokine storm include, but are not limited to hypertriglyceridemia and various blood-count abnormalities, such as leukocytosis, leukopenia, anemia, thrombocytopenia, and elevated ferritin and d-dimer levels. Prominent elevations in serum inflammatory cytokine levels, including but not limited to interferon-γ(or CXCL9 and CXCL10, chemokines induced by interferon-γ), interleukin-6, interleukin-10, and soluble interleukin-2 receptor alpha, a marker of T-cell activation, are usually present in a cytokine storm. Innate cells that are most often implicated in the pathogenesis of cytokine storm include neutrophils, macrophages, and NIK cells.

Serum cytokine levels that are elevated in patients with Covid-19-associated cytokine storm include but are not limited to interleukin-1β, interleukin-6, IP-10, TNF, interferon-γ, macrophage inflammatory protein (MIP) 1α and 1β, and VEGF. In some embodiments, the disclosued methods of this disclosure treat the cytokine storm by reducing levels of these cytokines.

Apart from SARS-CoV-2 infections that cause coronavirus disease (COVID-19), the methods of this disclosure can be used to treat other viral diseases, including, but not limited to influenza Severe Acute Respiratory Syndrome (SARS), and Hantavirus Pulmonary Syndrome (HPS).

In some embodiments, the methods of the disclosure can treat viral conditions which are IFN-I-mediated. The methods of this disclosure may be used to block the TCA cycle and/or activate the UPR in a range of immune cells, including, but not limited to, dendritic cells, macrophages, T cells, B cells, natural killer cells, and/or neutrophils. Several types of immune cells that are involved in the pathology of viral diseases. See e.g., Tang L et al., Front. Immunol., Nov. 30, 2020.

The virus can promote the activation of immune cells (such as T cells, B cells, macrophages, dendritic cells, neutrophils, monocytes) and resident tissue cells, resulting in the production of large amounts of inflammatory cytokines. During the flu virus infection, innate immune responses get started through the cascade amplification reactions of interferon stimulated gene expression, and type I interferon (IFN) is mainly produced by monocytes, macrophages and dendritic cells.

Serum levels of interleukin 8 (IL-8), IP-10 (interferon-induced protein 10), MCP-1 (monocyte chemoattractant protein-1), MIP-1 (macrophage inflammatory protein-1), MIG (monokine induced by IFN-γ) and CXCL-9 (CXC chemokine ligand-9) can be abnormally elevated in H5N1 influenza virus infection, while IL-8, IL-9, IL-17, IL-6, IL-15, TNF-α (tumor necrosis factor-α), IL-10 can be increased in H1N1 influenza virus infection. Earlier researches demonstrated that serum levels of proinflammatory factors IFN-γ, IL1β, IL-6, IL-12, IL-18, IP-10, MCP-1, and CCL2 (CC chemokine ligand-2), CXCL-10 and IL-8 are positively correlated with lung inflammation and extensive lung tissue injury in SARS patients (19-21). Whereas, the levels of serum pro-inflammatory cytokines IL-6, IFN-γ, TNF-α, IL-15, IL-17, and chemokines IL-8, CXCL-10, and CCL5 were significantly increased in severe MERS patients (22, 23). Among numerous molecules that increase in virally-mediated cytokine storms, IL-6, IFN-γ, IL-1β, IL-8, IL-10, and TNF-α are of crucial importance (9, 24, 25). The occurrence of cytokine storm has been reported to be one of the main causes of death in patients with SARS-CoV, MERS-CoV, and influenza virus infections (8, 26). Similarly, cytokine storm is also a common feature of severe cases in COVID-19, and elevated levels of serum IL-6 and CRP correlate with respiratory failure, ARDS, MOF and adverse clinical outcomes (27, 28).

Plasmocytoid dendritic cells (pDCs) are danger-sensing cells that produce interferon (IFN)—I. IFNs are generally classified into three families—IFN-I, IFN-II and IFN-III—which differ in their immunomodulatory properties, their structural homology and the group of cells from which they are secreted [3, 4]. IFN-Is (IFN-α, -β, -ω, -ε, -κ) compose the largest family and, alongside IFN-III (IFN-λ), activates intracellular signaling pathways that mediate immune responses against viruses and tumors. (Psarras A et al., Rheumatology, Volume 56, Issue 10, October 2017, Pages 1662-1675).

pDCs play a crucial role in antiviral immunity and are regarded as precursor DC which are effectively interferon producing cells. In some embodiments, the methods of the disclosure can be used to modulate signaling pathways in pDCs and other immune cells, thereby treating viral conditions. In some embodiments, the pDCs that are modulated are dendritic cells express one or more of CD123, CD303 (BDCA2), CD304 (BDCA4), and immunoglobulin-like transcript 7 (ILT7), but do not express the lineage-associated markers (Lin) CD3, CD19, CD14, CD16 and CD11c. See, e.g., Ye, Y, et al., Clinical & Translational Immunology (2020); 9: e1139; Tang F, Du Q, Liu Y J. Sci China Life Sci. 2010 February; 53(2):172-82; Gilliet M, Cao W, Liu Y J. Nat Rev Immunol. 2008 August; 8(8):594-606; and Barrat F. J. and Su L., J Exp Med. 2019 Sep. 2; 216(9):1974-1985 for a review of pDCs, characterization of these cells, and their role in viral conditions.

### Additional Treatments

This disclosure features combination therapies wherein the UPR activating agent and/or the TCA cycle disruptor is administered with one or more additional treatments. The additional treatment can be an art-recognized therapy for a viral diseases (e.g., COVID-19). See Kheirabadi D, et al., J Med Virol. 2021; 93(5):2705-2721. doi:10.1002/jmv.26811. Treatments for viral disease include, but are not limited to, a corticosteroid, remdesivir, Nirmatrelvir, Bebtelovimab, Molnupiravir, an IL-6 inhibitor, an IL-1 inhibitor, a kinase inhibitor, a complement inhibitor, ivermectin, hydroxychloroquine, favipiravir, interferon-beta and a nonsteroidal anti-inflammatory drug (NSAID). Immunosuppressant treatment includes, but is not limited to, treatment with methotrexate, mycophenolate mofetil (MMF), cyclophosphamide, cyclosporin, or azathioprine. The corticosteroid that can be used as an additional treatment includes, but is not limited to, dexamethasone, prednisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

The anti-inflammatory cytokine that can be used as an additional treatment includes, but is not limited to, interleukin (IL)-1 receptor antagonist, IL-4, IL-6, IL-10, IL-11, and IL-13. Specific cytokine receptors for IL-1, tumor necrosis factor-alpha, and IL-18 also function as pro-inflammatory cytokine inhibitors. The nature of anti-inflammatory cytokines and soluble cytokine receptors are known in the art and discussed in Opal and DePalo, Chest, 117(4): 1162-72 (2000).

In some embodiments, the additional therapy includes one or more of: sulfasalazine, doxycycline, minocycline, penicillamine, tofacitinib, and leflunomide.

The components of the combination therapy may be administered substantially at the same time or sequentially.

### Methods of Treatment

The disclosure features a variety of methods for treating a viral disease or condition, (e.g., COVID-19) using the agents described herein.

As used herein, the term “treat” “treatment,” or “treating” a subject having an viral condition, are used in connection with a given treatment for a given disorder, wherein at least one symptom of the disorder is alleviated, or ameliorated. The treatment may inhibit deterioration or worsening of a symptom of the disclosed conditions (e.g., COVID-19) or may cause the condition to develop more slowly and/or to a lesser degree (e.g., fewer symptoms in the subject) in the subject than it would have absent the treatment. A subject is treated with the methods of this disclosure, to improve a condition, symptom, or parameter associated with a disorder or to prevent progression or exacerbation of the disorder (including secondary damage caused by the disorder) to either a statistically significant degree or to a degree detectable to one skilled in the art. A subject who is at risk for, diagnosed with, or who has one of the viral diseases and/or cytokine storn of this disclosure can be administered a compound of this disclosure (e.g., an agent that activates the UPR and/or an agent that disrupts the TCA cycle) in an amount and for a time to provide an overall therapeutic effect. A compound of this disclosure can be administered alone (monotherapy) or in combination with other agents (combination therapy), which agents are described in the “Additional treatments” section.

As used herein, the term “therapeutically effective amount” of an agent is an effective amount that may be determined by the effect of the administered agents or the combined effects of the agents (if more than one agent is used). The “therapeutically effective amount” of the agent of this disclosure is an amount that results in a reduction in the severity of disease symptoms, the frequency and length of periods without disease symptoms. Preferably, it results in prevention of dysfunction or disability due to an increase in disease or distress. For example, in the case of COVID-19, a therapeutically effective amount can be, for example, one that prevents or treats the cytokine storm. It is preferable to prevent further deterioration of physical symptoms associated with COVID-19. A therapeutically effective amount is also preferred to prevent or delay the onset of the cytokine storm, as may be desired when early or preliminary signs of disease (COVID-19) are present. Similarly, delaying the chronic progression associated with COVID-19 is also desired. Any clinical or biochemical test that monitors the above can be used to determine whether a particular treatment is in a therapeutically effective amount to treat COVID-19. Those skilled in the art will be able to determine such amounts based on factors such as the size of the subject, the severity of the subject's symptoms, and the particular composition or route of administration chosen.

The disclosed methods can also treat or prevent a COVID-19 cytokine storm by reducing the level of IL-Ib and/or TNF-a in the (blood or plasma) of the subject, preferably reducing the level of one of more, two or more, three or more, four or more, five or more or all six or TNF-a, IL-1B, IP-10, IL-6, IL-8, MCP-1, MIP-1a and MIP-Ib in (the blood or plasma) of the subject. The SARS-CoV-2 to be treated may either be the original wild-type strain and/or one or more variants. Examples of variants of SARS-CoV-2 that can be treated include, but are not limited to, the variants D614G, B.1.351, B.1.1.7, PI, P2, B.1.617, B.1.427, B.1.429, B.1.525 and B.1.526.

The therapeutically effective amount of the agent may also vary according to factors such as the disease state, the age, sex, and weight of the individual, and the ability of the compound to elicit a desired response in the individual, e.g., to ameliorate at least one parameter of the condition or to ameliorate at least one symptom of the condition. A therapeutically effective amount is also an amount where the therapeutically beneficial effect exceeds any toxic or detrimental effect of the composition. A therapeutically effective amount of an agent of this disclosure (i.e., an effective dosage) includes milligram, microgram, nanogram, or picogram amounts of the agent per kilogram of subject or sample weight (e.g., about 1 nanogram per kilogram to about 500 micrograms per kilogram, about 1 microgram per kilogram to about 500 milligrams per kilogram, about 100 micrograms per kilogram to about 5 milligrams per kilogram, or about 1 microgram per kilogram to about 50 micrograms per kilogram).

The amounts and times of administration for combination therapies can be those that provide, e.g., an additive or a synergistic therapeutic effect. Further, the administration of the compound of this disclosure (e.g., a UPR activator and/or a TCA cycle inhibitor) can be used as a primary, e.g., first line treatment, or as a secondary treatment, e.g., for subjects who have an inadequate response to a previously administered therapy (i.e., a therapy other than one with a compound of this disclosure). In some embodiments, the combination therapy includes the use of a compound of this disclosure with one or more of the following agents: a corticosteroid, remdesivir, Nirmatrelvir, Bebtelovimab, Molnupiravir, an IL-6 inhibitor, an IL-1 inhibitor, a kinase inhibitor, a complement inhibitor, ivermectin, hydroxychloroquine, favipiravir, interferon-beta and a nonsteroidal anti-inflammatory drug (NSAID).

As used herein, the term “control” refers to an age-matched subject that does not have or is not diagnosed with a viral condition. In some embodiments, a control refers to an age-matched and sex-matched subject that is not treated with the method of this disclosure, or is treated with a placebo. In some embodiments, a control refers to a population average for the amount or degree of a particular parameter in a normal healthy population.

Treatment outcomes on viral diseases can be measured using any of the routine assays and techniques known in the art, including but not limited to enzyme-linked immunosorbent assay (ELISA), multiplex cytokines assay (Aziz N. Immunopathol Dis Therap. 2015; 6(1-2):19-22), qualitative and quantitative polymerase chain reaction (PCR), and patient-reported outcome measures. Clinical outcomes can be measured using several clinical features such as those described in Touma, Zahi (Ed.) Outcome Measures and Metrics in Systemic Lupus Erythematosus; Pages 1-50.

The methods of the disclosure can reduce production of inflammatory cytokines or chemokines by immune cells (such as dendritic cells) in the human subject. In some embodiments, the methods of this disclosure reduce IFN-I production in the human subject in need thereof by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, as compared to the corresponding reference levels in the human subject or in a control. The methods can also reduce the expression of interferon stimulated genes, including, but not limited to, Guanylate Binding Protein 1 (GBP1), Interferon Regulatory Factor 7 (IRF7), interferon stimulated gene 54 (ISG54), myxovirus resistance protein B (MxB), and 2′-5′-Oligoadenylate Synthetase 2 (OAS2). The methods can also enhance expression of phosphoglycerate dehydrogenase (PHGDH), phosphoserine Phosphatase (PSPH), and phosphoserine Aminotransferase 1 (PSAT1). In some cases, the methods of the disclosure can reduce CXCL4 expression in DCs.

### EXAMPLES

The practice of the methods and compositions of the disclosure employs, unless otherwise indicated, conventional techniques of molecular biology (including recombinant techniques), cell culture, immunology, cell biology, and biochemistry, which are well within the purview of the skilled artisan. Such techniques are explained in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the methods and compositions of the disclosure. Particularly useful techniques for particular embodiments will be discussed in the sections that follow. The materials, reagents, and methods, further described below, are used in the following examples. The invention, as described in the following examples, do not limit the scope of the invention described in the claims.

### Materials & Methods

### Purification of Cells

Enriched leukocytes were obtained from the New York Blood Center (Long Island City, NY) after informed consent of donors who were deemed healthy by the New York Blood Center's criteria and used under a protocol approved by the Institutional Review Board of the Hospital for Special Surgery and the Institutional Biosafety Committee of Weill Cornell Medicine. The clinical and demographic characteristics of the donors reflect the diverse population of New York City. PBMCs were prepared using Ficoll-Paque density gradient (GE Healthcare) as previously described (32). pDCs and monocytes were isolated from PBMCs by positive selection using BDCA4-conjugated (33) and CD14-conjugated microbeads (Miltenyi Biotec), respectively. pDC depleted PBMC were prepared by removing BDCA4-positve cells from PBMC using microbeads (Miltenyi Biotec). Macrophages were differentiated from monocytes by culturing in complete RPMI 1640 medium with 20 ng/ml M-CSF for 1-5 d.

### Primary Human Pulmonary Cells

Human alveolar epithelial cells were obtained from ScienceCell and cultured in Alveolar Epithelial Cell Medium (ScienceCell) and expanded in T-25 plates and plated in Transwell plates (Corning) for additional experiments. Human alveolar macrophages were obtained from Accegen and were cultured in complete RPMI 1640 medium with 100 U/mL M-CSF, 1 mM Sodium Pyruvate, 10 mM HEPES, and 1× penicillin/streptomycin.

hPSC Culture The human induced pluripotent stem cells (iPSCs) were grown and maintained on 1% Matrigel-coated six-well plates in mTeSR (Stem Cell Technology) with 5% CO2 culture condition. The medium was changed daily. When iPSCs reached ˜90% confluence, the cells were passaged at 1:6-1:12 with Accutase (Stem Cell Technology).
hPSC Differentiation

Human induced pluripotent stem cells were differentiated into monocytes and macrophages as previously reported56. In brief iPSC were treated with accutase and scraped to produce small cellular groups and replated onto 1% Matrigel-coated 6-well plates. After 1-2 days, mTeSr medium was replaced with Mesoderm induction medium containing 15 ng/ml Activin A (R&D Systems), 40 ng/ml BMP-4 (R&D Systems), and 1.5 μm CHIR99021 (Stem Cell Technologies). On Day 2 the medium was replaced with hemogenic endothelium induction containing 50 ng/ml IL-6 (R&D Systems), 15 ng/ml IL-3 (R&D Systems), 50 ng/ml TPO (R&D Systems), 12.5 ng/ml bFGF (Peprotech), 50 ng/ml SCF (R&D Systems), and 50 ng/ml VEGF (R&D Systems). On Day 5 the medium was replaced with hematopoietic induction medium containing 50 ng/ml VEGF (R&D Systems), 50 ng/ml bFGF, 50 ng/ml SCF (R&D Systems) and 10 μM SB431542 (Tocris). On day 9 the cells were dissociated with Accutase and resuspended with Monocyte induction medium containing 50 ng/ml IL-6 (R&D Systems), 12 ng/ml IL-3 (R&D Systems), and 80 ng/ml M-CSF (R&D Systems) into low attachment plates. On day 15, floating cells were collected and StemSep Human CD14 Positive Selection Kit (Stem Cell Technologies) was used to isolated CD14+ cells. Macrophage were obtained by plating monocytes onto FBS-coated plates with macrophage differentiation media containing M-CSF. Cells were cultured under normoxic conditions at 37° C. and 5% CO2.

Activation of Cells For functional assay, freshly isolated pDCs were resuspended at 3.104 cells/100 ml complete RPMI and cultured in 96 wells/U bottom plate. PBMC and pDC-depleted PBMC were resuspended at 3×10⁵ cells/100 ml complete RPMI and cultured in 96 wells/flat bottom plate. Untreated and gamma-irradiated inactivated SARS-CoV-2 (USA-WA1/2020, BEI resources) were used at a multiplicity of infection (MOI) of 1, 0.25, 0.5, 0.1 and 0.01 whereas Flu virus was used at MOI 2 for 3, 6, 10 and 18 hours. For blocking experiment, cells were pre-incubated for 1 h with the ACE2 inhibitor (Novus biologicals, 2 μM), TLR7 inhibitor IRS661 (2 μM), PI3Kδ inhibitor CAL-101 (10 μM), chlathrin inhibitor chlorpromazine (CPZ, 30 μM) or dynamin inhibitor dynasore hydrate (DH, 100 μM) followed by the addition of inactivated SARS-CoV-2 at MOI 0.25. After differentiation of macrophages, the cells were cultured at 1.105 cells/100 ml complete RPMI for 24 h either with 10% of non-activated pDC supernatant (Unst-pDC SN), 10% of SARS-CoV-2 activated pDC supernatant (SARS-pDC SN), recombinant IFN-α at 100000, 30000, 10000, 3000, 1000 and 300 μg/ml (PBL assay science) or inactivated SARS-COV-2 (MOI 0.25). LPS (2 or 10 ng/ml), Pam3Cys (20 ng/ml), PolyIC (10 ng/ml), ORN8L (60 μg/ml) or inactivated SARS-COV-2 (MOI0.25) were added in the culture for 6 h. For blocking experiments, macrophages were preincubated with JAK1/2 inhibitor baricitinib (2 μM) or anti-IFNAR antibody (2 μg/ml) for 1 h before adding of SARS-pDC SN for 24 h. TLR ligands were then added to the well for 6 h. For TNF blocking, SARS-pDC SN and macrophages were pre-incubated for 1 hour with anti-hTNF (10 μg/ml, R&D Systems) and anti-hTNFRI (10 μg/ml, R&D Systems) respectively. After 1 hour, macrophages were incubated with SARS-pDC SN+ anti-hTNF for 24 hours. LPS (10 ng/ml) was then added to the well for 6 h.

### Primary Human Pulmonary Cell Co-Culture

2×10⁵ human alveolar epithelial cells were plated in regular Tissue Culture plates in Alveolar Epithelial Cell Medium supplemented with 100 U/mL M-CSF while 5.104 human alveolar macrophages were plated in Transwell Plate Inserts in Alveolar Epithelial Cell Medium supplemented with 100 U/mL M-CSF. Cultures were inoculated with MOCK infection or SARSCoV-2 (USA-WA1/2020, BEI resources) at MOI=0.1. After infections cells were lysed in Trizol or fixed in 4% paraformaldehyde for 24 hrs. For co-culture cells, cells were lifted with trypsin and then fixed in 4% paraformaldehyde for 24 hrs. Macrophages were then isolated and separated used anti-human CD68 antibody (Stem Cell Technology) and StemSep beads (Stem Cell Technology).

### SARS-CoV-2 Propagation and Infection

SARS-CoV-2 isolate USA-WA1/2020 (NR-52281) was provided by the Center for Disease Control and Prevention and obtained through BEI Resources, NIAID, NIH. SARS-CoV-2 was propagated in Vero E6 cells in DMEM supplemented with 2% FBS, 4.5 g/L D-glucose, 4 mM Lglutamine, 10 mM Non-essential amino acids, 1 mM sodium pyruvate and 10 mM HEPES using a passage-2 stock of virus as described previously57. Three days after infection virus-containing supernatants were purified as described previously (Nilsson-Payant, B. E. et al. J Virol 95, e0125721). In brief, supernatant containing propagated virus was filtered through an Amicon Ultra 15 (100 kDa) centrifugal filter (Millipore Sigma) at ˜4000 rpm for 20 minutes. Flow through was discarded and virus was resuspended in DMEM supplemented as described above. Infectious titers of SARS-CoV-2 were determined by plaque assay in Vero E6 cells in Minimum Essential Media supplemented with 2% FBS, 4 mM Lglutamine, 0.2% BSA, 10 mM HEPES and 0.12% NaHCO₃ and 0.7% agar. All MOIs were based on titer determined from plaque assays on Vero E6 cells. All work involving live SARS-CoV-2 was performed in the CDC and USDA-approved BSL-3 facility of the Icahn School of Medicine at Mount Sinai and NYU Langone in accordance with institutional biosafety requirements.

### Real-Time Quantitative PCR Analysis

PCR reactions were performed as described previously with 10 ng of cDNA. In brief, RNA was extracted from cells using the Qiagen RNeasy Mini Kit. Quantity of RNA was measured by Nanodrop, and high-capacity cDNA Reverse Transcription kit was used to generate 20-50 ng cDNA. Gene expression levels were calculated based on relative threshold cycle (Ct) values. This was done using the formula Relative Ct=100×1.8 (HSK-GENE), where HSK is the mean CT of duplicate housekeeping gene runs (Ubiquitin), GENE is the mean CT of duplicate runs of the gene of interest, and 100 is arbitrarily chosen as a factor to bring all values above 0. Primers were from Fischer Scientific.

### Real-Time Quantitative PCR Analysis for Viral Genes

Total RNA samples were prepared from cells using TRIzol and the Direct-zol RNA Miniprep Plus kit (Zymo Research) according to the manufacturer's instructions. To quantify viral replication, measured by the accumulation of subgenomic N transcripts, one-step quantitative real-time PCR was performed using SuperScript III Platinum SYBR Green One-Step qRT-PCR Kit (Invitrogen) with primers specific for the TRS-L and TRS-B sites for the N gene as well as 18S and ACTB as an internal reference as previously described59. Quantitative real-time PCR reactions were performed on the Biorad CFX384 Touch Real-Time PCR Detection System. Delta-delta-cycle threshold (DDCT) was determined relative to the 18S and ACTB and mock infected/treated samples.

### Chemokine and Cytokine Measurement

Supernatant from pDCs 24 h culture was used for quantification of secreted TNF and IL-6 and measured with the use of an enzyme-linked immunosorbent assay (ELISA) according to the manufacturer's protocol (Mabtech).

### Western Blot

Cells were lysed in 50 μl of cold 50 mM Tris-HCl pH 7.4, 150 mM NaCl, 1 mM EDTA, 1% (vol/vol) Triton X-100, 2 mM Na3VO4, 1× phosSTOP EASYPACK, 1 mM Pefabloc, and 1×EDTA-free complete protease inhibitor cocktail (Roche, Basel, Switzerland), and incubated for 10 min on ice. Then, cell debris was pelleted at 13,000 rpm at 4-C for 10 min. The soluble protein fraction was mixed with 4× Laemmli Sample buffer (BIO-RAD, Cat. #1610747) and 2-mercroptoehanol (BME) (Sigma-Aldrich). Samples for Western Blot were subjected to electrophoresis on 4-12% Bis-Tris gels (Invitrogen). To detect IRF5 dimers, Novex WedgeWell 14% Tris-Glycine Gel (Invitrogen, Cat. #XP00140BOX) was adopted to electrophoresis of protein samples according to manufacturer's instruction. Proteins were transferred to polyvinylidene difluoride membrane and immunodetection was performed as previously published 87. Antibodies used are from Cell Signaling Technology: IkB-α (9242s), phosphor-p38 (9215S), p38 (9212S) and Phospho-p44/42 μMAP Kinase (Erk1/2) (9101S).

### Formaldehyde-Assisted Isolation of Regulatory Elements (FAIRE) Assay

To evaluate inflammatory gene in the genome associated with regulatory activity, FAIER assay was adopted as previous study60. Briefly, cells were cross-lined with 1% formaldehyde for 15 min and quenched with 0.125 μM glycine. Then, cells were lysed and sonicated. 10% of the samples were used for input and the rest for phenol/chloroform extraction. The input DNA and extracted DNA were used for qPCR.

### RNA Sequencing

After RNA extraction, libraries for sequencing were prepared using the NEBNext® Ultra™ II RNA Library Prep Kit for Illumina® following the manufacturer's instructions (Illumina). Quality of all RNA and library preparations were evaluated with BioAnalyser 2100 (Agilent) and the sequencing input was 500 ng total RNA. Sequencing libraries were sequenced by the Genomics Facility at Weill Cornell using a NextSeq2000, 50-bp paired-end reads at an average depth of 35 million reads per sample.

### RNA-Seq Analysis

Read quality was assessed and adapters trimmed using fastp. Reads were then mapped to the human genome (hg38) and reads in exons were counted against Gencode version 33 (Ensembl 99) with STAR Aligner. Differential gene expression analysis was performed in R (4) using edgeR. Genes with low expression levels (<4 counts per million in at least one group) were filtered from all downstream analyses. Benjamini-Hochberg false discovery rate (FDR) procedure was used to correct for multiple testing. Downstream analyses were performed in R using a visualization platform built with Shiny developed by bioinformaticians at the David Z. Rosensweig Genomics Research Center at the Hospital for Special Surgery.

Analysis of Single-Nucleus RNA Sequencing Data from Lungs of COVID-19 Patients

Publicly available data of single-nucleus RNA sequencing (snRNA-seq) was used from postmortem lung tissue of COVID-19 patients4. Both the original global UMAP representation and cell type labels provided by authors were used. Since pDCs were not labeled as a cell type, dendritic cells were re-clustered by re-calculating a neighbor graph and computing a UMAP representation with only those cells using default parameters of Scanpy (version 1.8.1), as described (Melms, J. C. et al. Nature 595, 114-119, (2021). Dendritic cells were then clustered using the Leiden algorithm with resolution 0.4. The top representative genes for each cluster were obtained using the “rank_genes_groups” function of Scanpy on the original raw expression values and with a T-test overestimating the variance of each group. Gene expression displayed as overlay to UMAP plots or in heatmaps represents raw counts log transformed after adding the unit value. To test the under- or over-representation of cells of either Control or COVID-19 patients within each cluster, Fisher's exact test was used with a two-sided alternative and multiple testing correction using Benjamini-Hochberg's false discovery rate method in Pingouin (version 0.4.0), as described4.

To investigate the cellular state of each cell type dependent on COVID-19 infection, two sources of gene expression signatures were used: i) genes up-regulated in association with inflammation and fibrotic phenotype in COVID-19 discovered using bulk RNA sequencing; ii) the set of 50 Hallmark pathways from the Molecular Signatures database (MsigDB, version 7.4), as described4.

Each signature was scored in every single cell using the “score_genes” function of Scanpy, and the values were aggregate per cell type and disease state (Control/COVID-19) using the mean. The difference between the values in COVID-19 compared with Control in each cell type was used as a measure of differential signature activity associated with disease. Furthermore, to estimate the relationship between interferon alpha, inflammation and fibrosis during COVID-19, the Pearson correlation was calculated between these signatures across cells of the same cell type and disease state, for all cell types. This was done with equal cell numbers per cell type in Control and COVID-19 by randomly sampling the same number of cells.

### Statistical Analysis

Graphpad Prism for Windows was applied for all statistical analysis. The data are shown as means±SME. Detailed information about statistical analysis, including tests and values used, and number of times experiments is provided in the figure legends.

### Example 1. The Cytokine Storm Produced by Macrophages is Associated with IFN-Induced Signaling in Lungs of Patients with COVID-19

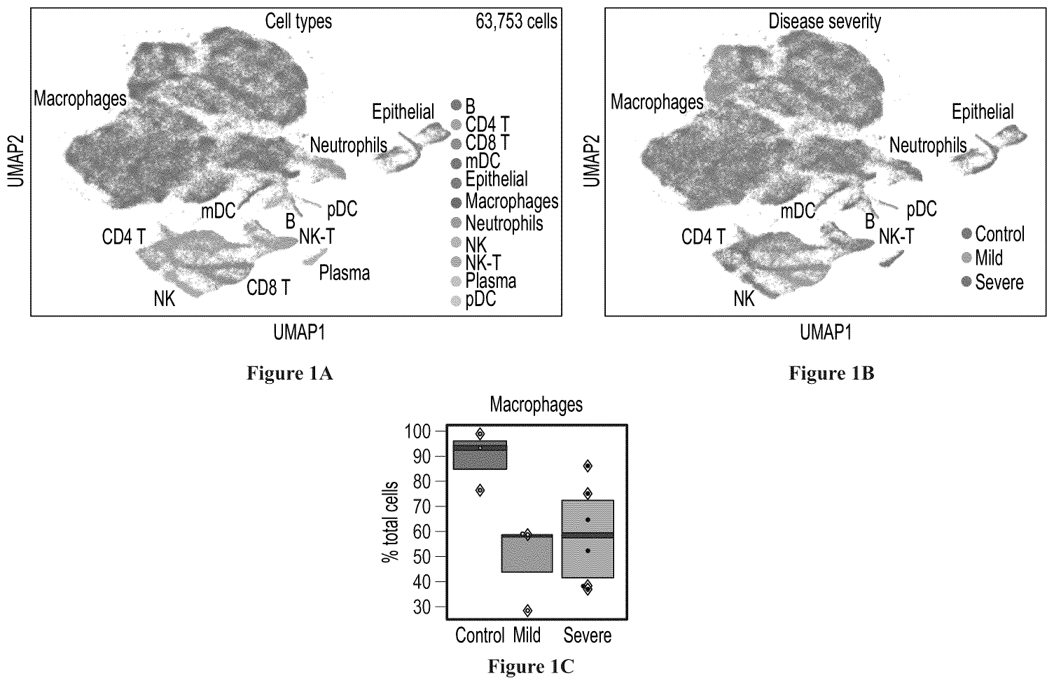

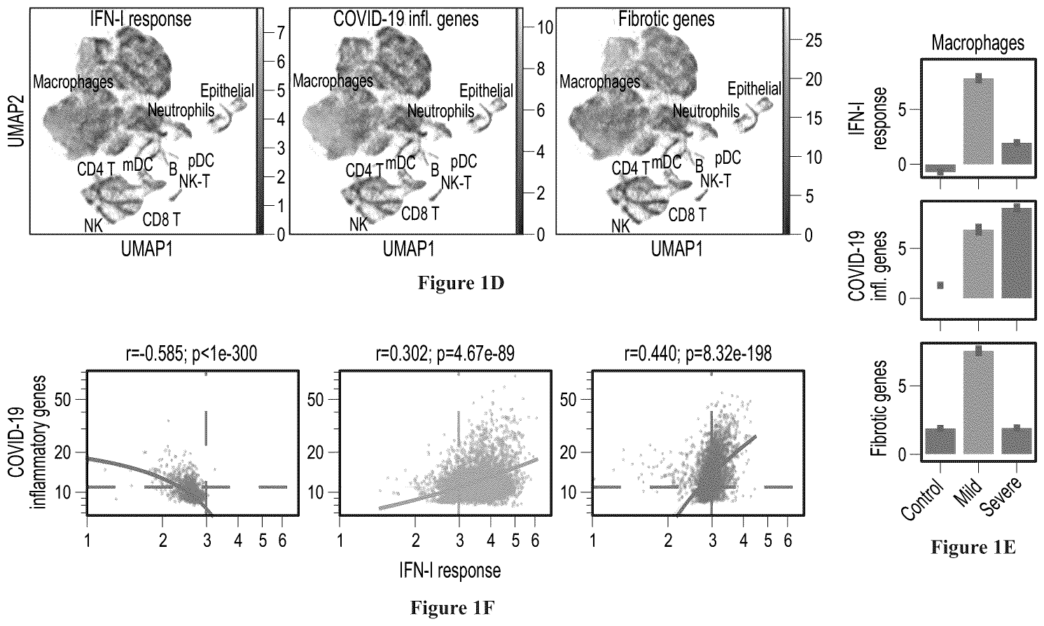

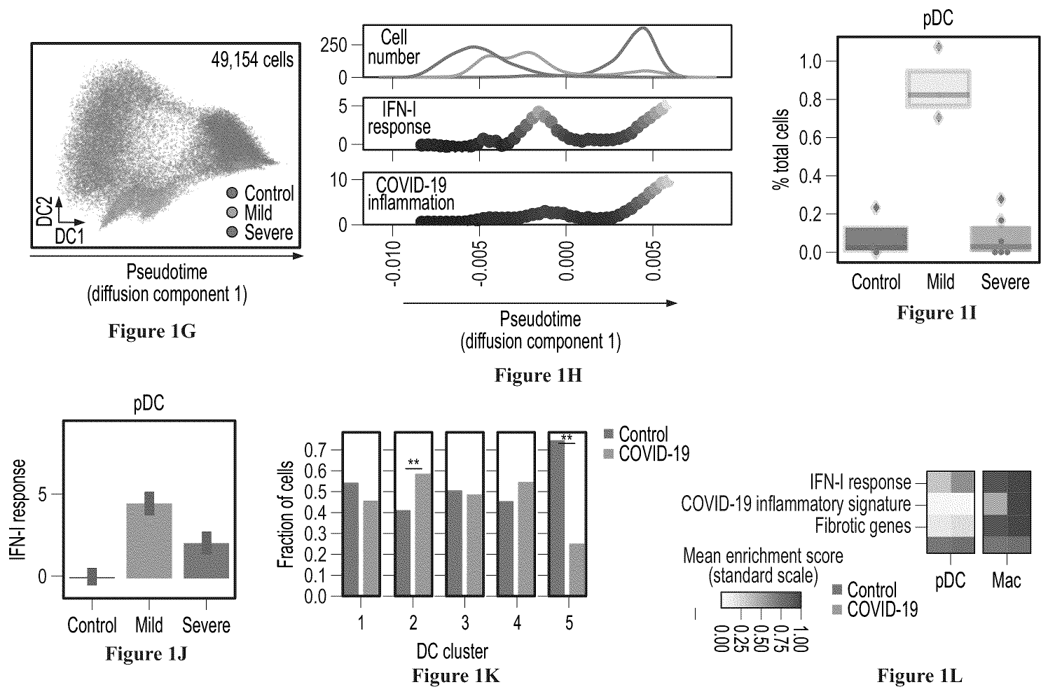

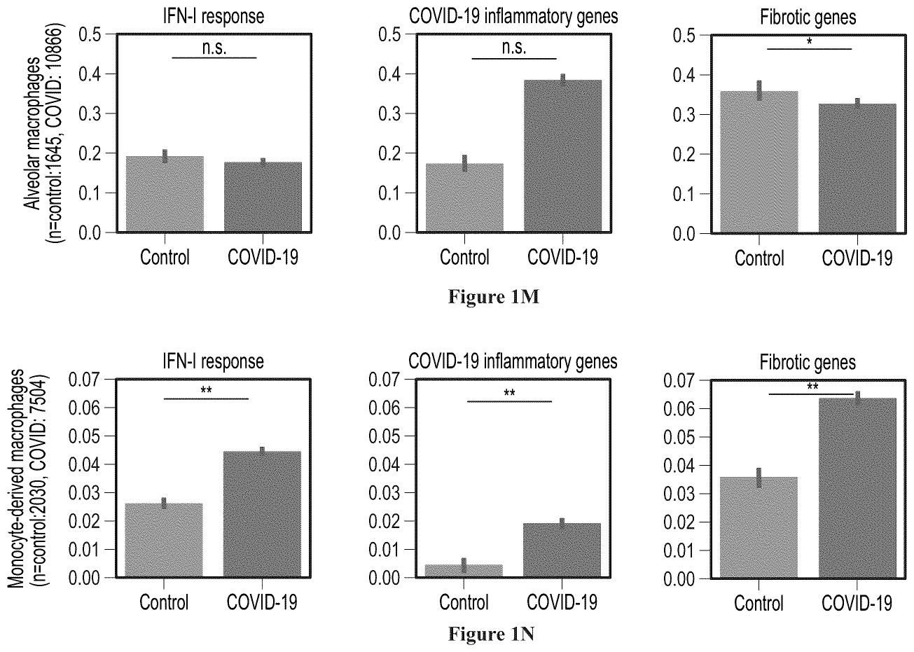

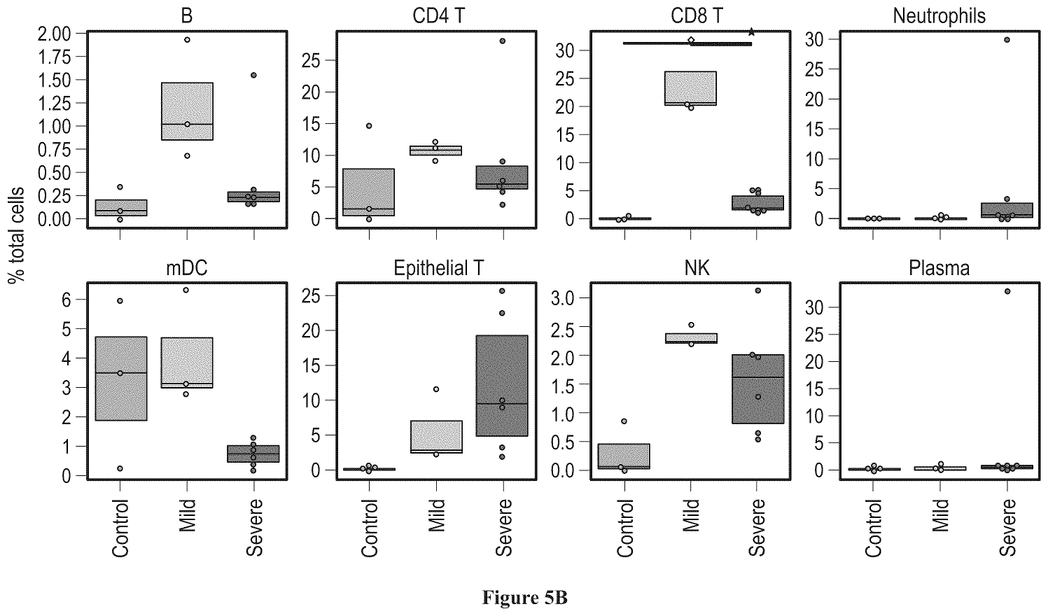

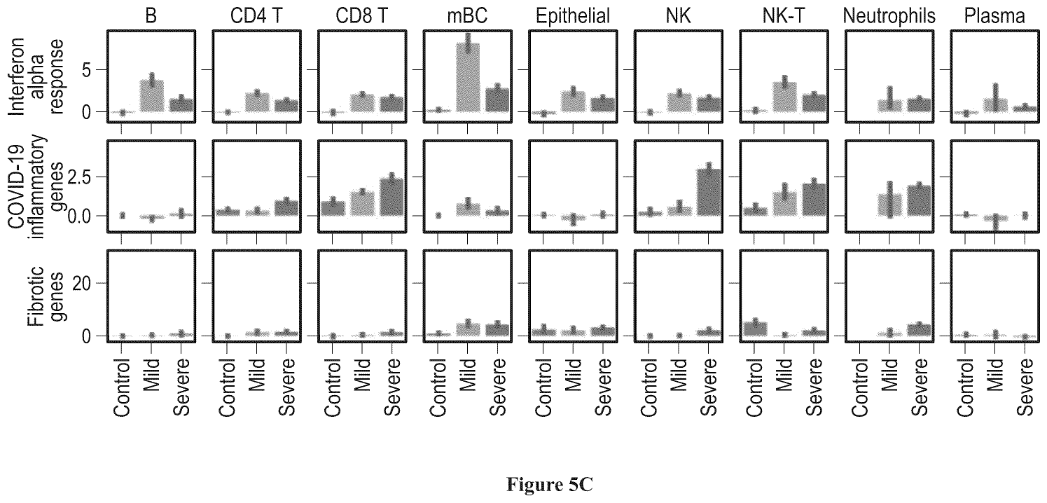

Lung samples were analyzed from patients with terminal COVID-19 (Melms, J. C. et al. Nature 595, 114-119, (2021); Rendeiro, A. F. et al. Nature 593, 564-569, (2021) and cells from bronchoalveolar lavage (BAL) of patients with mild or severe forms of COVID-19 were also analyzed. In this latter population, a large population of macrophages (FIGS. 1A-1C and FIG. 5A-5B) were observed which segregated by disease status (FIG. 1B) but with comparable abundance between patients with mild or severe disease (FIG. 1C). In macrophages from mild patients, a strong IFN-I response signature expression was observed (FIGS. 1D-1E and FIG. 5C), while the IFN-I was reduced in macrophages from patients with severe COVID-19 (FIG. 1E). In contrast, an inflammatory gene signature associated with COVID-19 was seen to be very pronounced in macrophages of patients with severe disease (FIGS. 1D-1E).

TABLE 1
Genes and related functions

| Function | Gene name |
|---|---|
| Interferon | ADAR, B2M, BATF2, BST2, C1S, CASP1, CASP8, CCRL2, CD47, CD74, |
| alpha | CMPK2, CMTR1, CNP, CSF1, CXCL10, CXCL11, DDX60, DHX58, |
| response | EIF2AK2, ELF1, EPSTI1, GBP2, GBP4, GMPR, HELZ2, HERC6, HLA-C, |
| | IFI27, IFI30, IFI35, IFI44, IFI44L, IFIH1, IFIT2, IFIT3, IFITM1, IFITM2, |
| | IFITM3, IL15, IL4R, IL7, IRF1, IRF2, IRF7, IRF9, ISG15, ISG20, LAMP3, |
| | LAP3, LGALS3BP, LPAR6, LY6E, MOV10, MVB12A, MX1, NCOA7, NMI, |
| | NUB1, OAS1, OASL, OGFR, PARP12, PARP14, PARP9, PLSCR1, PNPT1, |
| | PROCR, PSMA3, PSMB8, PSMB9, PSME1, PSME2, RIPK2, RNF31, |
| | RSAD2, RTP4, SAMD9, SAMD9L, SELL, SLC25A28, SP110, STAT2, |
| | TAP1, TDRD7, TENT5A, TMEM140, TRAFD1, TRIM14, TRIM21, |
| | TRIM25, TRIM26, TRIM5, TXNIP, UBA7, UBE2L6, USP18, WARSI |
| Fibrotic | CACNA1A, CD40, CEBPB, CREBBP, EDN1, EZH2, FTH1, ITGB7, ITGB8, |
| genes | MYL12A, MYL12B, PDGFA, PDGFB, PRKCE, RAP1B, RHOH, SMAD3, |
| | VEGFA, WNT5B |
| COVID-19 | CCL15, CCL20, CCL3, CCL4, CCL5, CCL7, CCL8, CTSD, CXCL2, CXCL3, |
| related | CXCL9, IL12B, IL15, IL18, IL1A, IL1B, IL23A, IL6, IL7, S100A12, S100A8, |
| inflammatory | TNF |
| genes | |

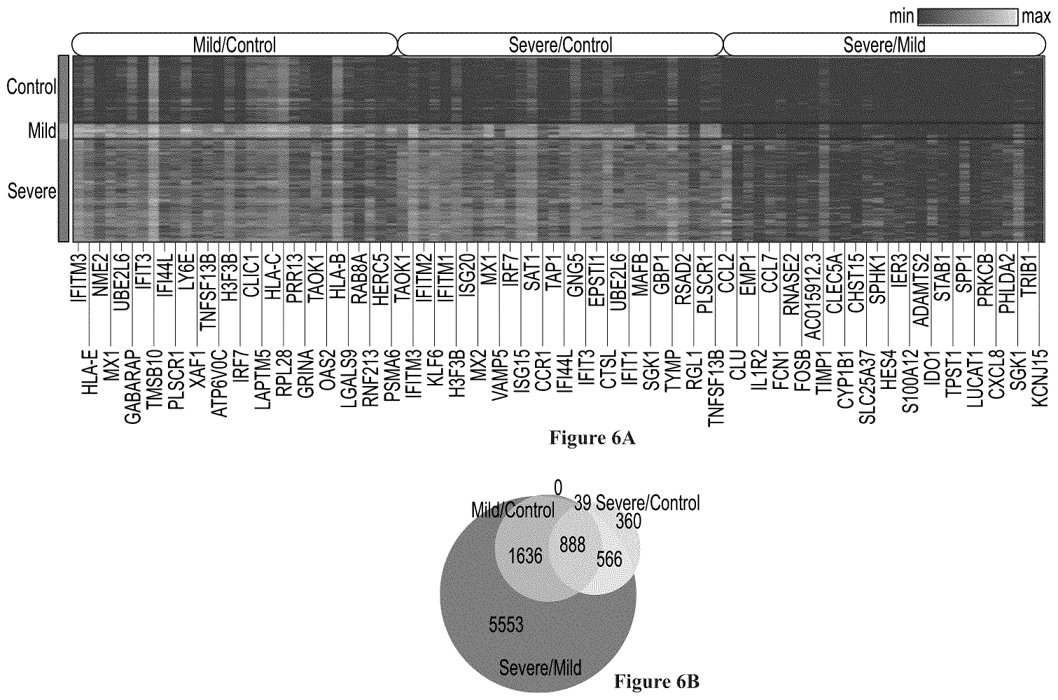

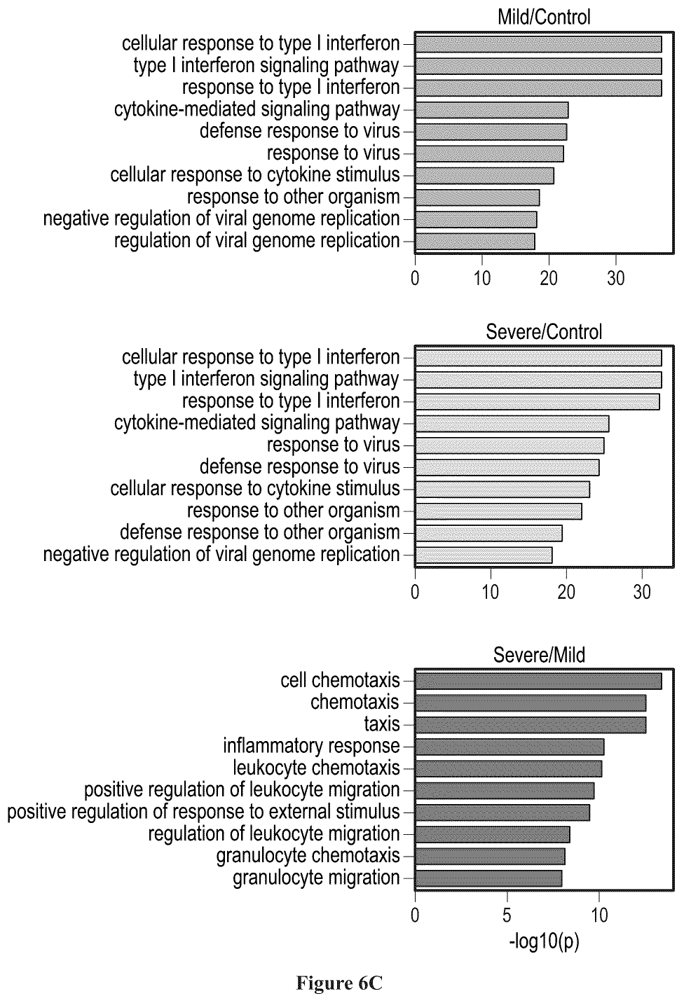

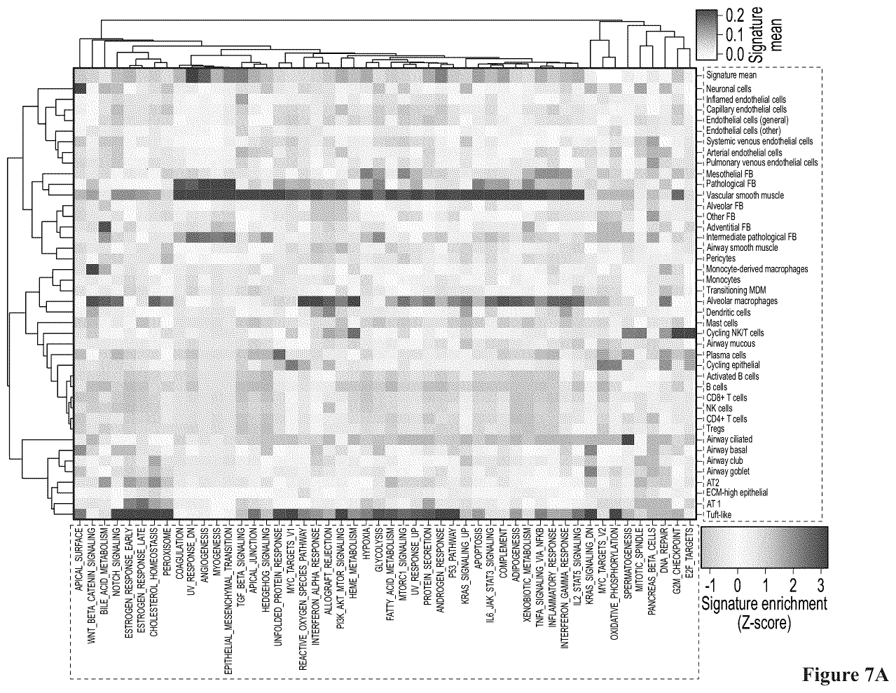

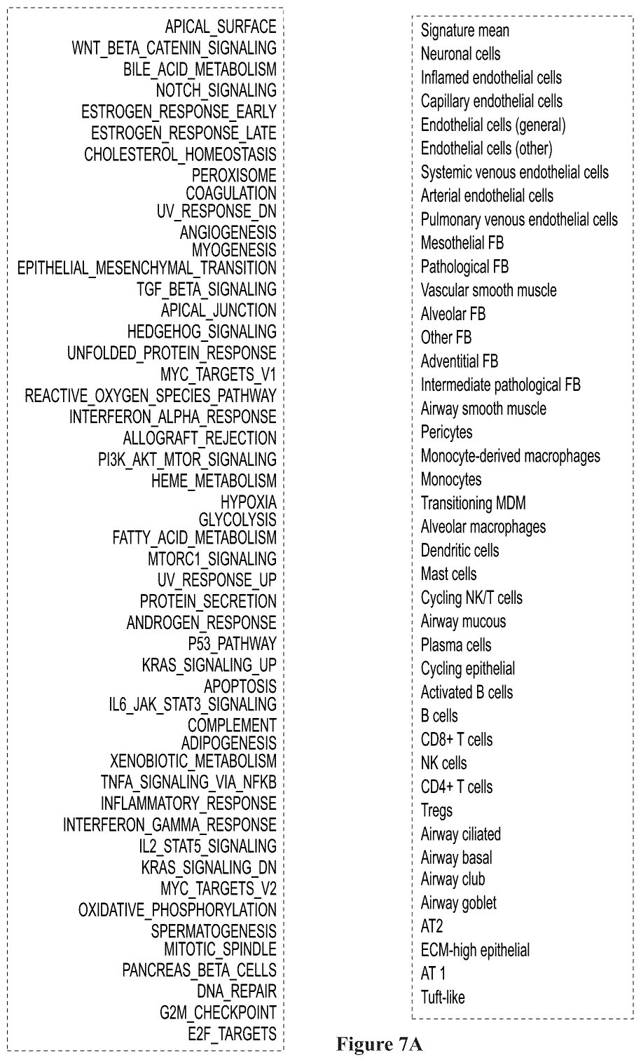

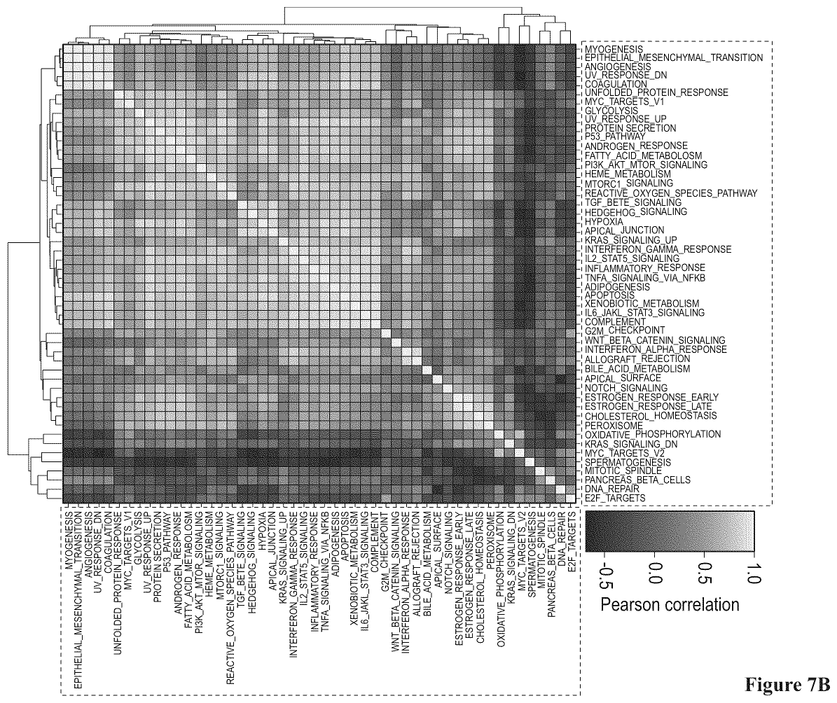

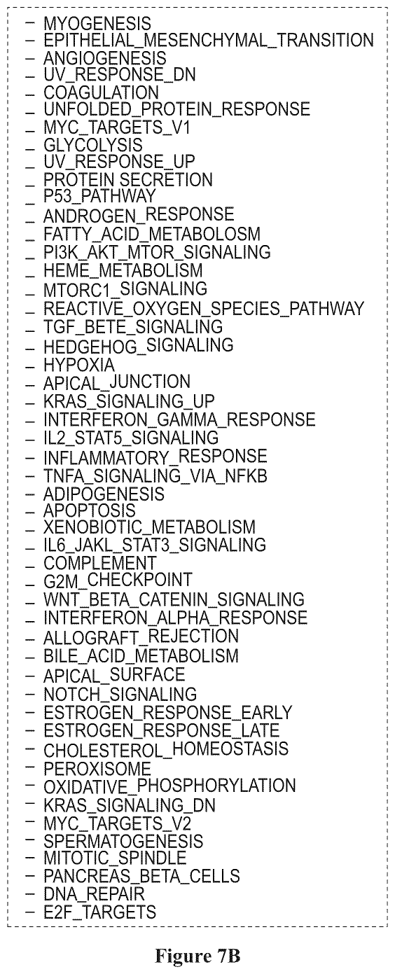

The relationship between IFN-I and inflammatory responses was analyzed in macrophages at the single-cell level and the two programs were found to be anti-correlated in controls, but correlated in COVID-19 patients, with macrophages from severe patients showing the highest correlation (FIG. 1F), as the response in mild patients was more dominated by the IFN-I response (FIG. 1F). This was confirmed by an orthogonal approach, using differential gene expression between the 3 populations of subjects and gene set enrichment. The enriched terms reinforced the conclusion that IFN-I is the main driver of the disease (FIGS. 6A-6A).

There was found to be a strong correlation between the IFN-I response and the presence of pro-fibrotic signals in macrophages. This observation led to the hypothesis that the heterogeneous information contained in the scRNA-seq data can be used to infer a pseudo-temporal continuum of the molecular response to SARS-CoV-2 infection in macrophages. Without being bound by theory, it is believed that patients at different stages of the disease could give an indication of the progression of the disease in SARS-CoV-2-infected patients. Using diffusion maps on the transcriptome data of macrophages, a joint representation was inferred which first dimension is associated with the dynamics of disease progression (FIG. 1G). Along this pseudotime axis, a high concentration of cells were observed from control patients and low IFN-I and inflammation was seen, followed by cells from mild patients with a peak of IFN-I signature, and lastly macrophages predominantly from severe patients were observed with high IFN-I and inflammation signatures (FIG. 1H). Similar observations have been made in influenza infection where macrophages both respond to interferon and are influenced by products of influenza infection and also a major producer of other cytokines. This response can be modified by virus components and host genetics resulting in altered macrophage responses that can lead to severe influenza infection (Kash, J. C. et al. Nature 443, 578-581, (2006); Kim, H. M. et al. J Virol 82, 4265-4274, (2008); Cardani, A., et al. PLoS Pathog 13, e1006140, (2017).

This unsupervised, dynamic view of macrophage response reinforces the idea of sequential stages of activation of the macrophages during COVID, associated with the early induction of IFN-I which primes macrophages for hyperinflammatory activation in a subset of patients which develop severe disease. To determine the source of the IFN-I, the dataset was analyzed and the infiltration of the BAL by pDCs in patients with mild disease was observed (FIG. 1I); while the number of pDCs was significantly reduced in patients with severe COVID-19, consistent with the observation by Liao, M. et al. Nat Med, 26, 842-844 (2020) (2020) (FIG. 1I).

These pDCs were found to express IFN-I-regulated genes (FIG. 1J) suggesting an activated phenotype, although transcripts of IFN-I were not detected, which could be due to the timing or to the fact that short/small transcripts, such as the IFN-I, are more frequently lost during scRNA-seq analysis. These data thus suggest that activated pDCs are transiently infiltrating the lung of SARS-CoV-2-infected individuals.

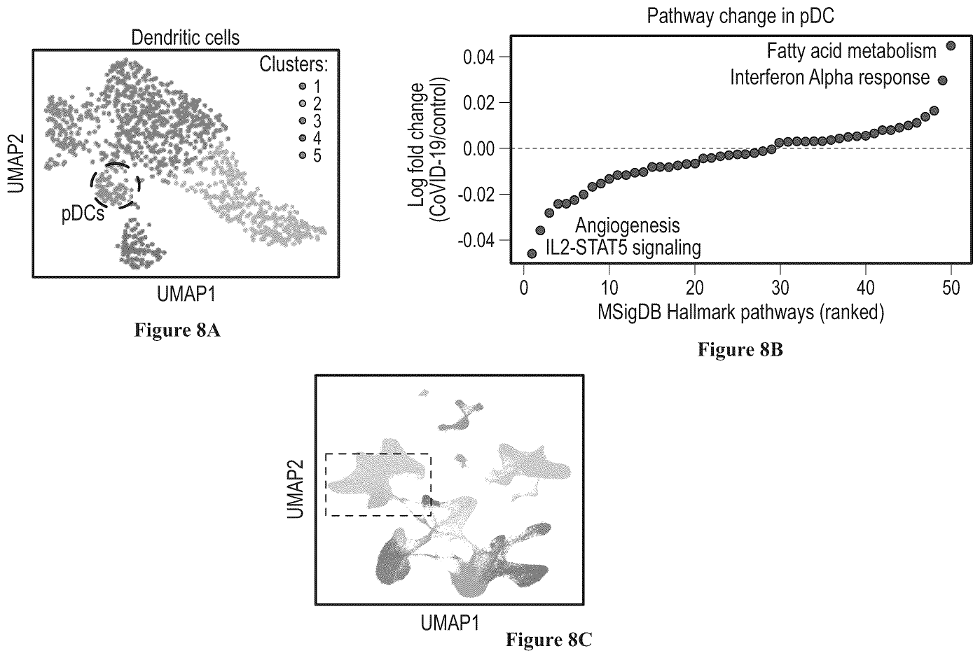

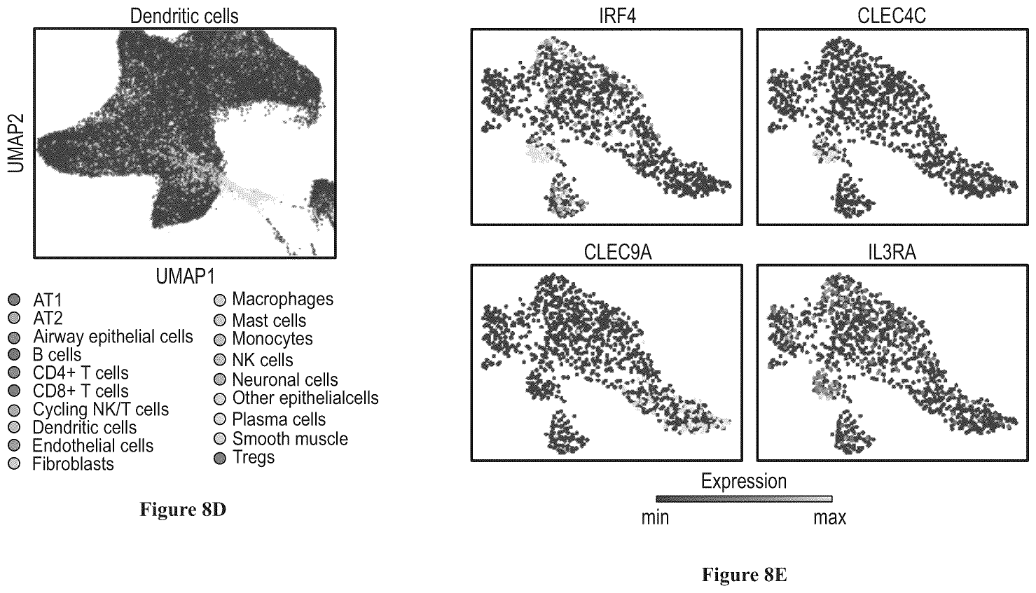

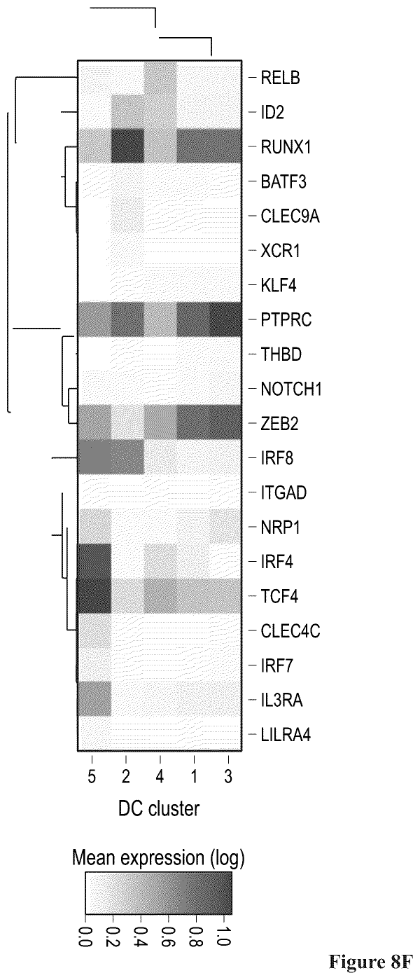

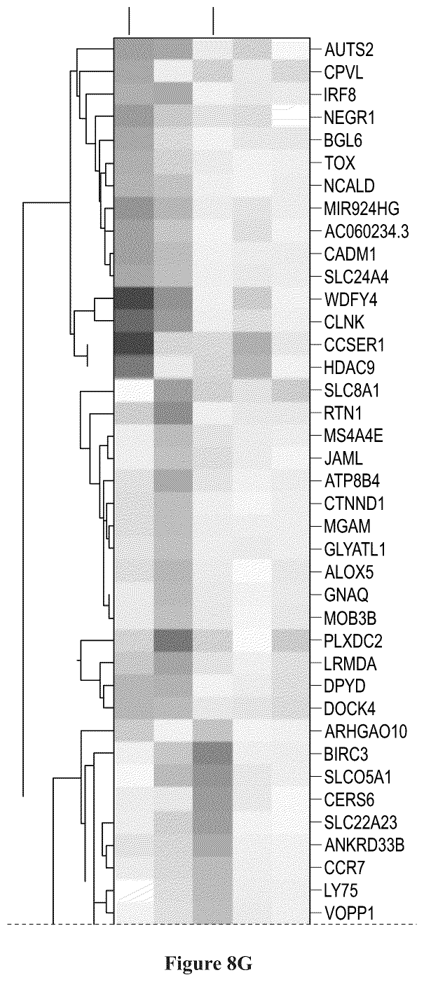

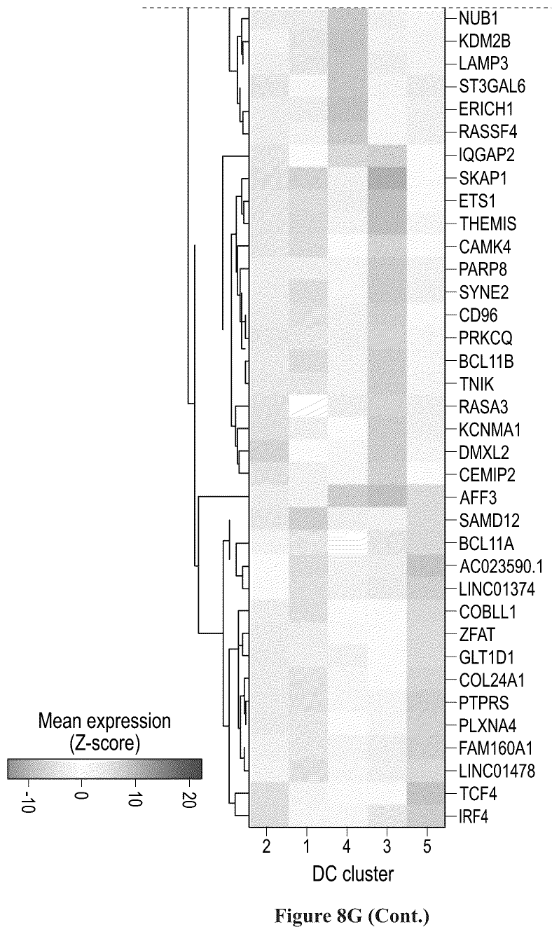

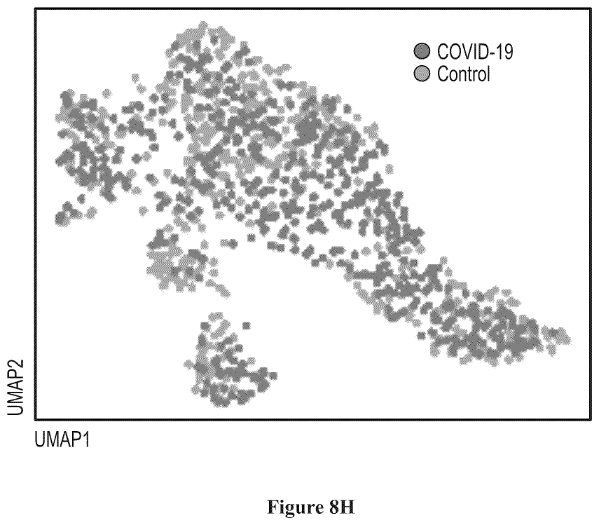

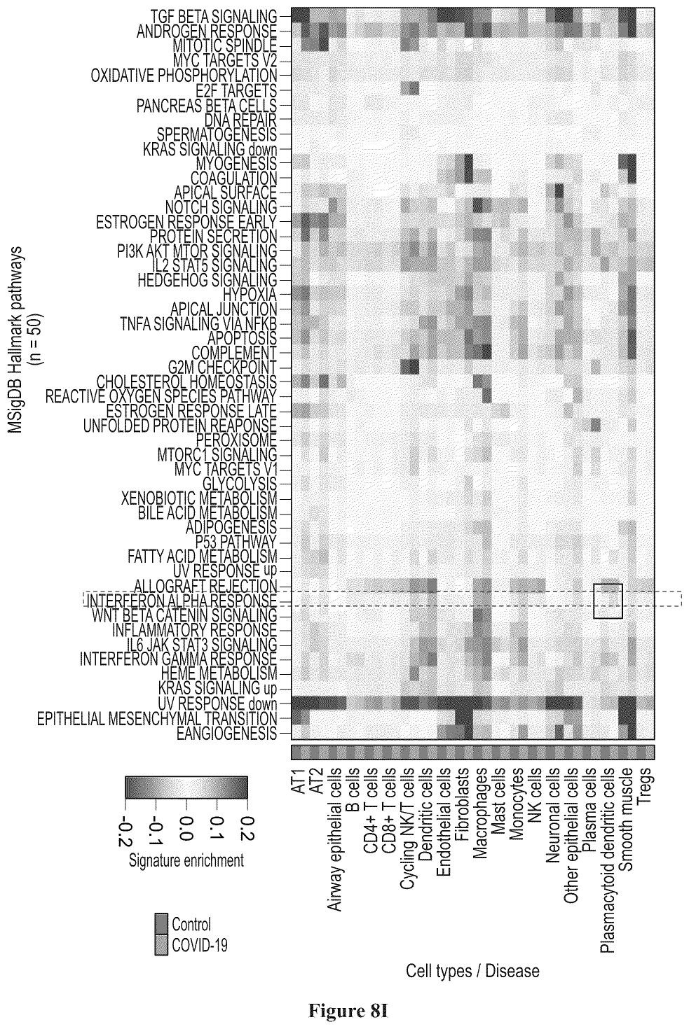

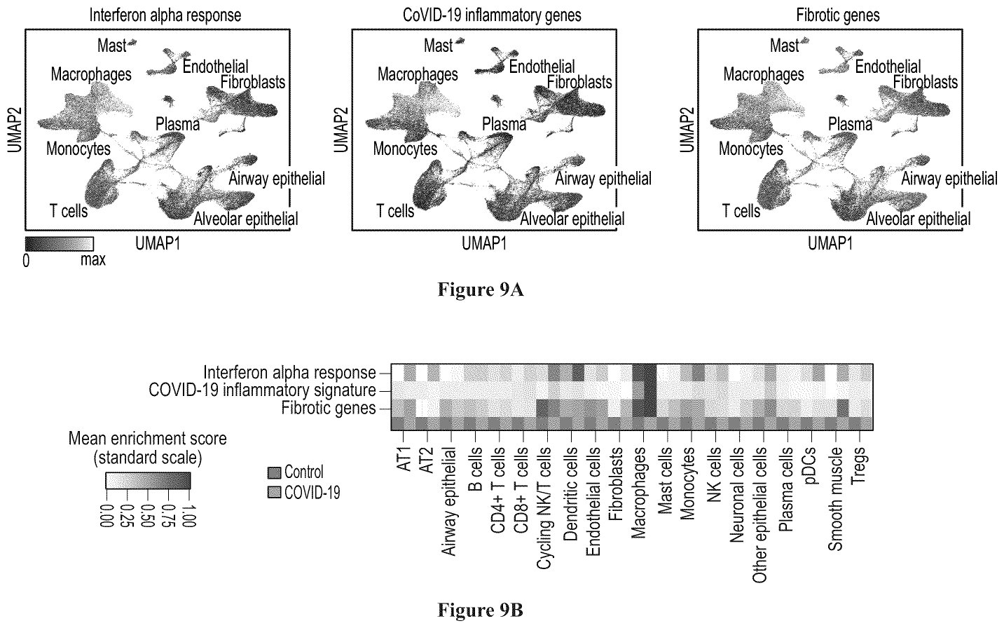

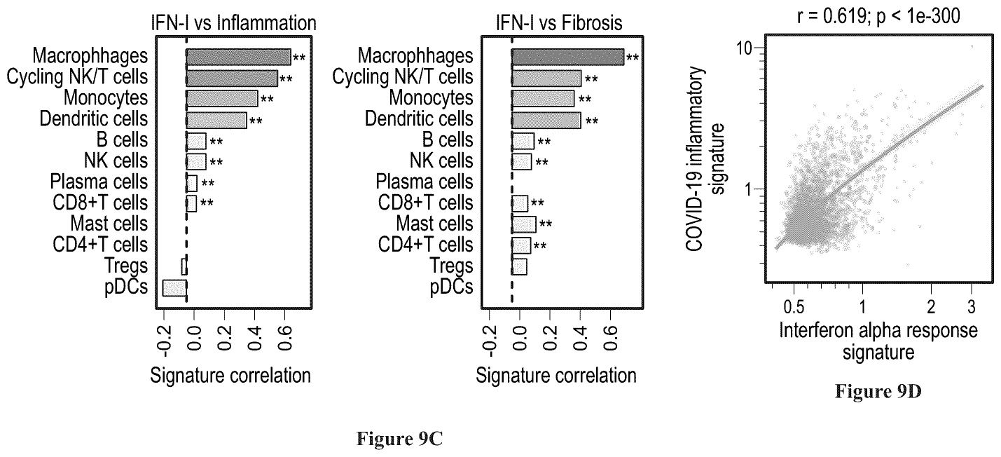

The cellular composition and activation status of lung cells in recently deceased patients following SARS-CoV-2 infection has been described (Melms, J. C. et al. Nature 595, 114-119, (2021); Rendeiro, A. F. et al. Nature 593, 564-569 (2021)). By conducting subcluster analysis in the dendritic cell subpopulation, pDCs were identified (FIGS. 8A, and 8C-8G) and by comparing to control lungs, the number of pDCs was found to be significantly reduced (FIG. 1K and FIG. 8H), although the remaining lung-infiltrating pDCs were found to express genes of the IFN pathways as a sign of their activation status in the lungs of the patients (FIGS. 8B and 8I). Next, the transcriptional profile of the lung cells was analyzed for the presence of genes associated with IFN-I response, inflammation or fibrosis (FIG. 9A and Table 1) and similar to the findings in the BAL data, the macrophage cluster was found to have a strong IFN-I response as well as higher expression of genes associated with inflammation and fibrosis (FIG. 9A). These signatures were present in other subsets as well but to a lesser degree than macrophages (FIG. 9A) and with higher expression in COVID-19 cells as compared to control (FIG. 9B and Table 2), suggesting disease-dependent induction of expression.

TABLE 2
Gene expression signatures in cell types

| A | B | T | dof | p-unc | BF10 |
|---|---|---|---|---|---|
| Ctrl | CD19 | −23.77519942 | 5457.48051 | 5.64E−119 | 2.99E+117 |
| Ctrl | CD19 | 5.650867459 | 8212.458103 | 1.65E−08 | 1.88E+05 |
| Ctrl | CD19 | −6.197934205 | 6835.440066 | 6.05E−10 | 4.72E+06 |
| Ctrl | CD19 | −24.09005112 | 4786.730449 | 3.98E−121 | 1.23E+121 |
| Ctrl | CD19 | 9.567014143 | 7712.966376 | 1.45E−21 | 1.27E+18 |
| Ctrl | CD19 | 9.300732079 | 7072.959289 | 1.82E−20 | 1.06E+17 |
| Ctrl | CD19 | −3.334664539 | 6115.736163 | 0.000859106 | 6.74 |
| Ctrl | CD19 | 1.135487103 | 7299.793992 | 0.256208562 | 0.05 |
| Ctrl | CD19 | 6.062764611 | 6556.805002 | 1.41E−09 | 2.33E+06 |
| Ctrl | CD19 | −0.402331779 | 1259.619065 | 0.687508133 | 0.058 |
| Ctrl | CD19 | 0.074441026 | 1391.164193 | 0.940670167 | 0.054 |
| Ctrl | CD19 | 2.399979921 | 1618.711904 | 0.016508334 | 0.924 |
| Ctrl | CD19 | −9.054832742 | 6328.586767 | 1.79E−19 | 1.27E+16 |
| Ctrl | CD19 | −1.264615154 | 6957.396433 | 0.206051677 | 0.06 |
| Ctrl | CD19 | −5.272181453 | 5968.467566 | 1.40E−07 | 2.78E+04 |
| Ctrl | CD19 | −12.73931803 | 1671.222409 | 1.52E−35 | 8.80E+32 |
| Ctrl | CD19 | −5.347799355 | 1630.219774 | 1.02E−07 | 6.36E+04 |
| Ctrl | CD19 | −1.619520898 | 1426.939253 | 0.105556203 | 0.166 |
| Ctrl | CD19 | −8.487196502 | 1317.891488 | 5.62E−17 | 1.11E+14 |
| Ctrl | CD19 | −6.848109673 | 1654.990187 | 1.05E−11 | 5.41E+08 |
| Ctrl | CD19 | 3.303183838 | 1037.240201 | 0.000988594 | 11.271 |
| Ctrl | CD19 | −7.492556322 | 805.6779812 | 1.78E−13 | 3.70E+10 |
| Ctrl | CD19 | −1.038002758 | 916.1970878 | 0.299542634 | 0.12 |
| Ctrl | CD19 | −0.89777625 | 1011.573202 | 0.369518472 | 0.105 |
| Ctrl | CD19 | −7.366265612 | 5383.88515 | 2.02E−13 | 1.55E+10 |
| Ctrl | CD19 | 9.921575946 | 5138.530554 | 5.39E−23 | 4.20E+19 |
| Ctrl | CD19 | 2.693722027 | 4858.679371 | 0.007090182 | 1.172 |
| Ctrl | CD19 | −35.51192444 | 19781.17117 | 7.87E−268 | 1.44E+264 |
| Ctrl | CD19 | 11.49243403 | 17078.88923 | 1.86E−30 | 6.78E+26 |
| Ctrl | CD19 | −26.77346248 | 15456.81329 | 2.14E−154 | 1.76E+151 |
| Ctrl | CD19 | −3.275055859 | 5448.475359 | 0.001063013 | 4.116 |
| Ctrl | CD19 | −24.3837368 | 10983.02929 | 6.17E−128 | 6.52E+125 |
| Ctrl | CD19 | −2.865137943 | 5771.547107 | 0.004183334 | 1.171 |
| Ctrl | CD19 | −4.68588865 | 1361.6746 | 3.07E−06 | 2934.872 |
| Ctrl | CD19 | 0.608459816 | 1103.720549 | 0.543007743 | 0.073 |
| Ctrl | CD19 | −4.450826096 | 1208.734746 | 9.34E−06 | 1028.878 |
| Ctrl | CD19 | −11.28640494 | 2025.866542 | 1.08E−28 | 4.92E+25 |
| Ctrl | CD19 | −7.368045464 | 2367.127733 | 2.38E−13 | 2.04E+10 |
| Ctrl | CD19 | 1.229438238 | 1115.752504 | 0.219166737 | 0.103 |
| Ctrl | CD19 | −8.337697311 | 1787.766215 | 1.49E−16 | 2.91E+13 |
| Ctrl | CD19 | −5.178950483 | 1972.409135 | 2.46E−07 | 2.86E+04 |
| Ctrl | CD19 | 1.317391341 | 1424.149092 | 0.187919385 | 0.121 |
| Ctrl | CD19 | −7.806089352 | 1076.064269 | 1.39E−14 | 5.20E+11 |
| Ctrl | CD19 | −0.332650215 | 687.8367278 | 0.739499689 | 0.071 |
| Ctrl | CD19 | −4.157195961 | 706.9365489 | 3.62E−05 | 329.204 |
| Ctrl | CD19 | −4.923647956 | 1774.720449 | 9.28E−07 | 8819.59 |
| Ctrl | CD19 | −4.393562918 | 1771.953508 | 1.18E−05 | 786.095 |
| Ctrl | CD19 | −2.855369528 | 1783.977919 | 0.004348398 | 3.275 |
| Ctrl | CD19 | 2.425261946 | 1649.785642 | 0.015404057 | 0.772 |
| Ctrl | CD19 | 14.18368303 | 2588.42444 | 4.94E−44 | 2.12E+41 |
| Ctrl | CD19 | −0.272897738 | 1527.577697 | 0.784968712 | 0.043 |
| Ctrl | CD19 | −2.014543344 | 23.45079842 | 0.05555904 | 1.457 |
| Ctrl | CD19 | −0.786112233 | 62.25043177 | 0.434783985 | 0.361 |
| Ctrl | CD19 | −0.795651695 | 33.47316003 | 0.431840912 | 0.364 |
| Ctrl | CD19 | −9.228374024 | 1389.025423 | 9.92E−20 | 3.89E+16 |
| Ctrl | CD19 | −3.263091652 | 1290.651322 | 0.001130888 | 11.676 |
| Ctrl | CD19 | −8.285577105 | 1363.261453 | 2.78E−16 | 1.63E+13 |
| Ctrl | CD19 | −5.776026074 | 542.2755824 | 1.29E−08 | 7.65E+05 |
| Ctrl | CD19 | −0.034199954 | 497.378615 | 0.972731423 | 0.093 |
| Ctrl | CD19 | −1.963847966 | 505.0713922 | 0.0500958 | 0.606 |

| hedges | median_A | median_B | p-cor | Cell type |
|---|---|---|---|---|
| −0.496786989 | −0.003810976 | 0.016588953 | 3.16E−117 | AT1 |
| 0.116507404 | −0.021604938 | −0.024691358 | 5.77E−07 | AT1 |
| −0.128618519 | 0.045380117 | 0.044678363 | 2.30E−08 | AT1 |
| −0.563698343 | −0.011791607 | 0.009041726 | 2.27E−119 | AT2 |
| 0.194770727 | −0.026234568 | −0.029320988 | 7.25E−20 | AT2 |
| −0.193825341 | −0.018479532 | −0.005146199 | 8.94E−19 | AT2 |
| −0.079014849 | −0.008234696 | −0.005903276 | 0.022336763 | Airway |
| | | | | epithelial |
| | | | | cells |
| 0.026527182 | −0.02637486 | −0.025533109 | 1 | Airway |
| | | | | epithelial |
| | | | | cells |
| 0.143106449 | 0.04748538 | 0.017894737 | 5.23E−08 | Airway |
| | | | | epithelial |
| | | | | cells |
| −0.02026795 | 0.023388929 | 0.021954209 | 1 | B cells |
| 0.003659626 | −0.024691358 | −0.021604938 | 1 | B cells |
| 0.113264045 | 0.045263158 | 0.028304094 | 0.297150008 | B cells |
| −0.20783709 | 0.015841703 | 0.020519488 | 8.42E−18 | CD4+ T |
| | | | | cells |
| −0.027938899 | −0.020061728 | −0.021604938 | 1 | CD4+ T |
| | | | | cells |
| −0.123568058 | 0.019298246 | 0.02374269 | 4.61E−06 | CD4+ T |
| | | | | cells |
| −0.442773181 | 0.010102822 | 0.022641679 | 8.20E−34 | CD8+ T |
| | | | | cells |
| −0.188198995 | −0.016975309 | −0.016975309 | 3.46E−06 | CD8+ T |
| | | | | cells |
| −0.061325969 | 0.017076023 | 0.018596491 | 1 | CD8+ T |
| | | | | cells |
| −0.332235574 | 0.009161286 | 0.030936155 | 2.58E−15 | Cycling |
| | | | | NK/T cells |
| −0.242081869 | −0.041666667 | −0.024691358 | 4.10E−10 | Cycling |
| | | | | NK/T cells |
| 0.148398531 | 0.097894737 | 0.080116959 | 0.024714838 | Cycling |
| | | | | NK/T cells |
| −0.463826798 | 0.03130978 | 0.056663977 | 7.47E−12 | Dendritic |
| | | | | cells |
| −0.064540493 | −0.026234568 | −0.023218294 | 1 | Dendritic |
| | | | | cells |
| −0.056214267 | 0.049707602 | 0.060935673 | 1 | Dendritic |
| | | | | cells |
| −0.186474223 | 0.011911167 | 0.015871593 | 8.28E−12 | Endothelial |
| | | | | cells |
| 0.262664379 | −0.029320988 | −0.035493827 | 2.75E−21 | Endothelial |
| | | | | cells |
| 0.073023823 | 0.074152047 | 0.051929825 | 0.14180365 | Endothelial |
| | | | | cells |
| −0.376218493 | −0.018217958 | −0.001748565 | 4.72E−266 | Fibroblasts |
| 0.129769857 | −0.032407407 | −0.038580247 | 9.88E−29 | Fibroblasts |
| −0.314143964 | −0.000701754 | 0.030409357 | 1.26E−152 | Fibroblasts |
| −0.057274798 | 0.039230631 | 0.052606408 | 0.025512301 | Macrophages |
| −0.267975132 | 0.001402918 | 0.033810325 | 3.58E−126 | Macrophages |
| −0.046952885 | 0.07497076 | 0.097192982 | 0.092033356 | Macrophages |
| −0.24076705 | 5.98E−05 | 0.005425036 | 9.20E−05 | Mast cells |
| 0.033576509 | −0.023148148 | −0.021604938 | 1 | Mast cells |
| −0.239002757 | 0.02374269 | 0.051929825 | 0.00027096 | Mast cells |
| −0.330439204 | 0.027879902 | 0.042413917 | 5.61E−27 | Monocytes |
| −0.204091584 | −0.021604938 | −0.018518519 | 9.52E−12 | Monocytes |
| 0.048113252 | 0.069707602 | 0.058596491 | 1 | Monocytes |
| −0.353303661 | 0.018711143 | 0.030936155 | 6.71E−15 | NK cells |
| −0.210271129 | −0.018518519 | −0.015432099 | 7.87E−06 | NK cells |
| 0.060731833 | 0.025964912 | 0.02374269 | 1 | NK cells |
| −0.290964262 | −0.008174916 | 5.98E−05 | 5.99E−13 | Neuronal cells |
| −0.015006964 | −0.024691358 | −0.026234568 | 1 | Neuronal cells |
| −0.184846013 | 0.00374269 | 0.013391813 | 0.000976647 | Neuronal cells |
| −0.192837521 | 0.005858441 | 0.019458393 | 2.88E−05 | Other |
| | | | | epithelial |
| | | | | cells |
| −0.165010171 | −0.027777778 | 0.016975309 | 0.000330705 | Other |
| | | | | epithelial |
| | | | | cells |
| −0.110520682 | 0.046081871 | 0.034853801 | 0.092033356 | Other |
| | | | | epithelial |
| | | | | cells |
| 0.071111897 | 0.007233381 | 0.005425036 | 0.292677089 | Plasma cells |
| 0.335464375 | −0.023148148 | −0.032407407 | 2.72E−42 | Plasma cells |
| −0.008422475 | 0.025964912 | 0.025263158 | 1 | Plasma cells |
| −0.619197203 | 0.012972262 | 0.046030607 | 0.888944641 | Plasmacytoid |
| | | | | dendritic cells |
| −0.150572051 | −0.033950617 | −0.032407407 | 1 | Plasmacytoid |
| | | | | dendritic cells |
| −0.198588568 | 0.017076023 | 0.021520468 | 1 | Plasmacytoid |
| | | | | dendritic cells |
| −0.425653789 | −0.016095768 | 0.000216703 | 4.76E−18 | Smooth muscle |
| −0.14618803 | −0.044051627 | −0.033950617 | 0.026010427 | Smooth muscle |
| −0.378649557 | −0.005146199 | 0.025964912 | 1.22E−14 | Smooth muscle |
| −0.440031013 | 0.014033357 | 0.023015304 | 4.64E−07 | Tregs |
| −0.002694361 | −0.021604938 | −0.021604938 | 1 | Tregs |
| −0.153819873 | 0.015614035 | 0.021520468 | 0.851628594 | Tregs |

For all measures, Paired criteria is FALSE and Parametric criteria is TRUE; Alternative is two-sided. Ctrl : Contrl; CD19: COVID-19

A similar positive correlation was also observed at the pathway level between the IFN pathway and both the inflammation and fibrotic pathways (FIG. 9C). These correlations were present in more than one cellular subset, including other myeloid cells such as DCs or monocytes, but were the highest in the macrophage cluster (FIG. 9C). Next, macrophages at the single cell level were investigated and a robust and highly significant correlation between the presence of IFN-induced signaling and the expression of proinflammatory cytokines was observed (FIG. 1L and FIG. 9D). Subsets of macrophages were investigated. While both resident alveolar macrophages, and infiltrating monocyte-derived macrophages were found to express a COVID-19-related inflammatory response, the IFN-I response and fibrotic response were mostly restricted to the monocyte-derived macrophage subset (FIGS. 1M-1N).

These data would indicate that during the course of the disease, the intense priming of macrophages by pDC-derived IFN-I, which likely occurs early in the disease pathogenesis, can prime macrophages and in particular monocyte-derived macrophages.

### Example 2. pDCs Sense SARS-CoV-2 Via TLR7 and are the Dominant IFN-I Producing Blood Cells in Response to the Virus

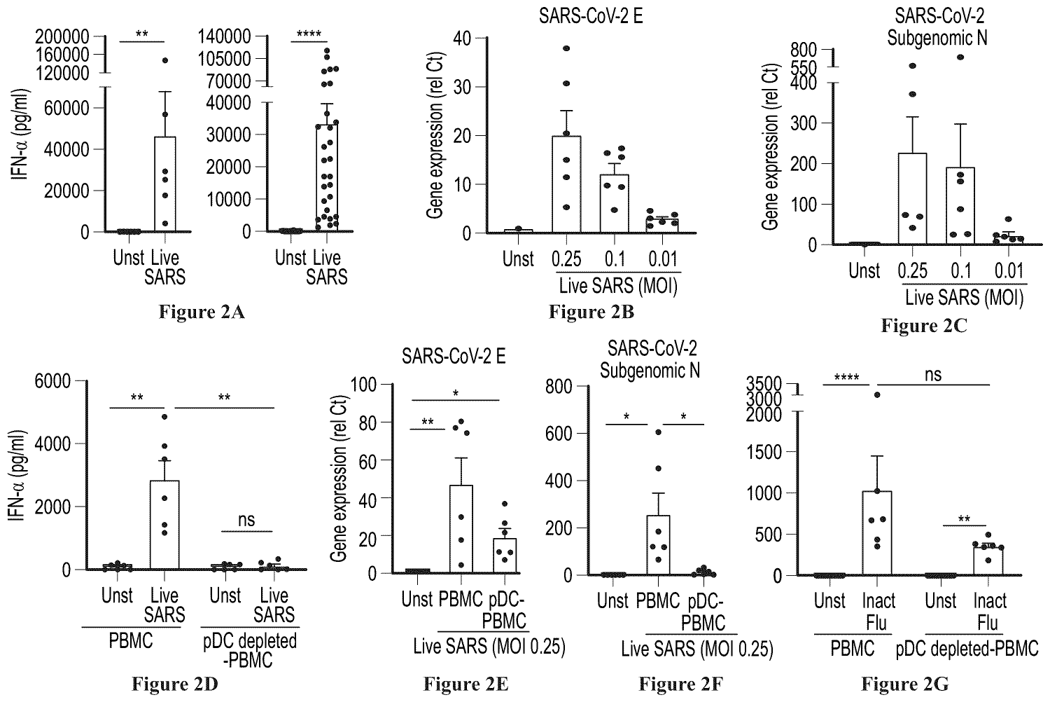

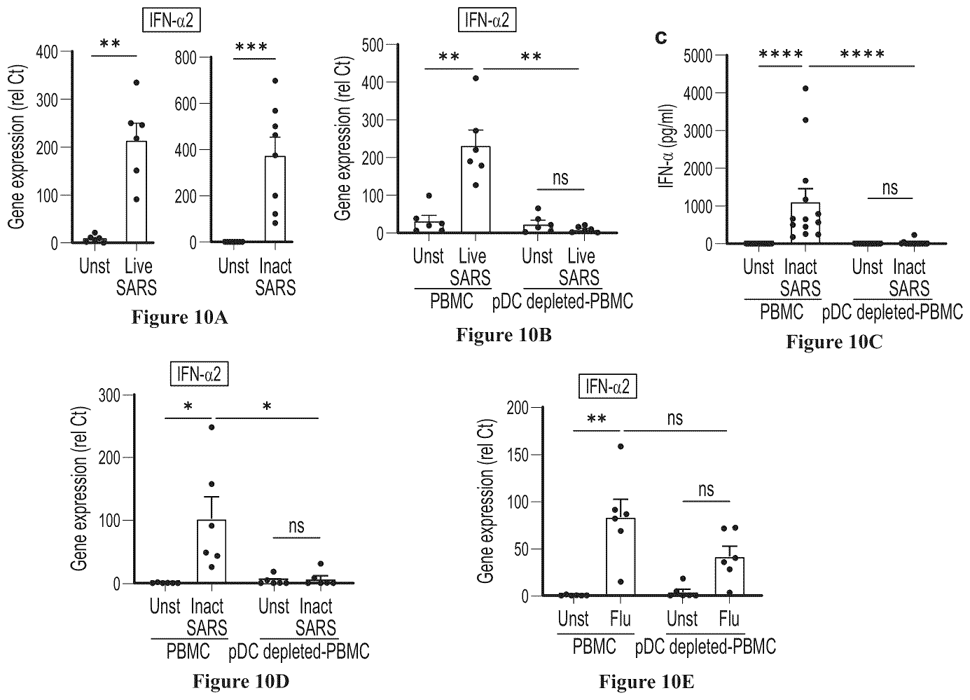

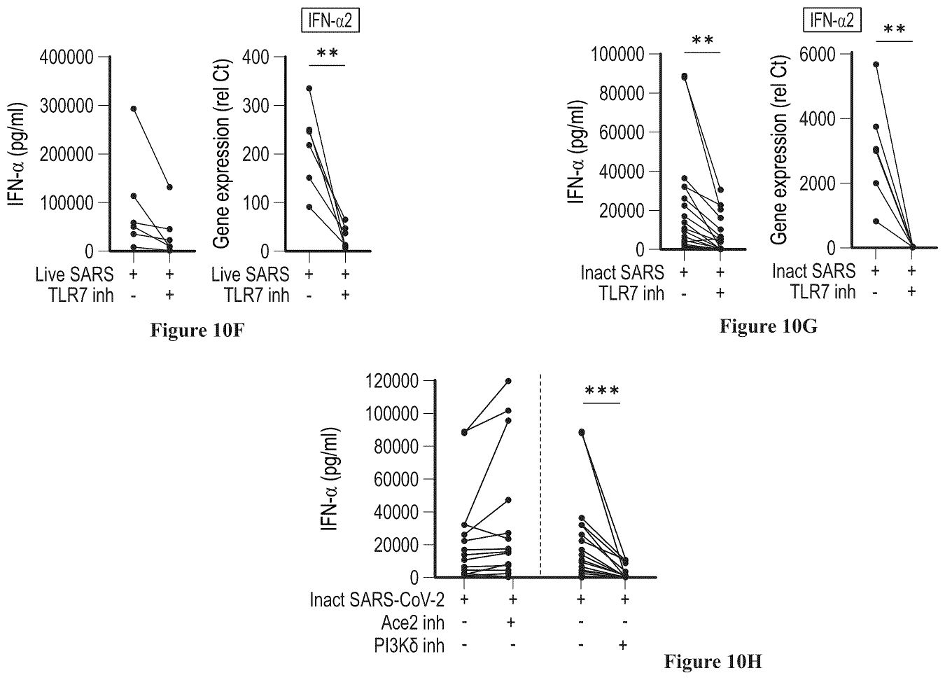

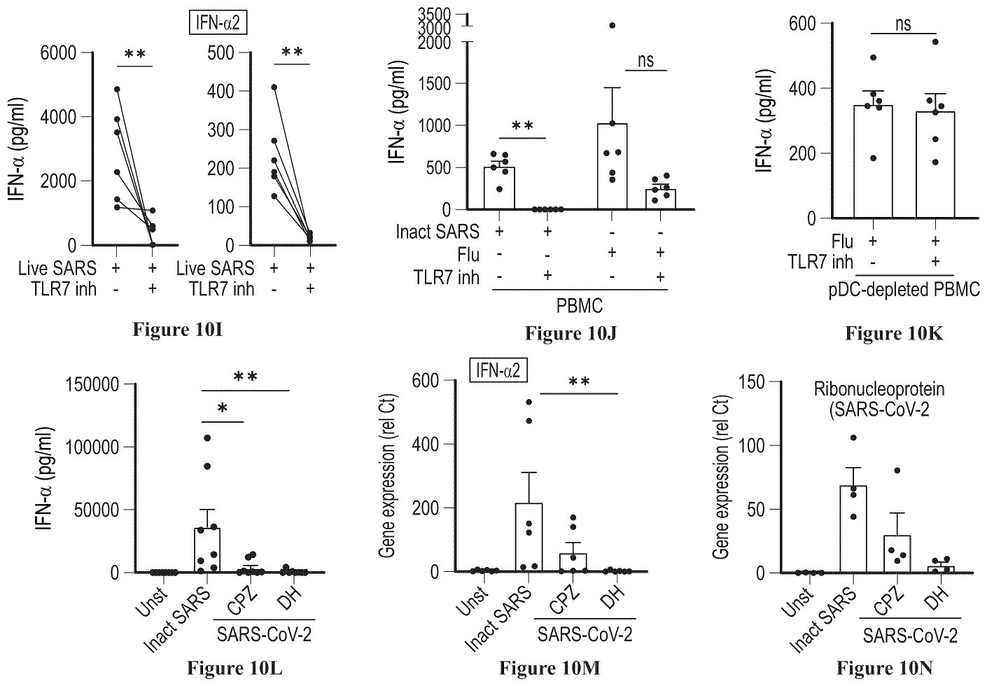

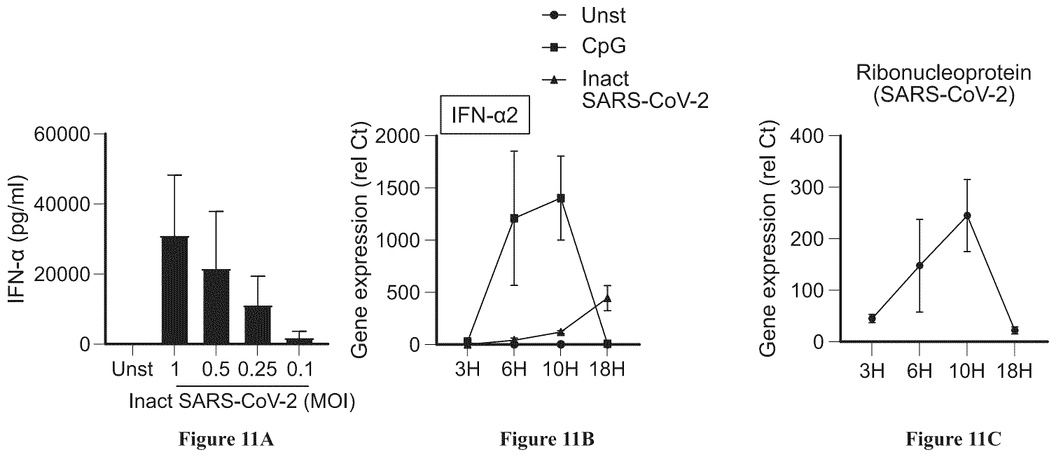

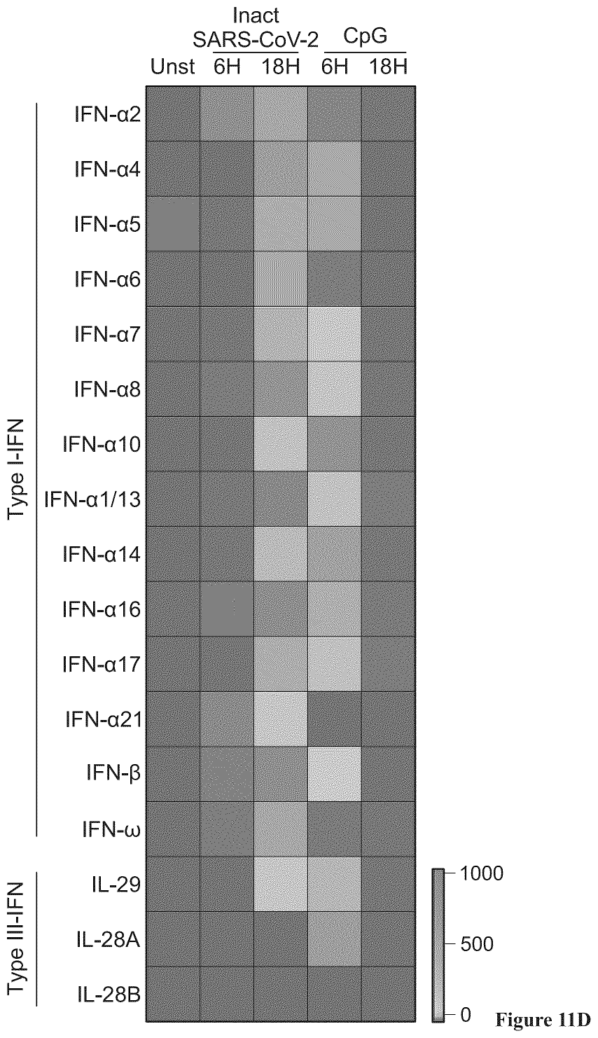

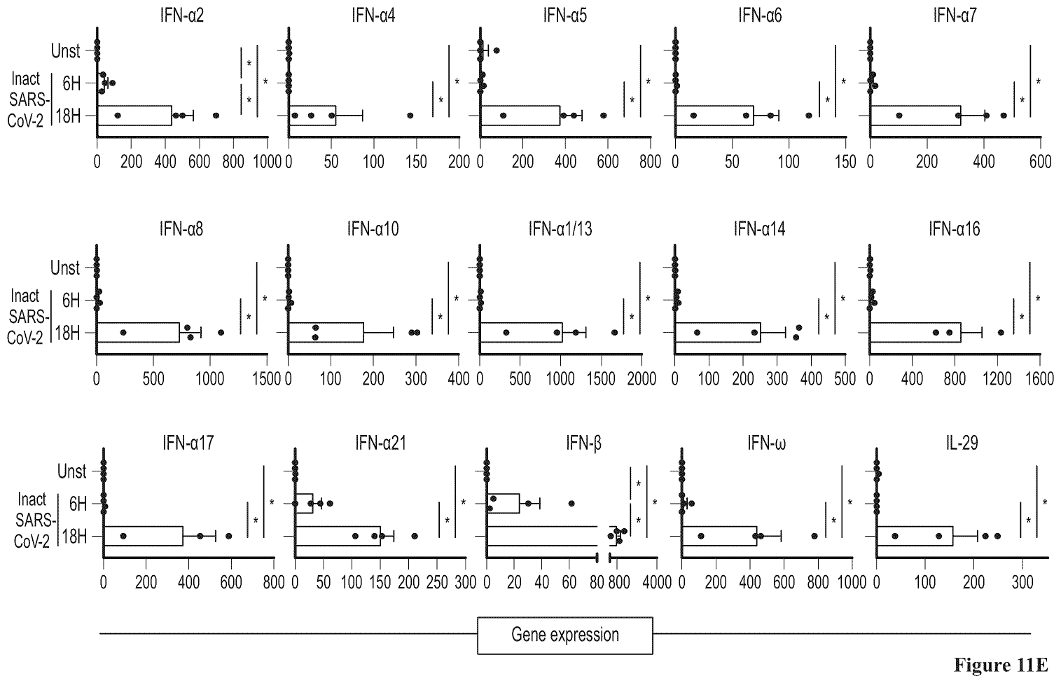

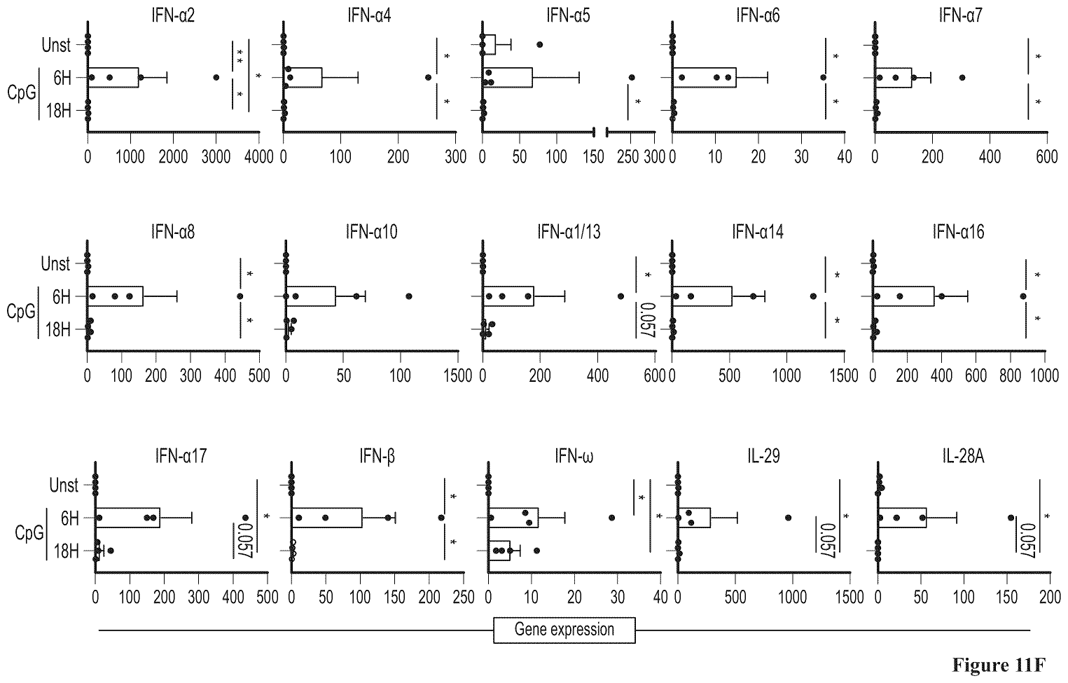

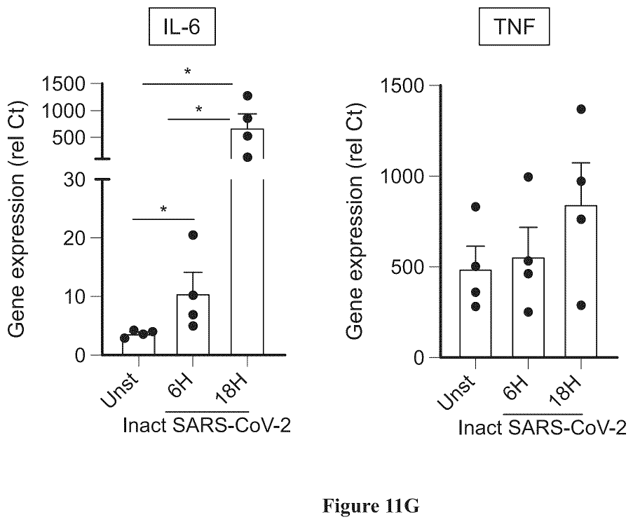

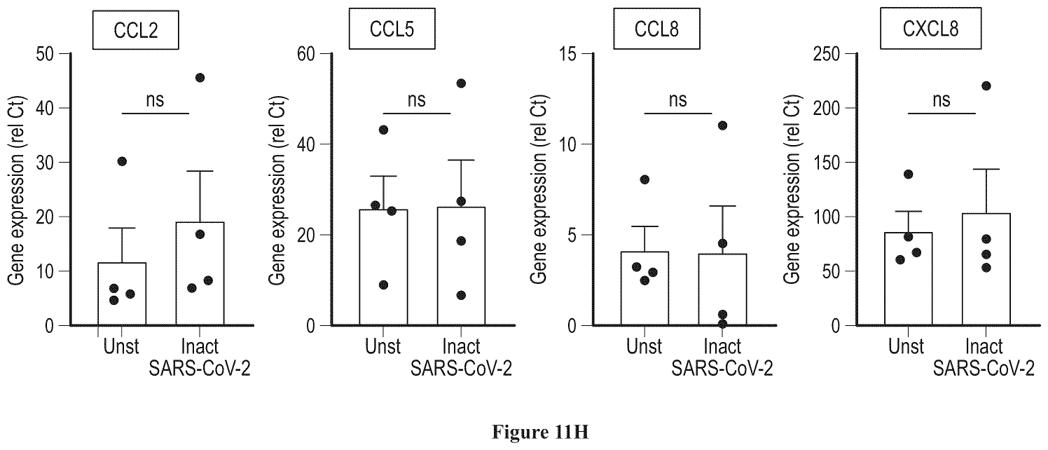

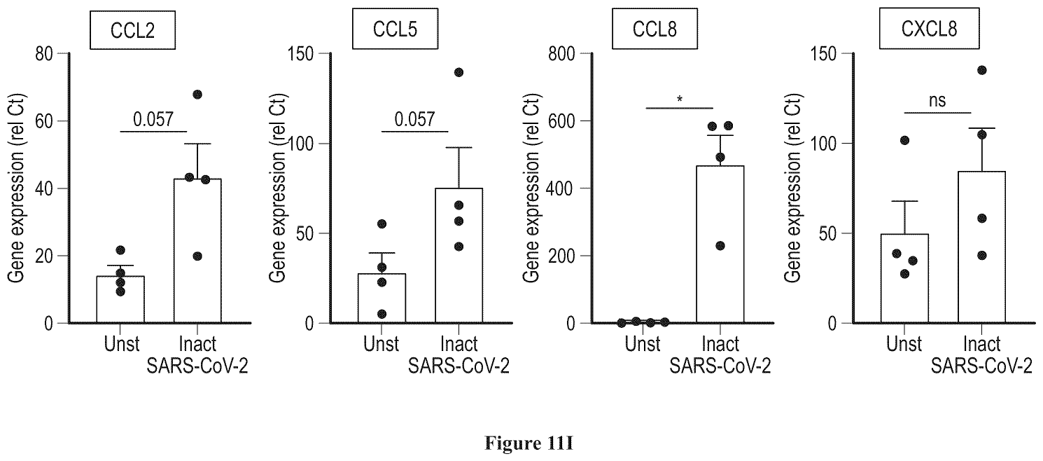

Although IFN-180 I is critical for the clinical response to SARS-CoV-2 infection, the cellular source of IFN-I is not well defined. Purified pDCs were found to have a robust IFN-α response to both live, but also UV-inactivated SARS-CoV-2 (FIG. 2A and FIGS. 10A and 11A), consistent with earlier findings (Severa, M. et al. PLoS Pathog 17, e1009878, (2021); Onodi, F. et al. J Exp Med 218, (2021); Asano, T. et al. Science immunology 6, (2021), and it was found that the virus was able to efficiently infect the pDCs (FIG. 2B) and replicate in pDCs (FIG. 2C). The relative contribution of pDCs to the overall IFN-I response to SARS-CoV-2 by PBMC was then evaluated. Although total PBMCs could produce significant amounts of IFN-α when incubated with either live or inactivated virus, the production of IFN-α in response to live or inactivated SARS-CoV-2 by PBMC depleted of the pDCs (pDC depleted PBMC), was negligible (FIG. 2D and FIG. 10B-10D). Consistent with these data, the amount of replicating SARS-CoV-2 in pDC-depleted PBMC was also negligible (FIGS. 2E-2F). This is in contrast to similar conditions but using influenza virus (Flu) where the depletion of pDCs only partially reduced the overall IFN-α response (FIG. 2G and FIG. 10E). These data demonstrate that, in contrast to Flu where pDCs but also other cells can produce IFN-α, pDCs are the dominant producers of IFN-α in the blood in response to SARS-CoV-2. Inactivated SARS-CoV-2-induced IFN-α showed a progressive curve, which was higher at 18 h than at the typical 6-10 h time points, as for the TLR9 agonist CpG (FIG. 11B). However, the nature of the response by pDCs to SARS-CoV-2 was similar to what is known for TLR7/9 signaling, with the induction of all subtypes of IFN-I (FIG. 11D-11F), of IL-6 (FIG. 11G) and of a series of chemokines which likely contributes to the migration of immune cells into the lungs of patients (FIGS. 11H-11I). The difference in the kinetics of the response by pDCs between inactivated SARS-CoV-2 and CpG is likely due to delay in entry of the virus (FIG. 11C) as compared to what is known for CpG (Guiducci, C. et al., J Exp Med 203, 1999-2008 (2006). As SARS-CoV-2 is a single stranded RNA virus, the mechanism of activation of the pDCs by nucleic acid-sensing pathways was explored. First, the contribution of ACE2 was excluded, as these cells have little to no expression of ACE2 (not shown and Onodi, F. et al. J Exp Med 218, (2021) and adding an ACE2 inhibitor had no effect on the IFN-α production by SARS pDCs (FIG. 10H). In contrast, blocking TLR740 or PI3Kδ, which is key to TLR7-induced IFN-α in pDCs (Guiducci, C. et al. J Exp Med 205, 315-322 (2008) led to the drastic inhibition of the IFN-α response (FIGS. 10F-H). Consistent with this observation that pDCs are the dominant producer of IFN-α in the blood, a complete inhibition of IFN-α production by the TLR7 inhibitor was observed in PBMC as well (FIG. 10I). Although it is documented that pDCs sense Flu via TLR740, this is different when using Flu, as the inhibition of TLR7 in PBMC only partially reduces the IFN-α response to influenza (Flu) (FIG. 10J) and had no effect when using pDC-depleted PBMCs (FIG. 10K). This finding is consistent with the observation that pDCs from TLR7-deficient patients have a poor response to SARS-CoV-28, although the overall response to SARS-CoV-2 by pDCs isolated from the TLR7-deficient patients was not complete. This may be due to redundancy developed by these cells with germline mutations of TLR7 to virus sensing, as was observed with IRAK4-deficient patients (see Yang et al. Immunity 2005). It is also likely that other cell types which bears TLR7 may be involved in the response SARS-CoV-2, in particular in the tissue environment. Nucleic acid-sensing TLRs are located in endosomal compartments (Barrat, F. J. & Su, L. J Exp Med 216, 1974-1985, doi:10.1084/jem.20181359 (2019); Reizis, B. Immunity 50, 37-50, doi:10.1016/j.immuni.2018.12.027 (2019); Guiducci, C. et al. J Exp Med 203, 1999-2008 (2006) and it was observed that the entry of SARS-CoV-2 and the subsequent induction of IFN-α requires clathrin-mediated endocytosis (FIG. 10L-10N). These data thus demonstrate that SARS-CoV-2 can enter pDCs using clathrin-mediated entry and is sensed by TLR7 which signals to trigger IFN-I production.

It has been described that less than 10% of lung macrophages are infected with SARS-CoV-2 (Rendeiro, A. F. et al. Nature 593, 564-569, (2021). However, it was recently reported that macrophages produce some IFN-I due to the activation of the cGAS-STING pathway (Lore, K., et al. J. Exp Med. 201, 2023-2033 (2005)), as the deletion of STING in a mouse model of SARS infection partially reduced IFN-I and ISG expression. However, the authors did not observe that macrophages can be directly stimulated by SARS-CoV-2.

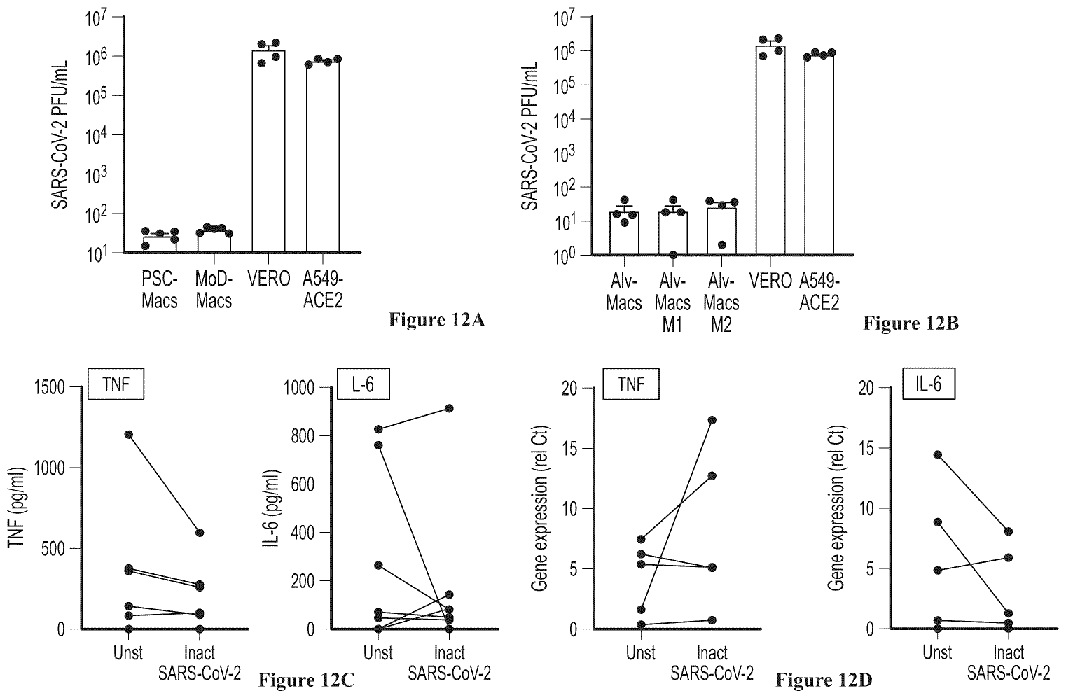

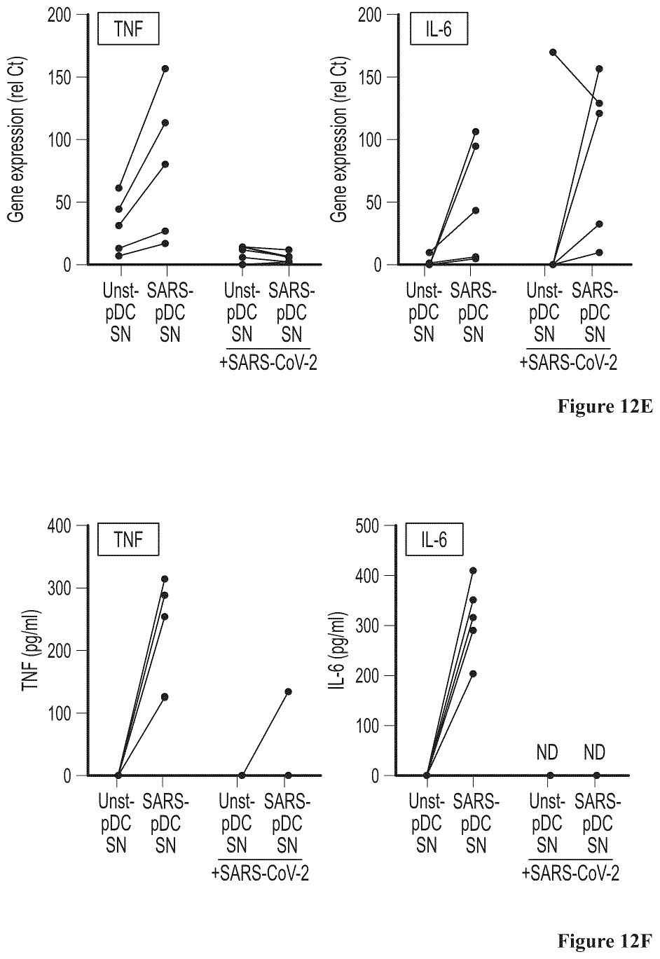

Hence, it was observed that neither CD14+ monocytes, pluripotent stem cells (PSCs) derived macrophages, monocyte derived macrophages or primary alveolar macrophages isolated from human lungs can directly be infected nor stimulated by live SARS-CoV-2 (FIGS. 12A-12B) and inactivated SARS-CoV-2 induced little to no TNF and IL-6 in macrophages (FIGS. 12C-12D).

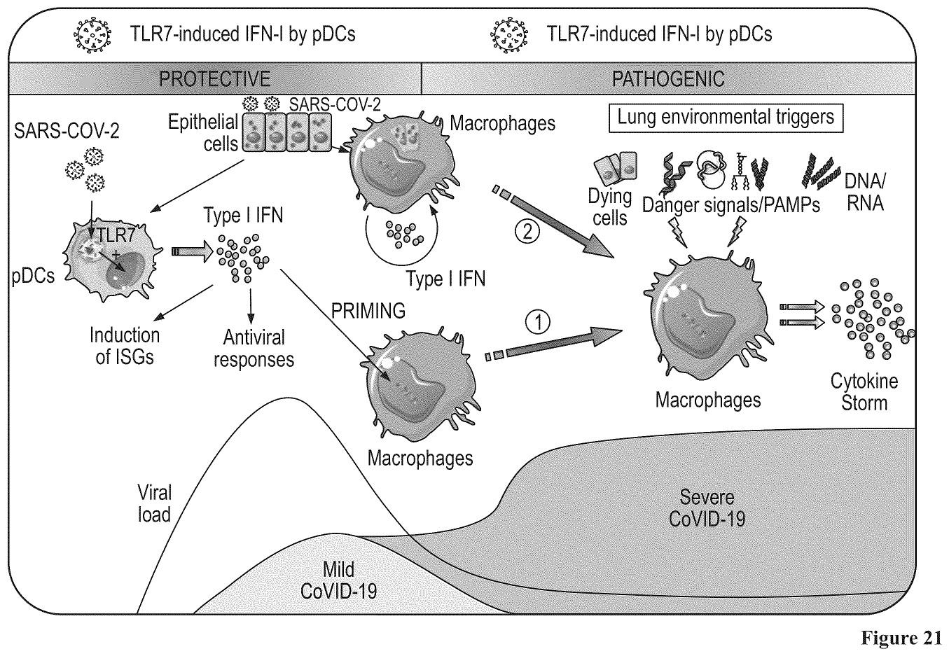

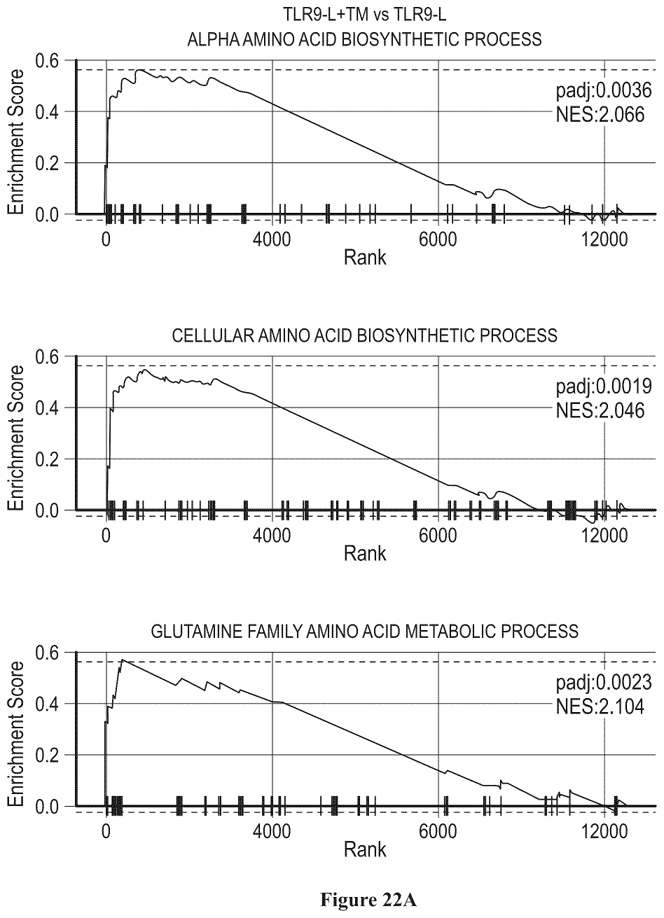

Combined with the in vitro data of live SARS-CoV-2 inoculated pDCs, these data support a scenario where the IFN-I is coming from at least 2 different sources—pDCs by directly sensing of the live virus and macrophages by interacting with epithelial cells infected by the virus (see model FIG. 21).

### Example 3. The Production of IFN-I by pDCs in Response to SARS-CoV-2 Superinduces Macrophage Responses to Environmental Stimuli

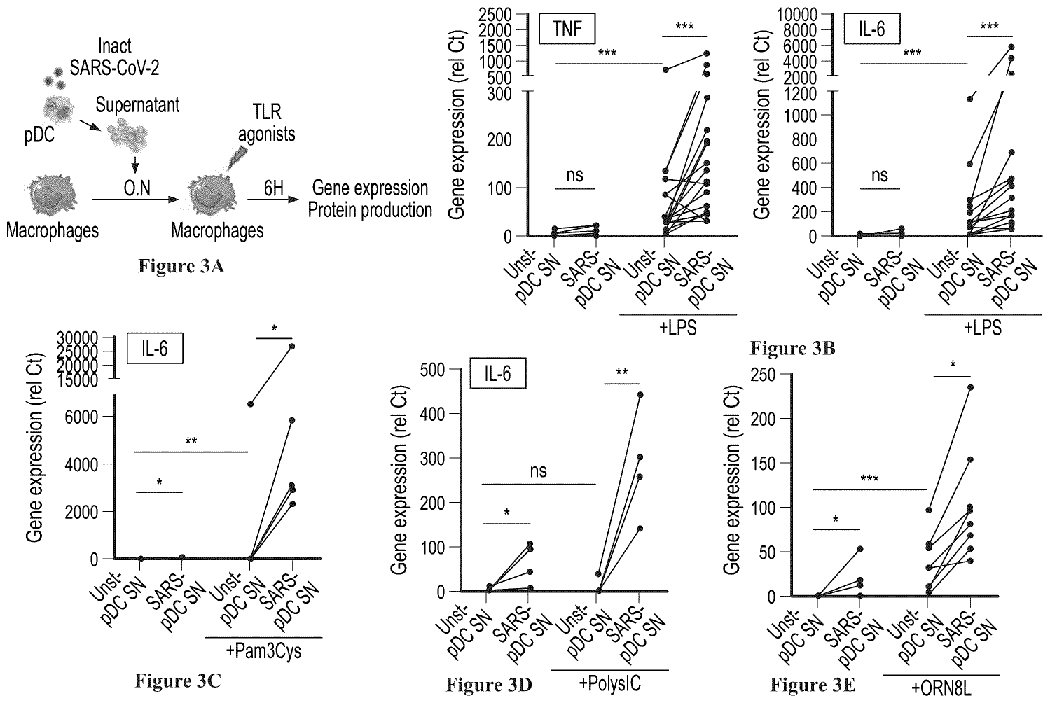

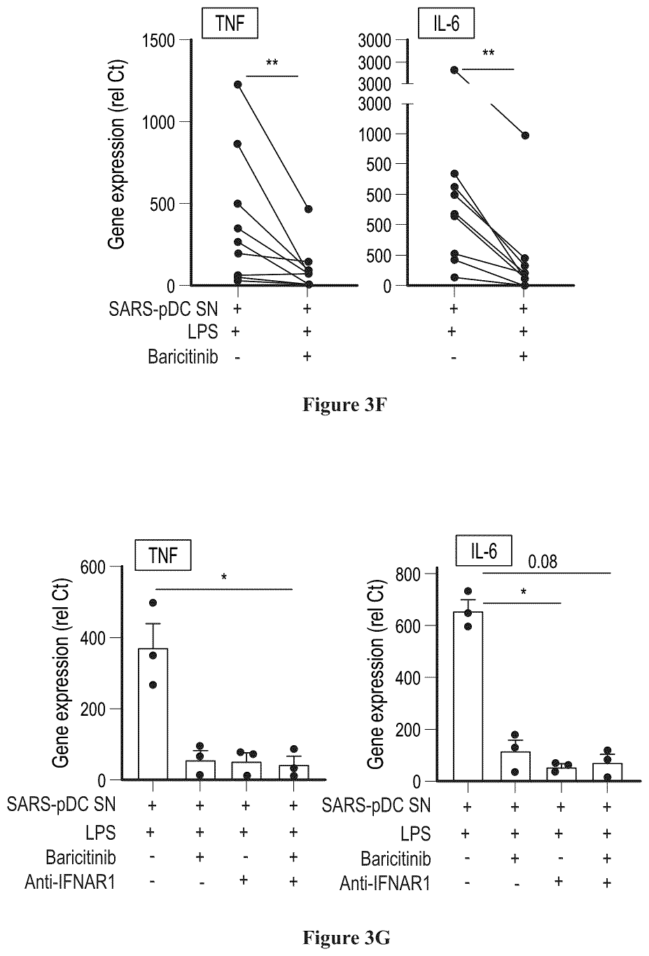

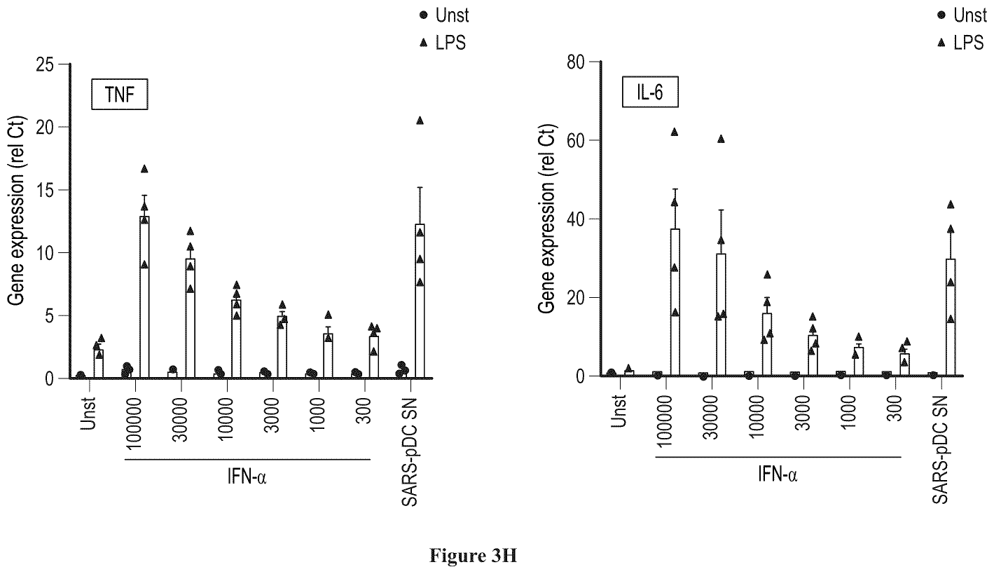

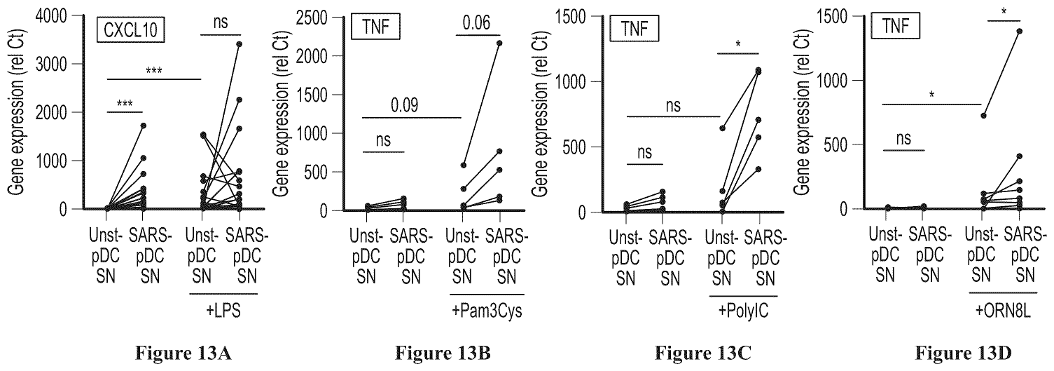

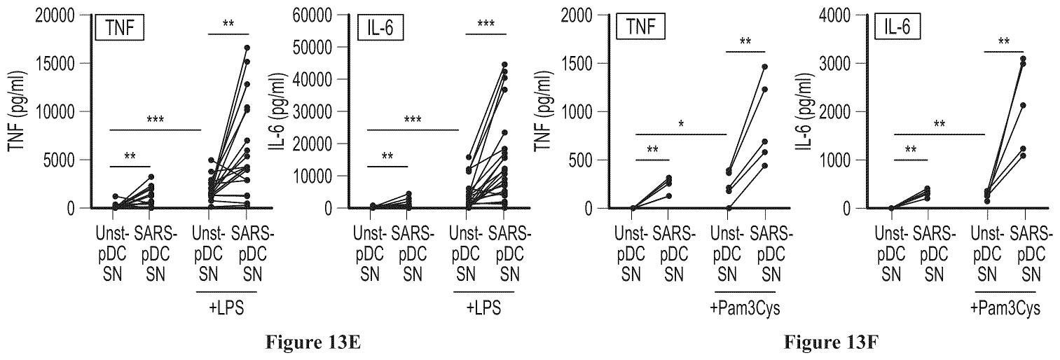

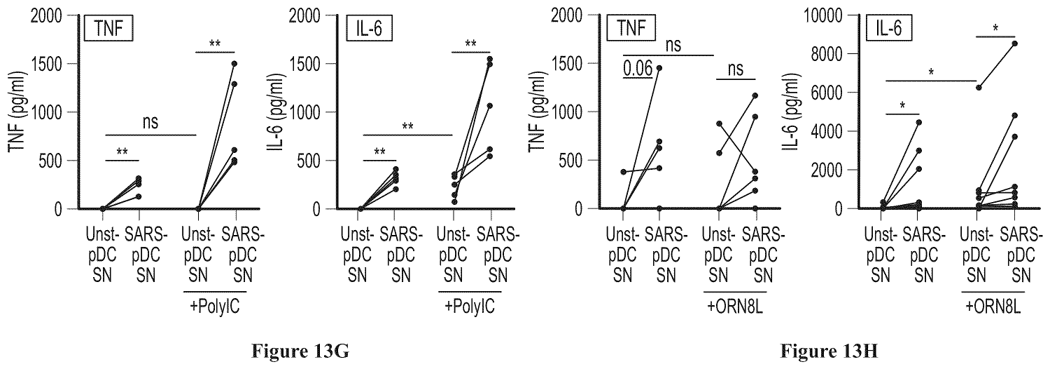

An increase in bacterial infections in patients with COVID-19, resulting in higher levels of bacterial products (bacterial DNA/RNA, lipoproteins and also of LPS) in ICU patients has been reported (Arunachalam, P. S. et al. Science 369, 1210-1220, (2020)). Thus, it was determined whether SARS-CoV-2 activated pDCs, especially IFN-I induced in these cells, could impact macrophage response into the secretion of the so-called cytokine storm (Rendeiro, A. F. et al. Nature 593, 564-569, (2021)). Supernatants of SARS-pDCs had little impact when used alone but drastically amplified the production and expression of proinflammatory cytokines such as TNF and TL-6 by macrophages in response to LPS but also to Pam3Cys (agonist of another transmembrane TLR), Poly I.C, and the TLR8 agonist ORN8L (both being RNA-sensing TLRs which are endosomal) (FIGS. 3A-3E and FIGS. 13B-13H). CXCL10 was induced in macrophages by SARS-pDCs, likely due the presence of IFN-I in the pDC supernatants and the addition of LPS had little effect (FIG. 13A). It was then tested whether supernatants from SARS-pDCs would also increase macrophage responses to SARS-CoV-2 itself. As shown in (FIGS. 12E-12F), there was little to no production or expression of IL-6 or TNF in these treated macrophages by the virus. However, live virus in the context of the inflammatory milieu of the lung may enhance the sensing and inflammatory response by macrophages.

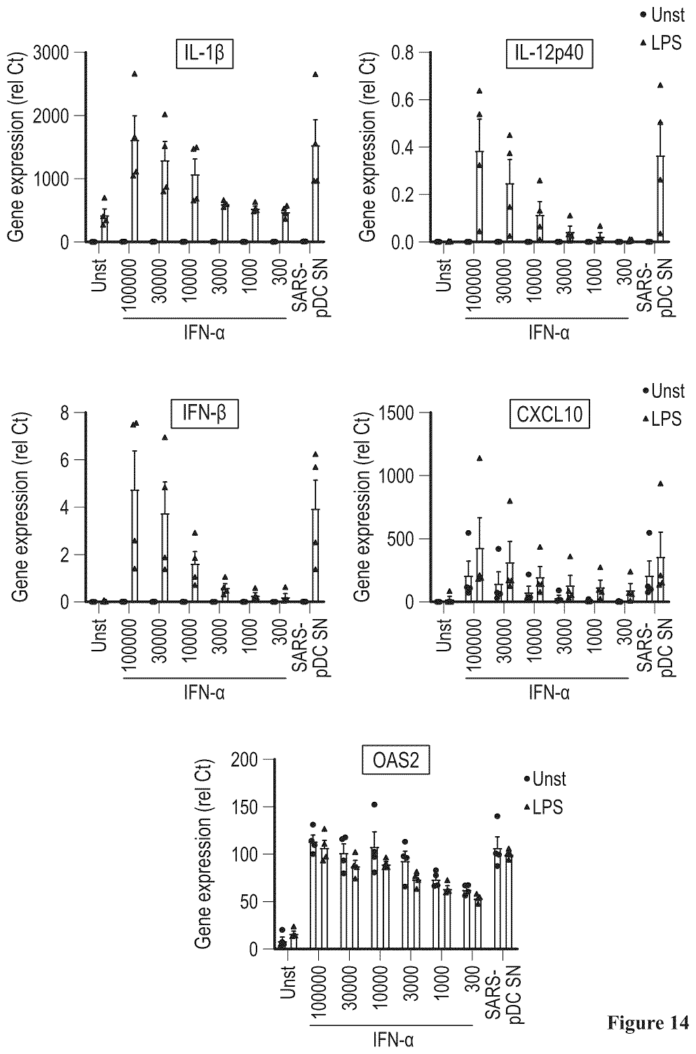

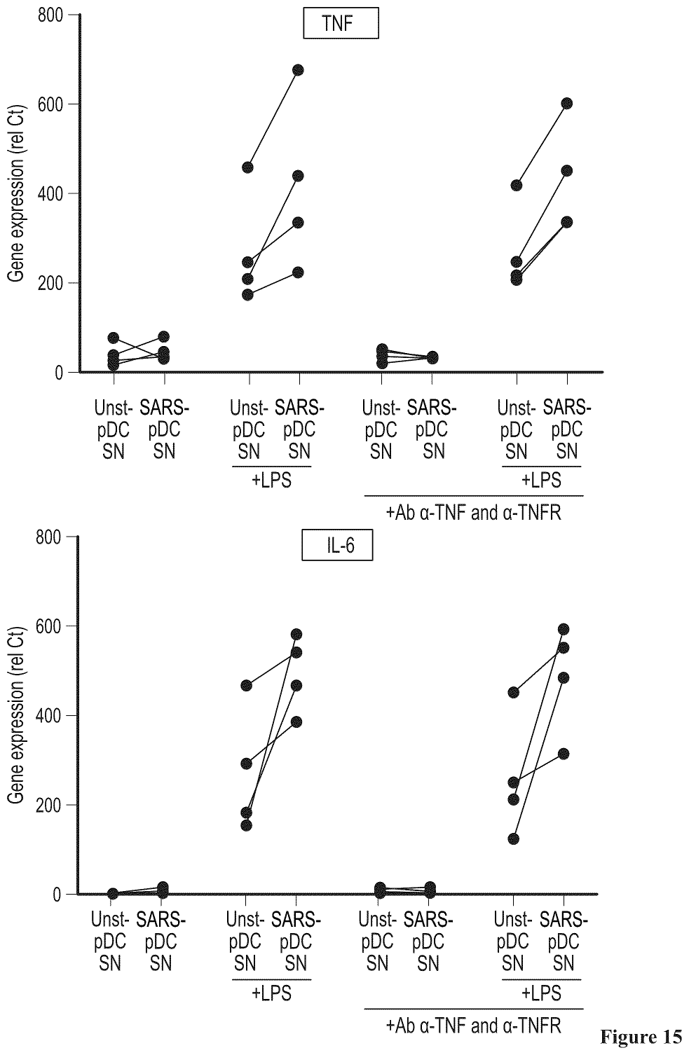

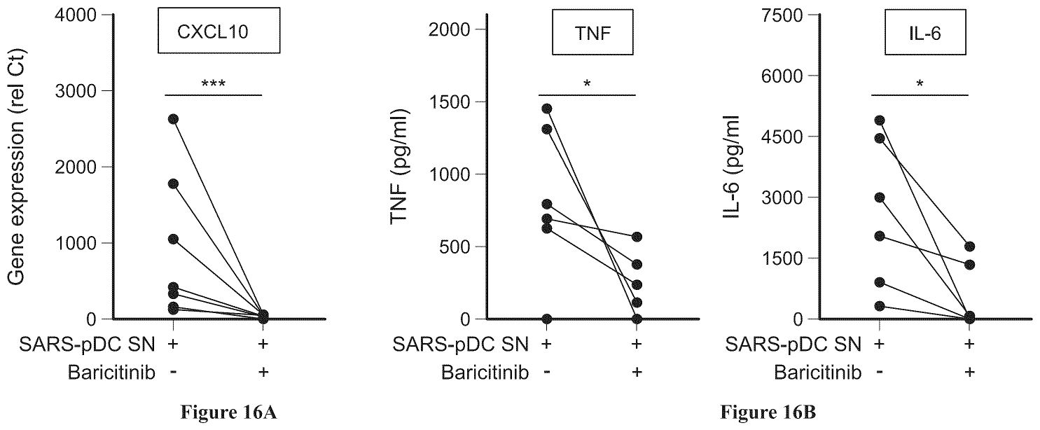

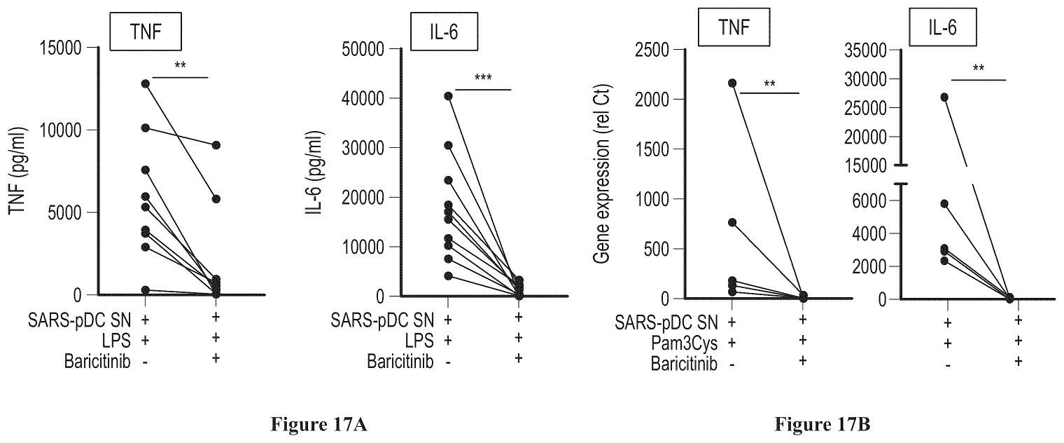

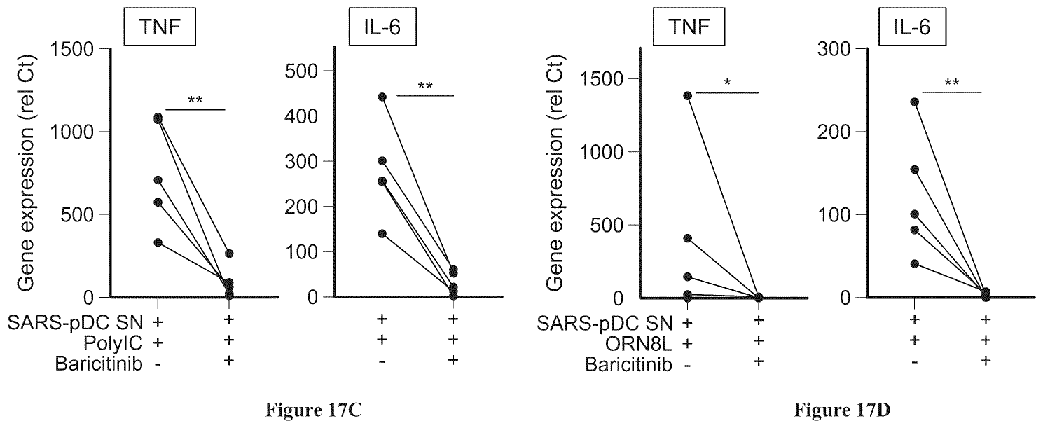

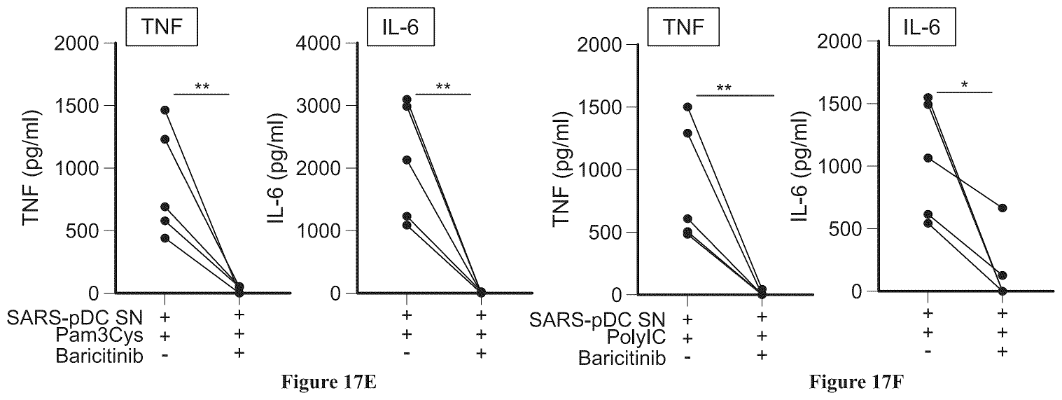

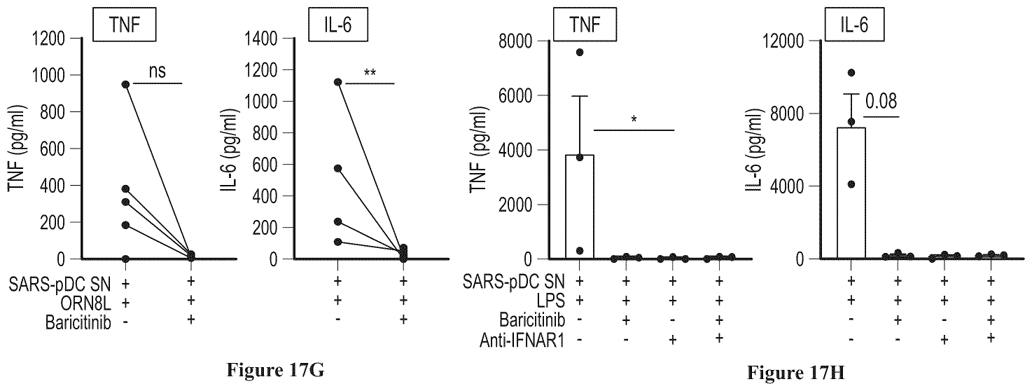

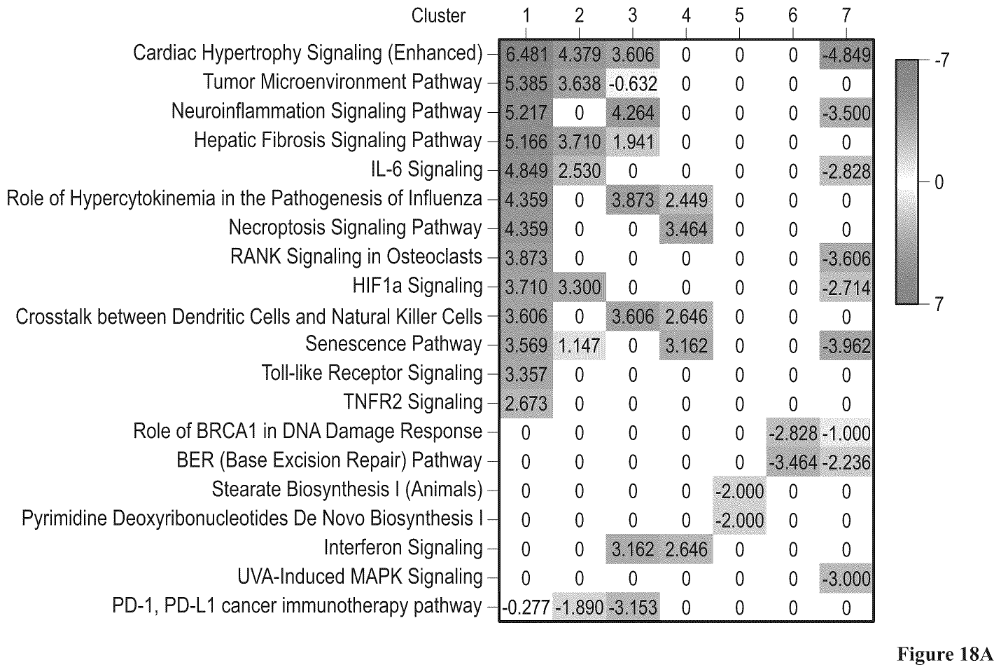

As pDCs produce large amounts of IFN-I in response to SARS-CoV-2 (FIG. 2A), macrophages were incubated with titrated amounts of IFN-α followed by LPS. A dose-dependent effect of IFN-α on the LPS-induced response was observed in macrophages with superinduction of TNF, IL6, IL1B, IL12B and IFNB after treatment with high concentrations of IFN-α, comparable to that observed with supernatants from SARS pDCs (FIG. 3H and FIG. 14). The impact of TNF which could have a synergistic or antagonistic effect to IFNα was also explored. However, blocking TNF or the TNFR was found to have no significant influence on macrophages primed with supernatants from SARS-pDCs in response to LPS (FIG. 15), although it is possible that TNF may play a role in the context of inflamed lung where TNF is abundantly secreted by the macrophages. Baricitinib inhibits JAK1/2 which is essential, although not restricted to IFNAR signaling (Ivashkiv, L. B. & Donlin, L. T. Nat Rev Immunol 14, 36-49, (2014) and early evaluation in combination with remdesivir in hospitalized patients with COVID-19 yielded promising results with significantly reduced mortality (Kalil, A. C. et al. N Engl J Med 384, 795-807, (2021). Baricitinib prevented the induction of CXCL10 but also of TNF and IL-6 secretion in macrophages cultured with the supernatant of SARS-pDCs (FIGS. 16A-16B). Strikingly, inhibiting JAK1/2 prevented the superinduction of TNF and IL-6 by all the stimuli used, LPS, Pam3Cys, Poly I:C or ORN8L in macrophages exposed to SARS-pDC supernatants (FIG. 3F and FIGS. 17A-17G). Blocking the IFNAR had a similar effect (FIG. 3G and FIG. 17H) suggesting that the effect is due to IFN-I. In conclusion, macrophages produce exacerbated amount of proinflammatory cytokines in response to environmental stimulus when exposed to IFN-I from SARS-pDCs.

### Example 4. The Production of IFN-I by pDCs in Response to SARS-CoV-2 Mediates Epigenetic and Transcriptional Changes in Macrophages

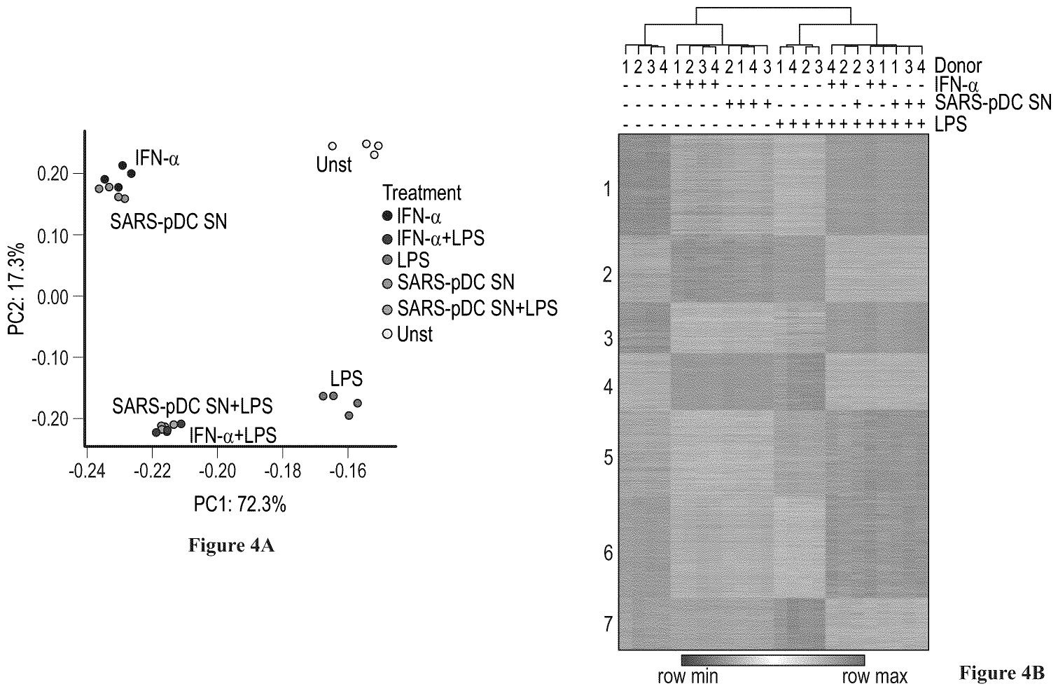

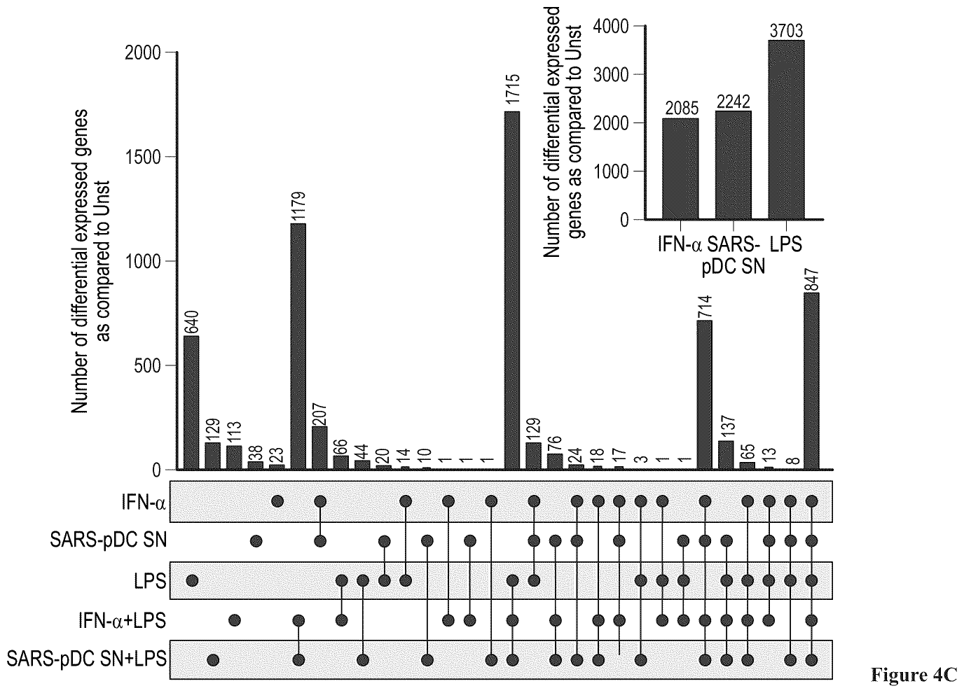

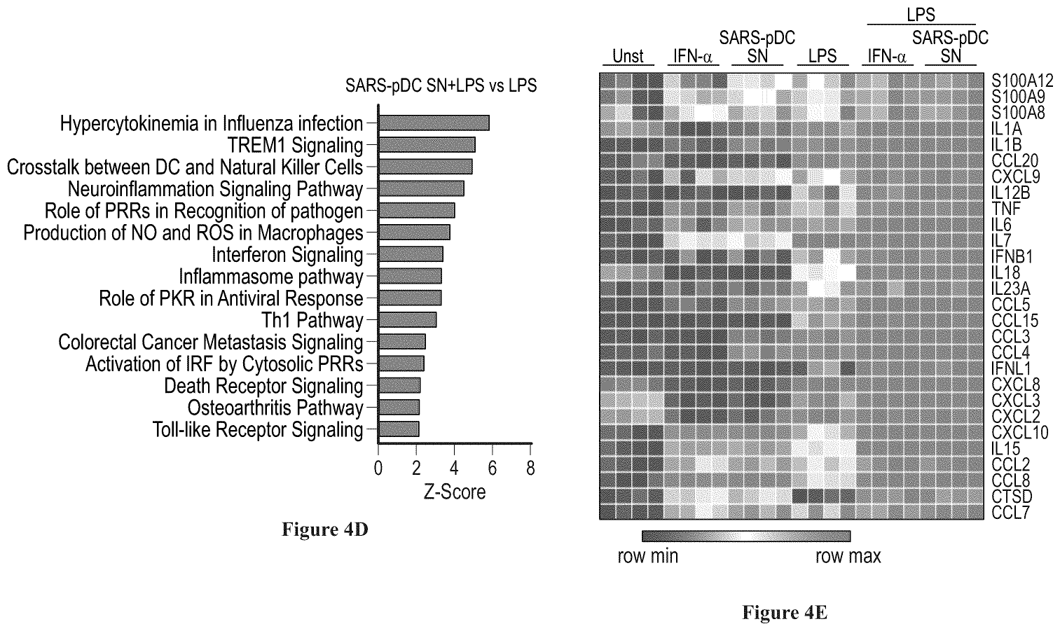

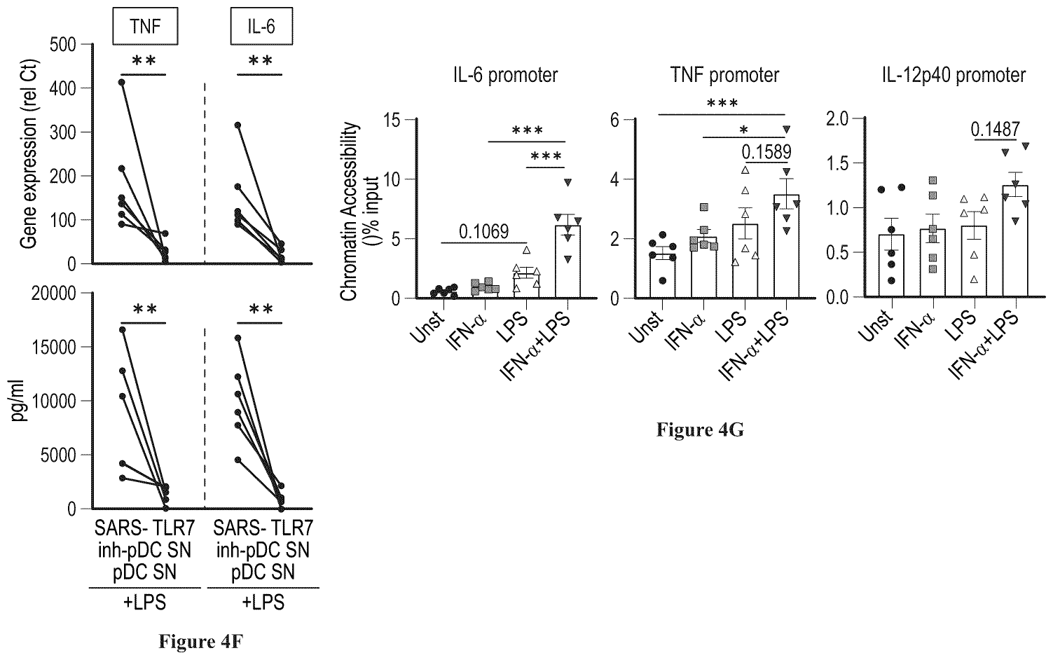

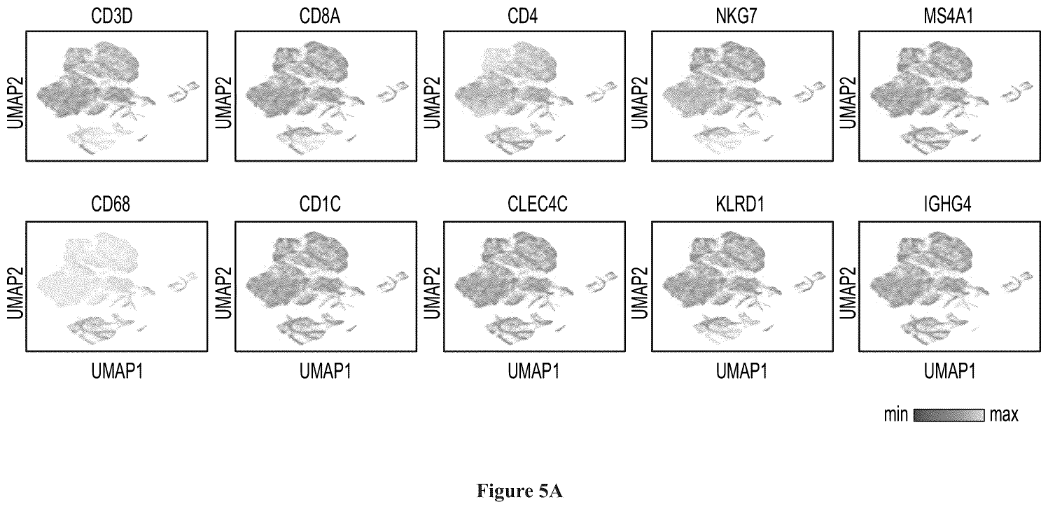

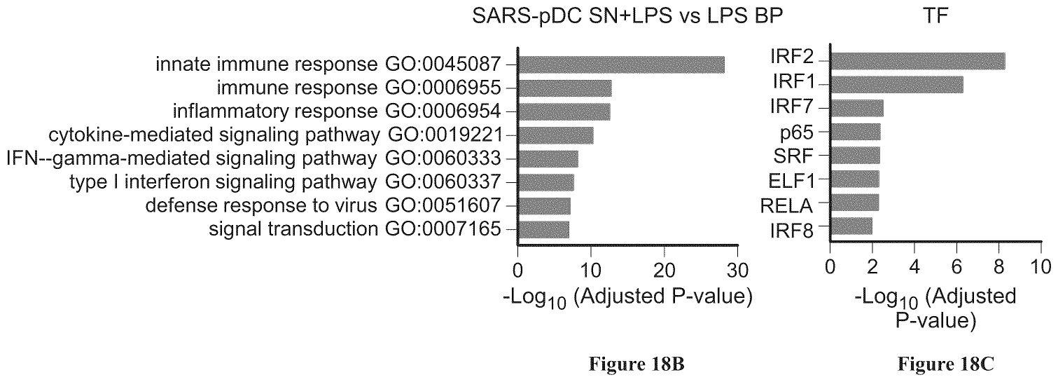

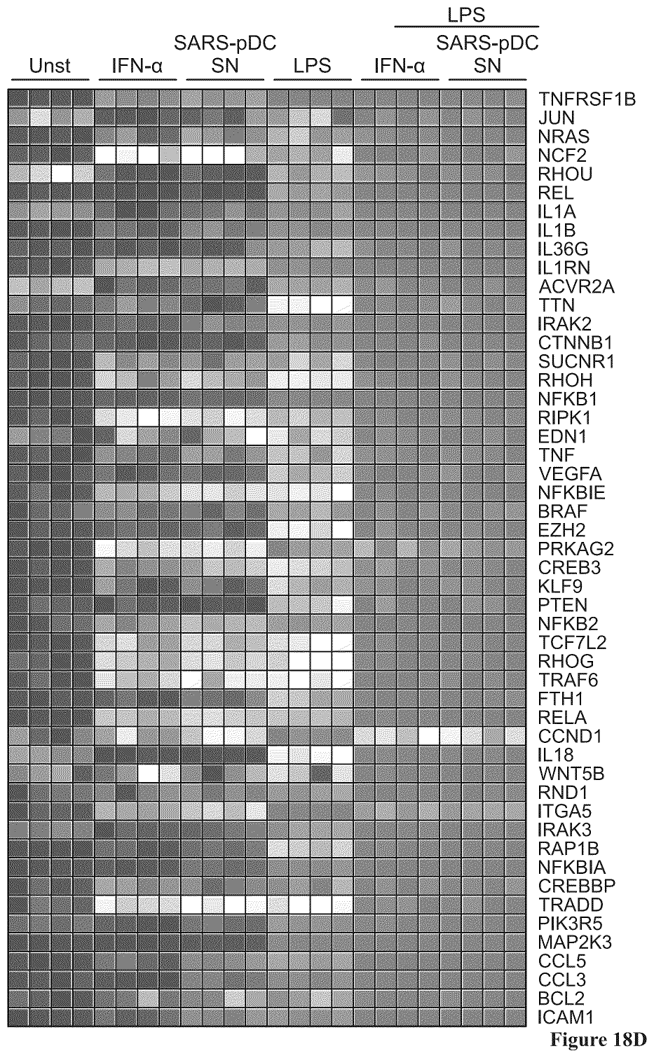

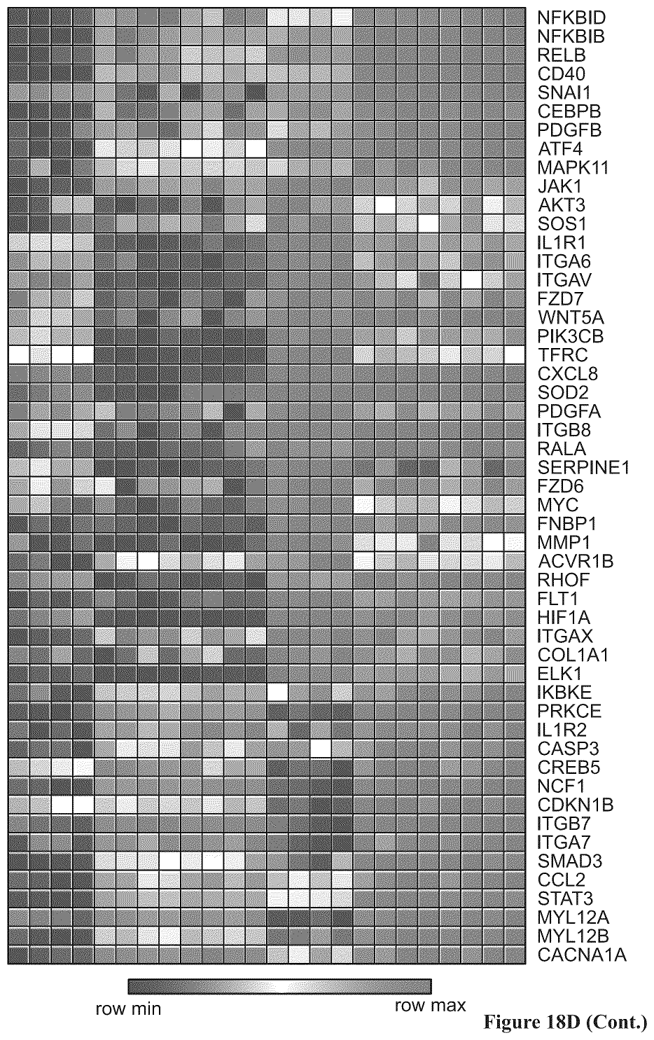

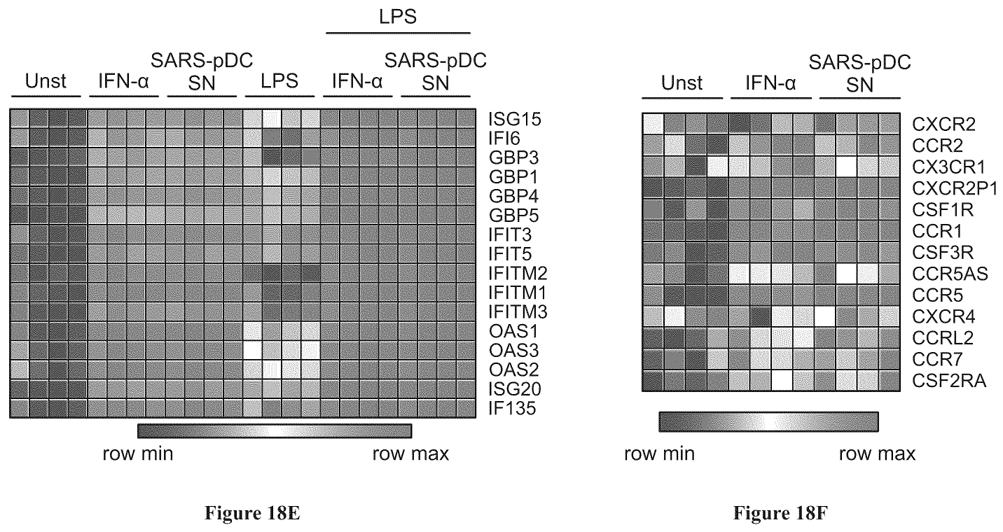

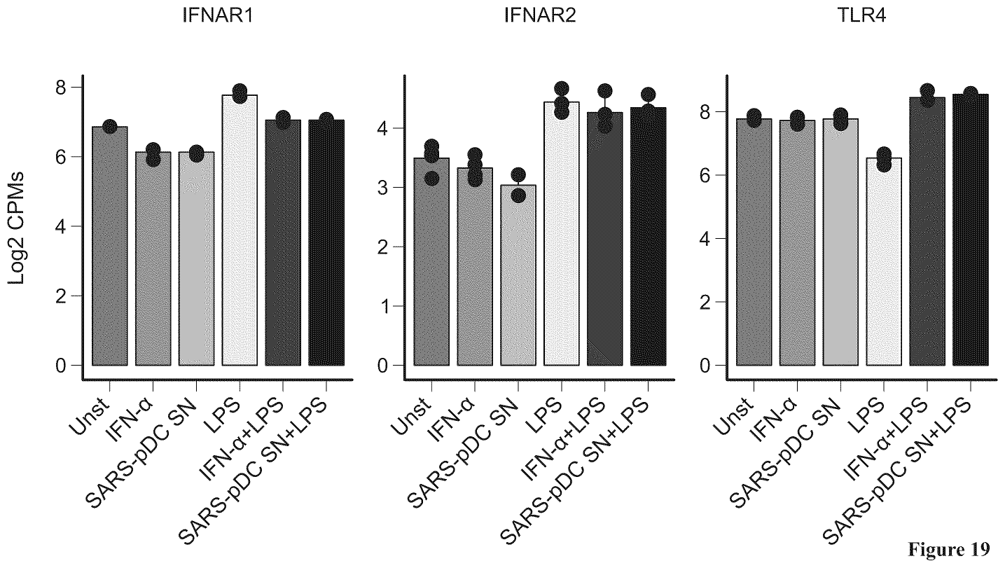

To obtain a comprehensive understanding of how type I IFN and SARS-pDC supernatants influence macrophage activation, transcriptomic analysis was performed using RNA-seq to evaluate TLR4 responses in macrophages exposed with IFN-α or SARS-pDC supernatants. Principal component analysis (PCA) showed that pDC supernatants and IFN-α conditions closely clustered together (FIG. 4A); IFN-α- and SARS-pDC supernatant-induced genes (differentially expressed genes or DEGs, FDR <0.05 and fold-induction >2) highly overlapped with commonly induced genes (FIGS. 4B-4C). Similarly, macrophages pre-incubated with either IFN-α or SARS-pDC supernatants and stimulated with LPS tightly clustered together, clearly separated from the LPS alone condition (FIG. 4a); 92% of DEGs induced by LPS in IFN-α- or SARS pDC supernatant-treated macrophages were common (FIG. 4C). The data show that both LPS and IFN-α/SARS-pDC supernatants contributed to the changes in gene expression (FIG. 4C). Of note, IFN-α and SARS-pDCs supernatants had a significant impact on chemokine receptors expression on macrophages (FIG. 18F), suggesting a role of IFN-α in promoting the infiltration of macrophages to the lungs of patients. In contrast, there was little change on TLR4 expression or of the 2 subunits of the IFNAR in the macrophages (FIG. 19). K-means clustering segregated DEGs into 7 groups based on patterns of expression (FIG. 4B). Notably, cluster 1 was comprised of LPS-inducible genes that were superinduced by IFN-α or SARS-pDC supernatants (similar to TNF and IL6), whereas cluster 3 was comprised of ISGs that were superinduced by LPS. Cluster 1 included many genes encoding pro-inflammatory cytokines and chemokines (FIG. 4E and FIG. 18A), and fibrosis relate genes (FIG. 18D), which are implicated in COVID-19 pathogenesis (Melms, J. C. et al. Nature 595, 114-119, (2021); Zhu, L. et al. Immunity 53, 685-696 e683, (2020); Lucas, C. et al. Nature 584, 463-469, (2020). Additional bioinformatic analysis comparing macrophages stimulated with SARS-pDC supernatants versus LPS alone revealed enrichment for inflammatory and immune pathways (FIG. 4D and FIG. 18B). Ingenuity Pathway Analysis suggested a role for IRF and NF-κB family transcription factors in the superactivation of inflammatory genes by IFN-α or SARS-pDC supernatants with LPS (FIG. 18c), which is in line with a previous report (Park, S. H. et al. Nat Immunol 18, 1104-1116, (2017)). Unsurprisingly, cluster 3 showed enrichment of IFN signaling (FIG. 18A) and was comprised of canonical ISGs (FIG. 18E). Overall, these results support a role for pDC-derived IFN-I in superinduction of a TLR4-mediated inflammatory response.

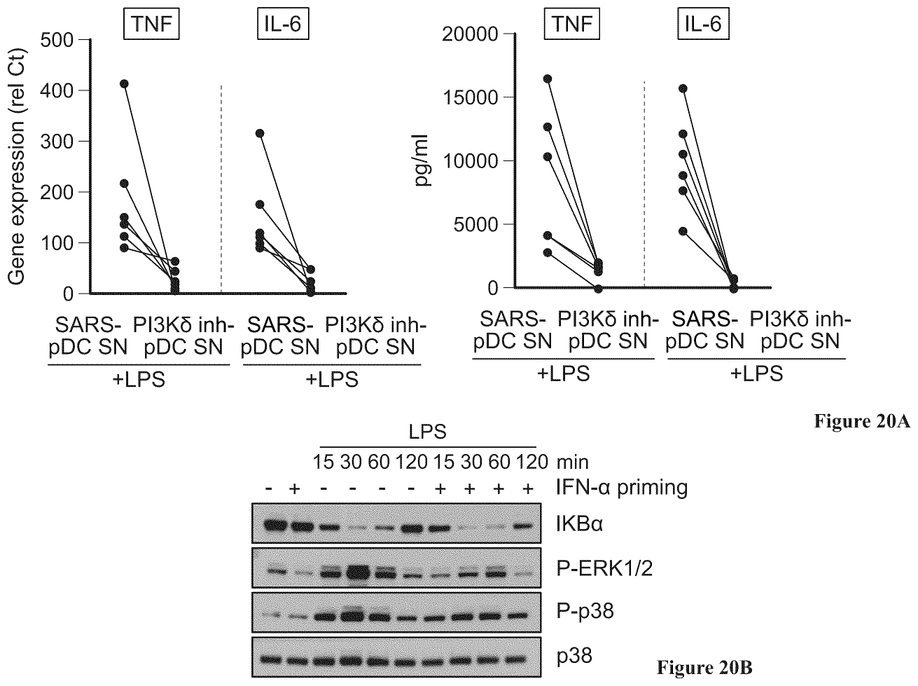

Accordingly, inhibition of IFN-I production using either the TLR7 inhibitor IRS661 or the PI3Kδ inhibitor CAL-101 reduced inflammatory gene induction upon challenge with LPS (FIG. 4F and FIG. 20A). IFN-α had minimal effects on TLR4-induced IKBα degradation or activation of MAPKs ERK and p38 (FIG. 20B), which is in accordance with previous work (Park, S. H. et al. Nat Immunol 18, 1104-1116, (2017)). However, cells primed with IFN-α and then followed by LPS treatment significantly increased chromatin accessibility of IL6 and TNF promoters (FIG. 4G). In a previous report (Qiao, Y. et al. Immunity 39, 454-469, (2013), it was shown that the concentrations of IFN-I (200 antiviral units/ml) that are capable of fully inducing antiviral ISG expression (‘IFN signature’ and by inference an antiviral response) did not augment subsequent TLR responses while further observation demonstrated that higher concentrations of IFN-α prevented TNF-induced tolerance of a subset of TLR4-inducible genes (Park, S. H. et al. Nat Immunol 18, 1104-1116, (2017)). The data here showed that the extraordinarily high concentrations of IFN-I produced by SARS-pDCs (FIG. 2A) reprogram macrophages for an augmented hyperinflammatory TLR response. Thus, low concentrations of IFN-I induce antiviral responses without inflammatory toxicity, but high pDC-derived IFN-Is additionally promote the cytokine storm. Hence, these data demonstrate that IFN-I produced by pDCs in response to SARS-CoV-2 mediates transcriptional and epigenetic changes in macrophages which exacerbate their production of inflammatory mediators in response to environmental triggers.

### Example 5. Fueling the TCA Cycle and ATP Generation are Required for TLR9-Induced IFN-I Response by pDCs

The spliced XBP1 isoform generated by IRE1a encodes the functional transcription factor XBP1, which induces factors implicated in restoring ER proteostasis while controlling diverse metabolic programs (25, 26).

For the following experiments, enriched leukocytes were obtained from New York blood center (Long Island City, NY) under internal Institutional Review Board-approved protocols. PBMCs were prepared using Ficoll-Paque density gradient and pDCs were isolated using BDCA4+ positive selection (Miltenyi Biotech: 130-097-415) as previously described (42). pDCs were cultured at 40,000 cells (for healthy donors) per well in a 96-round bottom plate and incubated at 37° C., 5% CO2 and 95% humidity. For TLR7 and TLR9 activation assay, pDCs were stimulated with heat-inactivated 2 μMOI of H1N1 VR-95 influenza A virus (ATCC) and 0.075 μM of C274 (42) respectively.

In some culture conditions, cells were cultured with the tunicamycin (thermofisher: 654380), thapsigargin (Sigma: T9033), 4 μ8c (EMD Millipore: 412512), MKC8866 (Medchem Express: HY-104040), IXA4 (Chembridge: 131171.1), AMG PERK44 (R&D: 5517), Ceapin-A7 (Sigma: SML2330), NCT-503(Axon Medchem: 2623), L-serine (EMD Millipore: S4500), sodium pyruvate (Sigma: 8636), α-ketoglutaric acid disodium salt hydrate (Sigma: K3752), CPI-613 (Selleckchem: S2776), Anti-PF4 antibody (Abcam: ab9561), CXCL4 (Sigma: SRP3142).

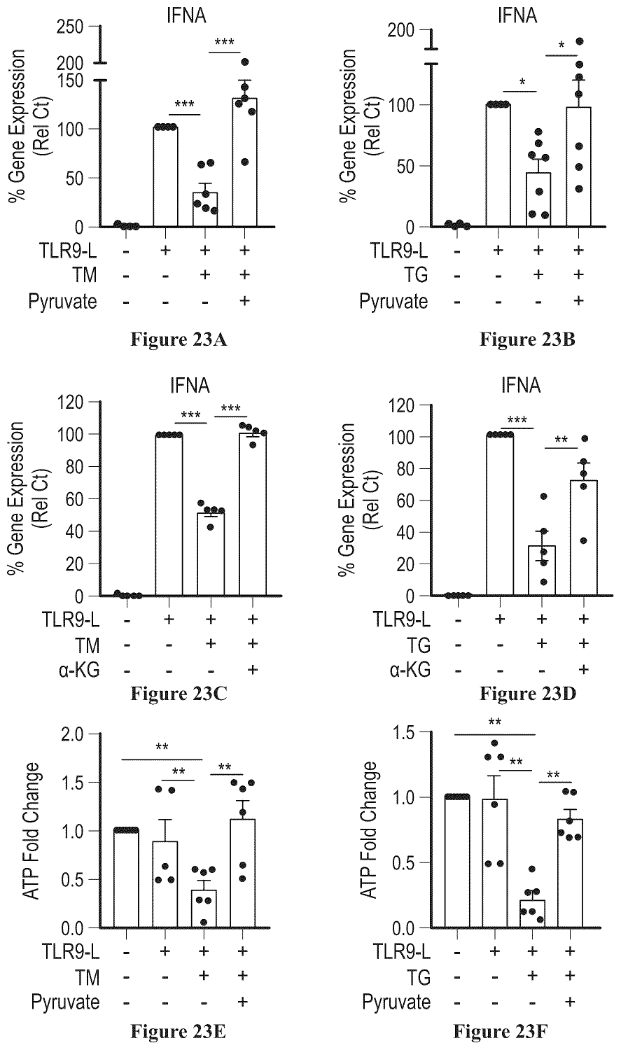

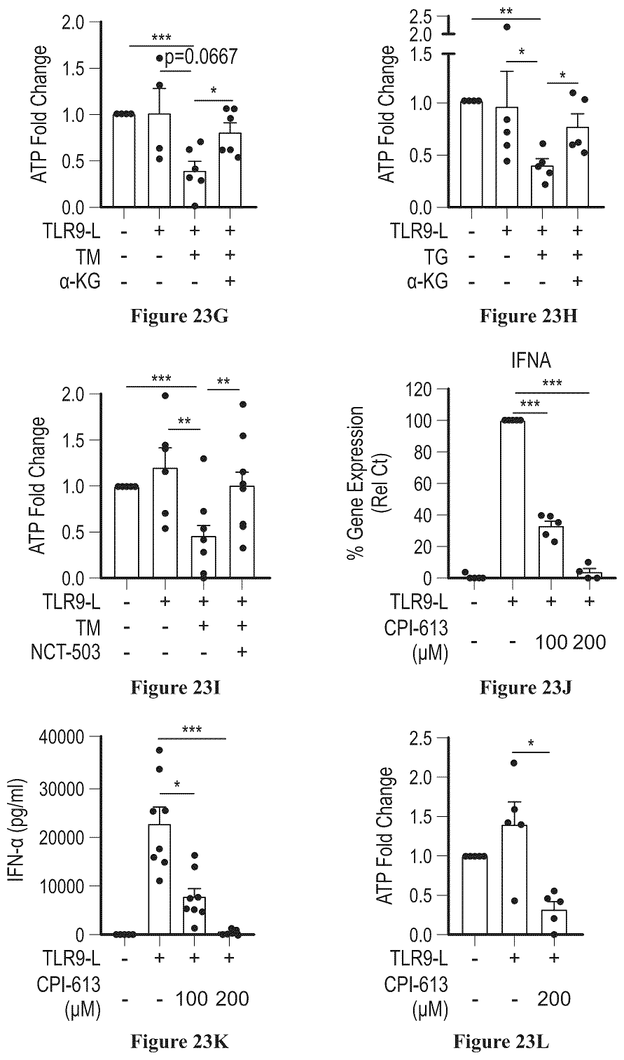

Using gene set enrichment analysis (GSEA), it was observed that transcriptional networks implicated in amino acid biosynthesis were markedly activated in pDCs experiencing ER stress, with or without TLR9 agonist treatment (FIGS. 22A and 22B). Further analysis showed that gene programs related to serine amino acid biosynthesis are highly enriched among all amino acid pathways (FIG. 22C). The induction of some of these genes by both tunicamycin or thapsigargin was confirmed, irrespective of TLR9 signaling (FIG. 22D). Of particular interest, tunicamycin or thapsigargin treatment markedly induced the gene encoding phosphoglycerate dehydrogenase (PHGDH) in pDCs (FIG. 22D). This enzyme transforms 3-phosphoglycerate into phosphohydroxypyruvate, which subsequently converts into serine (FIG. 22G) via transamination and phosphate ester hydrolysis reactions driven by PSAT1 and PSPH, respectively (27-29). Thus, a direct link was established between IRE1α-XBP1 and te induction of these metabolic regulated genes, as ER stress-driven induction of PHGDH (FIG. 22E) as well as PSAT1, and PSPH (data not shown) was markedly inhibited upon abrogation of IRE1α-XBP1 signaling and was conversely induced in pDCs treated with IXA4 (FIG. 22F). The impact of the IRE1α-XBP1-induced expression of PHGDH on pDCs activation was evaluated by inhibiting PHGDH enzymatic activity. Abrogating PHGDH activity using NCT-503 (30) restored IFNA expression by TLR9-activated pDCs facing ER stress (FIGS. 22H and 22I). Since increased expression of PHGDH can boost serine biosynthesis, it may also cause a deficiency in pyruvate levels by shunting glycolysis (FIG. 22G) and the impact of both pathways on pDCs activity was evaluated. First, it was observed that IFN-α secretion by TLR9-activated pDCs was unaltered upon exogenous supplementation with L-serine (FIG. 22J), suggesting that elevated L-serine is not involved in ER stress-mediated IFN-α inhibition. In contrast, the intracellular pyruvate levels were significantly reduced in ER stressed-pDCs, and these levels could be restored by blocking PHGDH activity (FIGS. 22K and 22L). Further confirming these observations, exogenous pyruvate supplementation was sufficient to restore the IFNA expression by pDCs under ER stress condition (FIGS. 23A-23B). When produced by the cells, pyruvate enters the mitochondrion to fuel the tricarboxylic acid cycle (TCA) where it is converted to α-ketoglutarate (α-KG) and other TCA cycle substrates, to ultimately produce ATP by the electron transport chain (31-33) and this process has been shown to contribute to immune cell activation (34). It was observed that treatment with a cell-permeable analog of α-KG (35) also rescued the IFNA expression by TLR9-activated pDCs undergoing ER stress (FIGS. 23C-23D). Consistent with these findings, it was observed that ER stress reduced intracellular ATP levels (FIGS. 23E-23H) and that supplementing either pyruvate (FIGS. 23E-23F) or α-KG (FIGS. 23G-23H) reversed that inhibition and restored the ATP levels. Of note, this was directly linked to increased expression of PHGDH during ER stress, as blocking its activity with NCT-503 similarly restored the intracellular ATP levels (FIG. 23I).

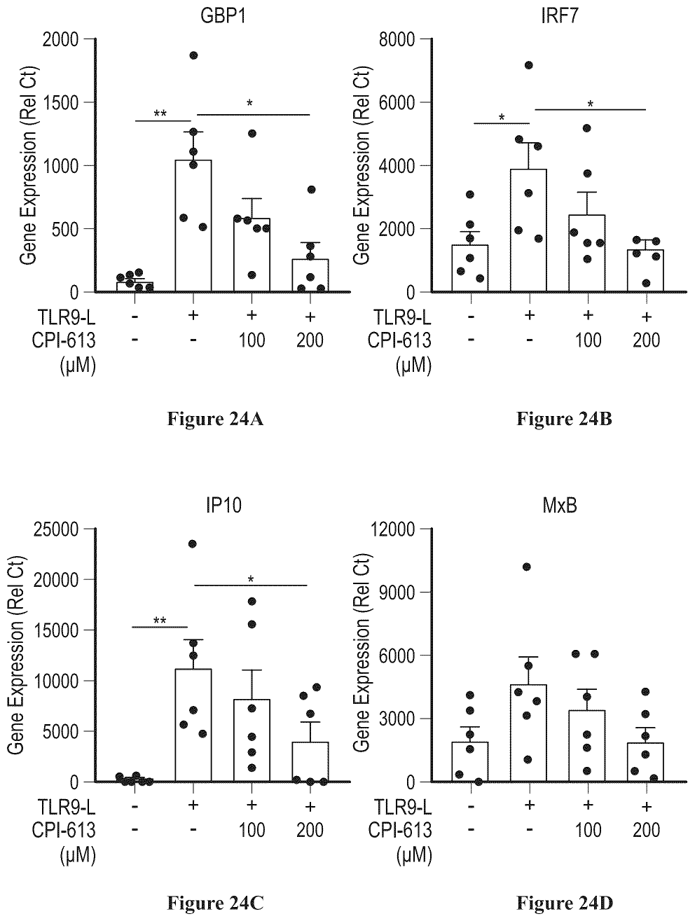

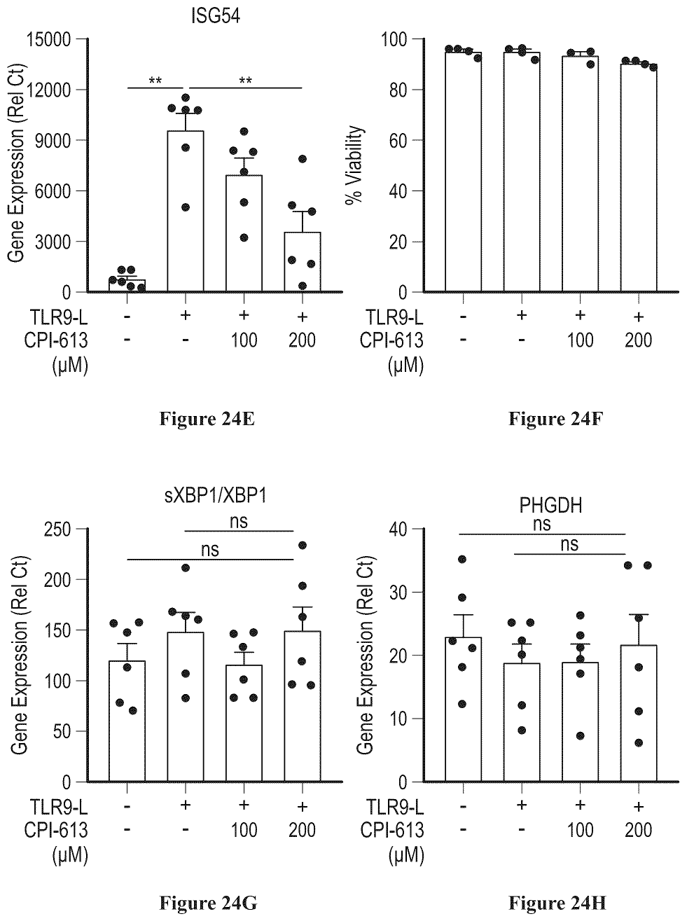

Using an inhibitor of both α-ketoglutarate dehydrogenase (KGDH) and pyruvate dehydrogenase (PDH), called CPI-613 (6,8-bis-benzylthio-octanoic acid), it was tested whether disrupting the TCA cycle could impact the IFN-α response by pDCs. CPI-613 has been well characterized and is in clinical trials for pancreatic cancer (36, 37). As shown in FIGS. 23J-23K, CPI-613 inhibited the expression and secretion of IFN-α. As shown in FIGS. 24A-24E, CPI-613 also inhibited the expression of ISGs such as GBP1, IRF7, ISG54, MxB, CXCL10, while it had no effect on cell viability (FIG. 24F), or on the expression of PHGDH or on XBP1 splicing (FIGS. 24G-24H). Consistent with this data, CPI-613 also reduced intracellular ATP levels in TLR9-activated pDCs (FIG. 24L).

Collectively, these data indicate that pyruvate and α-KG are key intermediate metabolites in the TCA cycle that are required for optimal IFN-α responses in TLR9-activated pDCs, and that this process is markedly blunted upon ER stress-driven activation of RE1-XBP1 signaling due to the increased activity of PHGDH.

### Example 6. TCA Inhibitors UK5099 and CB839 (Telagenastat) Inhibit Production of IFNA

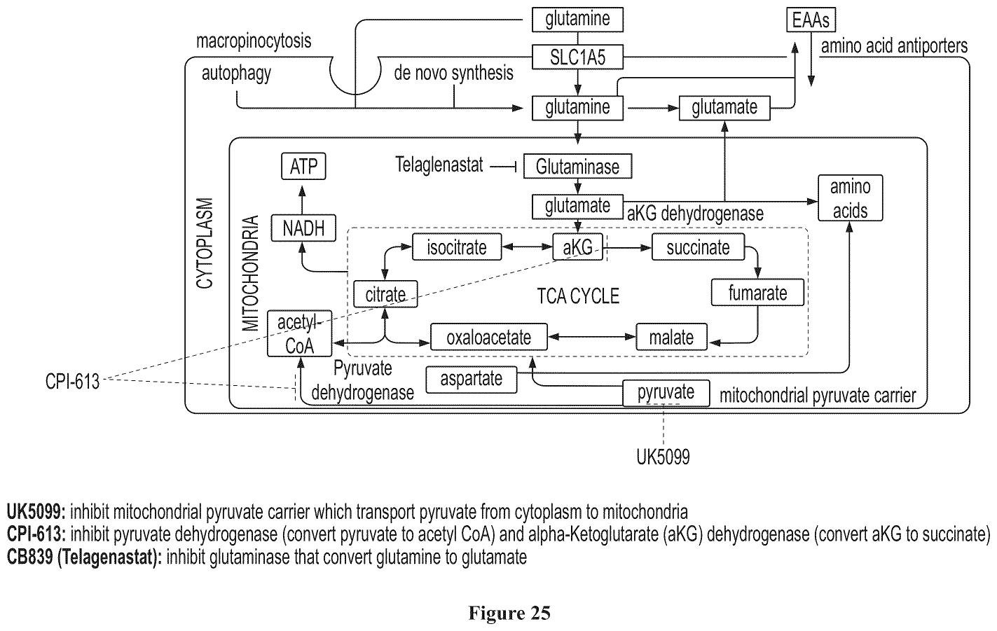

It is expected that a TCA inhibitor will reduce IFNA expression in the lungs of COVID-19 subjects and in pDCs isolated from the lungs of COVID-19 subjects. Inhibitors of the TCA cycle are shown in FIG. 25. Purified pDCs from Healthy Donors (HDs) and pDCs from COVID-19 subjects will be cultured with medium only or with various TCA inhibitors. pDCs cultured from COVID-19 subjects are expected to produce greater quantities of IFNA. TCA inhibitors (e.g., an inhibitor for pyruvate transporter (UK5099 at 10, 20, and 40 μM) or an inhibitor of glutaminase (CB839 at 0.5 μM) or CPI-613 are expected to reduce IFNA expression in cultured pDCs.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a condition selected from the group consisting of a viral disease or hypercytokinemia in a human subject in need thereof, the method comprising administering to the subject a therapeutically effective amount of a compound that disrupts the tri-carboxylic acid (TCA) cycle in immune cells or a compound that activates the Unfolded Protein response (UPR) in immune cells in the subject.

2. The method according to claim 1, wherein the hypercytokinemia comprises an overproduction of immune cells and pro-inflammatory cytokines into the lungs of the subject.

3. The method of claim 1, wherein the compound that activates the UPR activates the IRE1α-XBP1 signaling branch of the UPR in immune cells.

4. The method of claim 1, wherein the compound that activates the UPR is tunicamycin, or thapsigargin.

5. The method of claim 1, wherein the compound that activates the UPR is IXA4.

6. The method of any one of claims 1-5, wherein the immune cells are dendritic cells, macrophages, T cells, B cells, natural killer cells, and/or neutrophils.

7. The method of claim 1, wherein the compound that disrupts the tri-carboxylic acid (TCA) cycle is
(a) a compound of Formula I 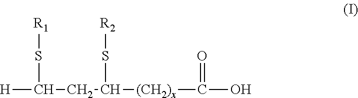
or a pharmaceutically acceptable salt thereof,
wherein R₁ and R₂ are independently selected from the group consisting of acyl defined as R₃C(O)—, alkyl defined as CₙH₂ₙ₊₁, alkenyl defined as CₘH₂ₘ₋₁, alkynyl defined as CₘH₂ₘ₋₃, aryl, heteroaryl, alkyl sulfide defined as CH₃(CH₂)ₙ—S—, imidoyl defined as R₃C(═NH)—, hemiacetal defined as R₄CH(OH)—S—, and hydrogen provided that at least one of R₁ and R₂ is not hydrogen; wherein R₁ and R₂ as defined above can be unsubstituted or substituted; wherein R₃ is hydrogen, alkyl, alkenyl, alkynyl, cycloalkyl, aryl, alkylaryl, heteroaryl, or heterocyclyl, any of which can be substituted or unsubstituted; wherein R₄ is CCl₃ or COOH; and wherein x is 0-16, n is 0-10 and m is 2-10,
(b) UK5099 (PF-1005023) 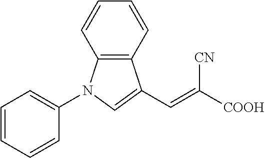 [SMILES: N#C/C(=C\c1cn(-c2ccccc2)c2ccccc12)C(=O)O]
or (c) CB839 (Telagenastat)

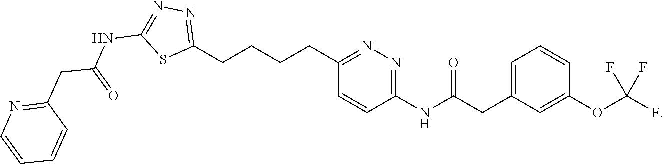 [SMILES: O=C(Cc1cccc(OC(F)(F)F)c1)Nc1ccc(CCCCc2nnc(NC(=O)Cc3ccccn3)s2)nn1]

8. The method of claim 7, wherein R₁ and R₂ are benzyl or benzoyl.

9. The method of claim 7, wherein the compound of Formula I is

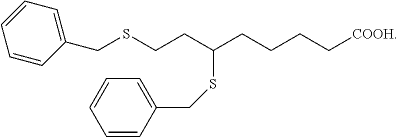 [SMILES: O=C(O)CCCCC(CCSCc1ccccc1)SCc1ccccc1]

10. The method of claim 7, wherein the compound of formula I is 6,8-bis-benzylthio-octanoic acid.

11. The method of claim 1, wherein the viral disease is coronavirus disease-19 (COVID-19), influenza, Severe Acute Respiratory Syndrome (SARS), or Hantavirus Pulmonary Syndrome (HPS).

12. The method of any one of the preceding claims, wherein the subject is concurrently treated with one or more agents selected from the group consisting of a corticosteroid, remdesivir, Nirmatrelvir, Bebtelovimab, Molnupiravir, an IL-6 inhibitor, an IL-1 inhibitor, a kinase inhibitor, a complement inhibitor, ivermectin, hydroxychloroquine, favipiravir, interferon-beta and a nonsteroidal anti-inflammatory drug (NSAID).

13. The method of claim 12, wherein the immunosuppressant is methotrexate, mycophenolate mofetil (MMF), cyclophosphamide, cyclosporin, or azathioprine.

14. The method of claim 13, wherein the corticosteroid is hydrocortisone, methylprednisolone, dexamethasone or prednisone.

15. The method of claim 1, wherein the treatment reduces production of inflammatory cytokines or chemokines by dendritic cells in the human subject.

16. The method of claim 15, wherein the inflammatory cytokines or chemokines are selected from the group consisting of: type I interferon (IFN-I), IL-6, or TNF-α, type III interferon, MIP-1a/CCL3, MIP-1/CCL4, CCL5/RANTES, and IP-10/CXCL10.

17. The method of claim 6, wherein the dendritic cells are plasmocytoid dendritic cells.

18. The method of claim 17, wherein the dendritic cells express one or more of CD123, CD303 (BDCA2), CD304 (BDCA4), and immunoglobulin-like transcript 7 (ILT7).

19. The method of claim 17, wherein the dendritic cells do not express the lineage-associated markers (Lin) CD3, CD19, CD14, CD16 and CD11c.

20. The method of claim 16, wherein the method inhibits and/or reduces IFN-I production in the human subject in need thereof by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, as compared to the corresponding reference levels in the human subject or in a control.

21. The method of claim 1, wherein the treatment reduces the expression of one or more of the interferon stimulated genes selected from the group consisting of Guanylate Binding Protein 1 (GBP1), Interferon Regulatory Factor 7 (IRF7), interferon stimulated gene 54 (ISG54), myxovirus resistance protein B (MxB), and 2′-5′-Oligoadenylate Synthetase 2 (OAS2).

22. The method of claim 1, wherein the treatment enhances expression of phosphoglycerate dehydrogenase (PHGDH), phosphoserine Phosphatase (PSPH), and phosphoserine Aminotransferase 1 (PSAT1).

23. Use of a therapeutically effective amount of a compound that disrupts the tri-carboxylic acid (TCA) cycle or a compound activates the Unfolded Protein response (UPR) in immune cells in the subject to treat a condition selected from the group consisting of a viral disease and hypercytokinemia in the subject.

24. Use according to claim 23, wherein the compound that disrupts the tri-carboxylic acid (TCA) cycle is
(a) a compound of Formula I 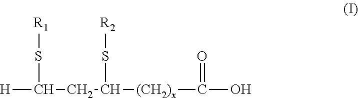
or a pharmaceutically acceptable salt thereof,
wherein R₁ and R₂ are independently selected from the group consisting of acyl defined as R₃C(O)—, alkyl defined as CₙH₂ₙ₊₁, alkenyl defined as CₘH₂ₘ₋₁, alkynyl defined as CₘH₂ₘ₋₃, aryl, heteroaryl, alkyl sulfide defined as CH₃(CH₂)ₙ—S—, imidoyl defined as R₃C(═NH)—, hemiacetal defined as R₄CH(OH)—S—, and hydrogen provided that at least one of R₁ and R₂ is not hydrogen; wherein R₁ and R₂ as defined above can be unsubstituted or substituted; wherein R₃ is hydrogen, alkyl, alkenyl, alkynyl, cycloalkyl, aryl, alkylaryl, heteroaryl, or heterocyclyl, any of which can be substituted or unsubstituted; wherein R₄ is CCl₃ or COOH; and wherein x is 0-16, n is 0-10 and m is 2-10, 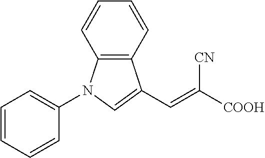 [SMILES: N#C/C(=C\c1cn(-c2ccccc2)c2ccccc12)C(=O)O]
(b) UK5099 (PF-1005023)
or (c) CB839 (Telagenastat) 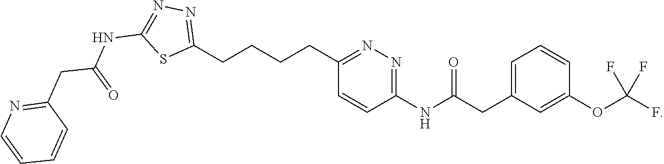 [SMILES: O=C(Cc1cccc(OC(F)(F)F)c1)Nc1ccc(CCCCc2nnc(NC(=O)Cc3ccccn3)s2)nn1]
